(19) United States
(12) Patent Application Publication
(10) Pub. No.: US 2025/0313530 A1
(43) Pub. Date: Oct. 9, 2025

(54) SMALL MOLECULE INHIBITORS OF INTERLEUKIN-4 AND USES THEREOF

(71) Applicant: Trustees of Boston University, Boston, MA (US)

(72) Inventors: Arturo Jose Vegas, Wellesley, MA (US); Raavi, Boston, MA (US); Sean P. Quinnell, San Diego, CA (US); Daniel F. Sheehy, Green Cove Springs, FL (US); Chandler Ruping, Burlington, MA (US)

(73) Assignee: Trustees of Boston University, Boston, MA (US)

(21) Appl. No.: 19/171,111

(22) Filed: Apr. 4, 2025

Related U.S. Application Data
(60) Provisional application No. 63/575,333, filed on Apr. 5, 2024.

(51) Int. Cl. C07D 213/85; A61K 31/44

(52) U.S. Cl. CPC C07D 213/85; A61K 31/44

(57) ABSTRACT

Dysregulated IL-4 signaling contributes to a number of immune-mediated diseases such as allergic inflammation, cancer, and autoimmunity. The present disclosure provides small molecule binders and inhibitors of IL-4, and pharmaceutical compositions, kits, and uses thereof.

### RELATED APPLICATIONS

This application claims priority under 35 U.S.C. 119(e) to U.S. Provisional Application 63/575,333 filed Apr. 5, 2024, the entirety of which is incorporated herein by reference.

### BACKGROUND

Interleukin-4 (IL-4) is an immunoregulator of inflammation and plays a role in the progression of autoimmunity, allergic reactions, arthritis, and cancer¹. In asthma, IL-4 induces airway inflammation, obstruction, and hyperresponsiveness^{2,3}, while in cancer IL-4 activity is linked to promoting tumor progression, immunosuppression, and increasing tumor resistance to apoptosis. IL-4 engages with two types of cell membrane receptors type I (IL-4Rα & γ_{c}) and type II (IL-4Rα and the IL-13Rα1) for downstream JAK1/2/3-TyK2 mediated phosphorylation induced by ligand-receptor dimerization.⁴⁻⁶ The expression of these IL-4 receptors is determined primarily by tissue, with type I receptor complexes being restricted mainly to hematopoietic cells whereas type 11 expression is more widespread. The dysregulation of cellular signaling mediated by these receptors is affiliated with numerous inflammatory conditions.^{7,8} Further therapeutics targeting IL-4 are needed.

### SUMMARY

In one aspect, provided herein, compound of formula (I):

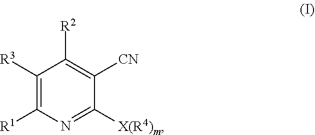

or a pharmaceutically acceptable salt, solvate, hydrate, polymorph, co-crystal, tautomer, stereoisomer, isotopically labeled compound, or prodrug thereof, wherein:
X is N, S, O, or halo;
each of R¹ and R² is independently optionally substituted alkyl, optionally substituted aryl, optionally substituted heteroaryl, optionally substituted heterocyclyl, optionally substituted amino, optionally substituted alkoxy, hydroxyl, or optionally substituted carbocyclyl, wherein at least one of R¹ and R² is optionally substituted cyclopropyl, optionally substituted cyclobutyl, or optionally substituted bicyclopentanyl;
R³ is hydrogen or optionally substituted alkyl,
each R⁴ is independently hydrogen, optionally substituted alkyl, a nitrogen protecting group when appended to a nitrogen, an oxygen protecting group when appended to an oxygen, a sulfur protecting group when appended to a sulfur, or, where X is N, two instances of R⁴, together with the nitrogen atom to which they are attached, form an optionally substituted heterocyclyl or heteroaryl; and
m is 0, 1, or 2.

In another aspect, the present disclosure provides a pharmaceutical composition comprising a compound of the present disclosure, or a pharmaceutically acceptable salt, solvate, hydrate, polymorph, co-crystal, tautomer, stereoisomer, isotopically labeled compound, or prodrug thereof, and a pharmaceutically acceptable carrier.

In one aspect of the present disclosure, provided is a therapeutic composition comprising the compound described herein or the pharmaceutical composition described herein, and an at least one second therapeutic molecule.

In another aspect, provided herein is use of the compound described herein, or a pharmaceutically acceptable salt, solvate, hydrate, polymorph, co-crystal, tautomer, stereoisomer, isotopically labeled compound, or prodrug thereof, or the pharmaceutical composition described herein, or the therapeutic composition described herein, in the manufacture of a medicament for treating a disease.

In one aspect of the present disclosure, provided is a method of treating a disease in a subject in need thereof, the method comprising administering to the subject in need thereof a therapeutically effective amount of a compound described herein, or a pharmaceutically acceptable salt, solvate, hydrate, polymorph, co-crystal, tautomer, stereoisomer, isotopically labeled derivative, or prodrug thereof, or the pharmaceutical composition described herein, or the therapeutic composition described herein.

In another aspect, provided herein is a method of reducing an inflammatory response in a subject in need thereof, the method comprises administering to the subject a composition described herein or a pharmaceutically acceptable salt, solvate, hydrate, polymorph, co-crystal, tautomer, stereoisomer, isotopically labeled compound, or prodrug thereof, or the pharmaceutical composition described herein, or the therapeutic composition described herein.

In one aspect, described herein is a method of inhibiting the activity of a cytokine in a subject in need thereof, the method comprising administering to the subject an effective amount of a compound described herein or a pharmaceutically acceptable salt, solvate, hydrate, polymorph, co-crystal, tautomer, stereoisomer, isotopically labeled compound, or prodrug thereof, or the pharmaceutical composition described herein or the therapeutic composition described herein.

In another aspect, provided herein is a method of inhibiting the activity of IL-4 in a biological sample or cell, the method comprising administering to the biological sample or cell an effective amount of a compound described herein or a pharmaceutically acceptable salt, solvate, hydrate, polymorph, co-crystal, tautomer, stereoisomer, isotopically labeled compound, or prodrug thereof, or the pharmaceutical composition described herein, or the therapeutic composition described herein.

In one aspect of the present disclosure, provided is a kit comprising: a compound described herein or a pharmaceutically acceptable salt, solvate, hydrate, polymorph, co-crystal, tautomer, stereoisomer, isotopically labeled compound, or prodrug thereof, or the pharmaceutical composition described herein, or the therapeutic composition described herein, and instructions for using the compound, or a pharmaceutically acceptable salt, solvate, hydrate, polymorph, co-crystal, tautomer, stereoisomer, isotopically labeled compound, or prodrug thereof, or the pharmaceutical composition or the therapeutic composition.

The details of certain embodiments of the invention are set forth in the Detailed Description of Certain Embodiments, as described below. Other features, objects, and advantages of the invention will be apparent from the Definitions, Examples, and Claims.

### BRIEF DESCRIPTION OF THE DRAWINGS

FIGS. 1A-1C shows Nico-52 Analogs and synthesis scheme for investigating structure-activity relationships. FIG. 1A shows structural changes at the three rings comprising Nico-52 to establish which features contribute to Nico-52's binding to IL-4. FIG. 1B shows the synthesis of Nico-52 and amino nicotinonitrile containing R1 and R3 analogs. FIG. 1C Shows the synthesis of hydroxy nicotinonitrile Nico-52 analogs.

FIGS. 2A-2D show Nico-52 analogs dose response in HEK Blue IL-4/IL-13 reporter assay and selectivity across structurally and functionally related cytokines. FIG. 2A shows dose dependence and EC₅₀ of 14 and 15 in the HEK Blue IL-4/IL-13 reporter assay. FIG. 2B shows IL-13 inhibition by analogs 14 and 15 in the HEK Blue IL-4/IL-13 reporter assay.

FIG. 2C shows thermal shift analysis of Nico-52, 14 and 15 against IL-2, IL-4, IL-7, IL-13, and IL-21 at 2 μM (0.1% DMSO) final concentration, a cut-off=0.5° C. was used to determine the threshold for the determination of binding events. FIG. 2D shows hill plots of Nico-52, 14 and 15 dose responses in the HEK Blue IL-4/IL-13 reporter assay. The Hill coefficients were determined for Nico-52=1.24±0.08, 14=1.69±0.25, and for analog 15=1.49±0.15.

FIGS. 3A-3O shows dose-dependent inhibition of IL-4 induced STAT-6 phosphorylation in THP-1 monocytes with analogs 14 and 15 (type 2 IL-4 receptor). FIG. 3A shows western blots of pSTAT-6 to STAT-6 upon treatment with IL-4 and vehicle or analog 14. FIG. 3B shows dose-dependent IL-4 inhibition with 14 in THP-1 monocytes, normalized to STAT-6 levels. FIGS. 3C-3G shows immunofluorescence of THP-1 monocytes upon treatment with (C) vehicle alone, (D) IL-4+ vehicle, (E) IL-4+50 nM 14, (F) IL-4+500 nM 14, (G) IL-4+5 μM 14. FIG. 3H shows western blots of pSTAT-6 to STAT-6 upon treatment with IL-4 and vehicle or analog 15. FIG. 3I shows dose-dependent IL-4 inhibition with 15 in THP-1 monocytes, normalized to STAT-6 levels. FIGS. 3J-3O shows immunofluorescence of THP-1 monocytes upon treatment with (J) vehicle alone, (K) IL-4+ vehicle (L) IL-4+5 nM 15, (M) IL-4+50 nM 15, (N) IL-4+500 nM 15, (0) IL-4+5 μM 15 (Dark blue: pSTAT-6, Cyan: nucleus).

FIGS. 4A-4B shows Nico-52 and analogs dose-dependent inhibition of IL-4 induced STAT6 phosphorylation in Ramos B lymphocytes (type I IL-4 receptor). FIG. 4A shows western blots of pSTAT-6 and STAT-6 upon treatment with IL-4 and vehicle/Nico-52. Nico-52 quantified dose-dependent IL-4 inhibition in Ramos B lymphocytes normalized to STAT-6. FIG. 4B shows western blots of pSTAT-6 to STAT-6 upon treatment with IL-4 with vehicle/Nico-52/14/15. Quantified Nico-52, 14 and 15 percent IL-4 inhibition at 50 nM, 500 nM, and 5 μM, normalized to STAT-6. *=p<0.05, **=p<0.01, ****=p<0.0001, ns=not significant.

FIGS. 5A-5B shows binding affinity determination of analog 15 with SPR. FIG. 5A shows Kinetic Sensorgrams of analog 15 binding from the low to high concentrations listed. FIG. 5B shows steady-state affinity binding analysis of analog 15 to IL-4, with a measured K_{D} of 31.9 nM.

FIG. 6 shows a summary schematic depicting Nico-52 structure-activity relationship determination to identify active analogs.

FIGS. 7A-7C show HPLC chromatograms of representative analogs: Compound 1 (FIG. 7A, top), Compound 2 (FIG. 7A, bottom), Compound 10 (FIG. 7B, top), Compound 11 (FIG. 7B, bottom). Compound 12 (FIG. 7C, top), and Compound 33 (FIG. 7C, bottom).

FIGS. 8A-8B show LCMS chromatograms of representative analogs: Compound 6 (FIG. 8A, left), Compound 8 (FIG. 8A, right), Compound 9 (FIG. 8B, left), and Compound 32 (FIG. 8B, right).

FIG. 9 shows pSTAT-6 Western blot for THP-1 cells for analog 14.

FIG. 10 shows STAT-6 Western blot for THP-1 cells for analog 14.

FIG. 11 shows pSTAT-6 Western blot for THP-1 cells for analog 15.

FIG. 12 shows STAT-6 Western blot for THP-1 cells for analog 15.

FIG. 13 shows pSTAT-6 Western blot for Ramos cells for Nico-52.

FIG. 14 shows STAT-6 Western blot for Ramos cells for Nico-52.

FIG. 15 shows pSTAT-6 Western blot for Ramos cells for analogs 14 and 15.

FIG. 16 shows STAT-6 Western blot for Ramos cells for analogs 14 and 15.

FIG. 17 shows pSTAT-6 Western blot for Ramos cells for Nico-52.

FIG. 18 shows STAT-6 Western blot for Ramos cells for Nico-52.

FIG. 19 shows pSTAT-6 Western blot for Raw macrophage cells for Nico-52.

FIG. 20 shows STAT-6 Western blot for Raw macrophage cells for Nico-52.

### DEFINITIONS

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry. University Science Books. Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, in some embodiments, the compounds described herein are in the form of an individual enantiomer, diastereomer or geometric isomer, or are in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN 1972). The invention additionally encompasses compounds as individual isomers substantially free of other isomers, and alternatively, as mixtures of various isomers.

Unless otherwise provided, formulae and structures depicted herein include compounds that do not include isotopically enriched atoms, and also include compounds that include isotopically enriched atoms. For example, compounds having the present structures except for the replacement of hydrogen by deuterium or tritium, replacement of ¹⁹F with ¹⁸F, or the replacement of a carbon by a ¹³C- or ¹⁴C-enriched carbon are within the scope of the disclosure. Such compounds are useful, for example, as analytical tools or probes in biological assays.

The term “isotopes” refers to variants of a particular chemical element such that, while all isotopes of a given element share the same number of protons in each atom of the element, those isotopes differ in the number of neutrons. The term “radioactivity” or “radioactive decay” refers to the process by which a nucleus of an unstable isotope (e.g., ¹⁸F) loses energy by emitting particles or rays (e.g., alpha particles, beta particles, and gamma rays) of ionizing radiation. Such an unstable isotope or a material including the unstable isotope is referred to as “radioactive.” The Curie (C₁) is a non-SI (non-International System of Units) unit of radioactivity and is defined as 1 Ci=3.7×10¹⁰ decays per second. The term “specific activity” refers to the unit radioactivity of a material (e.g., a compound of disclosed herein, or a salt, tautomer, stereoisomer, or isotopically labeled derivative (e.g., ¹⁸F labeled derivative) thereof). In certain embodiments, the term “specific activity” refers to the radioactivity of a material per micromole (μmol) of the material.

The term “heteroatom” refers to an atom that is not hydrogen or carbon. In certain embodiments, the heteroatom is nitrogen. In certain embodiments, the heteroatom is oxygen. In certain embodiments, the heteroatom is sulfur.

When a range of values (“range”) is listed, it encompasses each value and sub-range within the range. A range is inclusive of the values at the two ends of the range unless otherwise provided. For example “C₁₋₆ alkyl” encompasses, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some embodiments, an alkyl group has 1 to 12 carbon atoms (“C₁₋₁₂ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain embodiments, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain embodiments, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl and refers to an alkyl group wherein all of the hydrogen atoms are independently replaced by a halogen, e.g., fluoro, bromo, chloro, or iodo. In some embodiments, the haloalkyl moiety has 1 to 20 carbon atoms (“C₁₋₂₀ haloalkyl”). In some embodiments, the haloalkyl moiety has 1 to 10 carbon atoms (“C₁₋₁₀ haloalkyl”). In some embodiments, the haloalkyl moiety has 1 to 9 carbon atoms (“C₁₋₉ haloalkyl”). In some embodiments, the haloalkyl moiety has 1 to 8 carbon atoms (“C₁₋₈ haloalkyl”). In some embodiments, the haloalkyl moiety has 1 to 7 carbon atoms (“C₁₋₇ haloalkyl”). In some embodiments, the haloalkyl moiety has 1 to 6 carbon atoms (“C₁₋₆ haloalkyl”). In some embodiments, the haloalkyl moiety has 1 to 5 carbon atoms (“C₁₋₅ haloalkyl”). In some embodiments, the haloalkyl moiety has 1 to 4 carbon atoms (“C₁₋₄ haloalkyl”). In some embodiments, the haloalkyl moiety has 1 to 3 carbon atoms (“C₁₋₃ haloalkyl”). In some embodiments, the haloalkyl moiety has 1 to 2 carbon atoms (“C₁₋₂ haloalkyl”). In some embodiments, all haloalkyl hydrogen atoms are independently replaced with fluoro to provide a “perfluoroalkyl” group. In some embodiments, all haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂Cl, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkyl group refers to a saturated group having from 1 to 20 carbon atoms and 1 or more heteroatoms within the parent chain (“heteroC₁₋₂₀ alkyl”). In certain embodiments, a heteroalkyl group refers to a saturated group having from 1 to 12 carbon atoms and 1 or more heteroatoms within the parent chain (“heteroC₁₋₁₂ alkyl”). In some embodiments, a heteroalkyl group is a saturated group having 1 to 11 carbon atoms and 1 or more heteroatoms within the parent chain (“heteroC₁₋₁₁ alkyl”). In some embodiments, a heteroalkyl group is a saturated group having 1 to 10 carbon atoms and 1 or more heteroatoms within the parent chain (“heteroC₁₋₁₀ alkyl”). In some embodiments, a heteroalkyl group is a saturated group having 1 to 9 carbon atoms and 1 or more heteroatoms within the parent chain (“heteroC₁₋₉ alkyl”). In some embodiments, a heteroalkyl group is a saturated group having 1 to 8 carbon atoms and 1 or more heteroatoms within the parent chain (“heteroC₁₋₈ alkyl”). In some embodiments, a heteroalkyl group is a saturated group having 1 to 7 carbon atoms and 1 or more heteroatoms within the parent chain (“heteroC₁₋₇ alkyl”). In some embodiments, a heteroalkyl group is a saturated group having 1 to 6 carbon atoms and 1 or more heteroatoms within the parent chain (“heteroC₁₋₆ alkyl”). In some embodiments, a heteroalkyl group is a saturated group having 1 to 5 carbon atoms and 1 or 2 heteroatoms within the parent chain (“heteroC₁₋₅ alkyl”). In some embodiments, a heteroalkyl group is a saturated group having 1 to 4 carbon atoms and 1 or 2 heteroatoms within the parent chain (“heteroC₁₋₄ alkyl”). In some embodiments, a heteroalkyl group is a saturated group having 1 to 3 carbon atoms and 1 heteroatom within the parent chain (“heteroC₁₋₃ alkyl”). In some embodiments, a heteroalkyl group is a saturated group having 1 to 2 carbon atoms and 1 heteroatom within the parent chain (“heteroC₁₋₂ alkyl”). In some embodiments, a heteroalkyl group is a saturated group having 1 carbon atom and 1 heteroatom (“heteroC₁ alkyl”). In some embodiments, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain embodiments, the heteroalkyl group is an unsubstituted heteroC₁₋₁₂ alkyl. In certain embodiments, the heteroalkyl group is a substituted heteroC₁₋₁₂ alkyl.

The term “alkenyl” refers to a radical of a straight-chain or branched hydrocarbon group having from 1 to 20 carbon atoms and one or more carbon-carbon double bonds (e.g., 1, 2, 3, or 4 double bonds). In some embodiments, an alkenyl group has 1 to 20 carbon atoms (“C₁₋₂₀ alkenyl”). In some embodiments, an alkenyl group has 1 to 12 carbon atoms (“C₁₋₁₂ alkenyl”). In some embodiments, an alkenyl group has 1 to 11 carbon atoms (“C₁₋₁₁ alkenyl”). In some embodiments, an alkenyl group has 1 to 10 carbon atoms (“C₁₋₁₀ alkenyl”). In some embodiments, an alkenyl group has 1 to 9 carbon atoms (“C₁₋₉ alkenyl”). In some embodiments, an alkenyl group has 1 to 8 carbon atoms (“C₁₋₈ alkenyl”). In some embodiments, an alkenyl group has 1 to 7 carbon atoms (“C₁₋₇ alkenyl”). In some embodiments, an alkenyl group has 1 to 6 carbon atoms (“C₁₋₆ alkenyl”). In some embodiments, an alkenyl group has 1 to 5 carbon atoms (“C₁₋₅ alkenyl”). In some embodiments, an alkenyl group has 1 to 4 carbon atoms (“C₁₋₄ alkenyl”). In some embodiments, an alkenyl group has 1 to 3 carbon atoms (“C₁₋₃ alkenyl”). In some embodiments, an alkenyl group has 1 to 2 carbon atoms (“C₁₋₂ alkenyl”). In some embodiments, an alkenyl group has 1 carbon atom (“C₁ alkenyl”). In some embodiments, the one or more carbon-carbon double bonds are internal (such as in 2-butenyl) or terminal (such as in 1-butenyl). Examples of C₁₋₄ alkenyl groups include methylindenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₁₋₂₀ alkenyl. In certain embodiments, the alkenyl group is a substituted C₁₋₂₀ alkenyl. In an alkenyl group, a C═C double bond for which the stereochemistry is not specified (e.g., —CH═CHCH₃ or

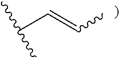

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkenyl group refers to a group having from 1 to 20 carbon atoms, at least one double bond, and 1 or more heteroatoms within the parent chain (“heteroC₁₋₂₀ alkenyl”). In certain embodiments, a heteroalkenyl group refers to a group having from 1 to 12 carbon atoms, at least one double bond, and 1 or more heteroatoms within the parent chain (“heteroC₁₋₁₂ alkenyl”). In certain embodiments, a heteroalkenyl group refers to a group having from 1 to 11 carbon atoms, at least one double bond, and 1 or more heteroatoms within the parent chain (“heteroC₁₋₁₁ alkenyl”). In certain embodiments, a heteroalkenyl group refers to a group having from 1 to 10 carbon atoms, at least one double bond, and 1 or more heteroatoms within the parent chain (“heteroC₁₋₁₀ alkenyl”). In some embodiments, a heteroalkenyl group has 1 to 9 carbon atoms at least one double bond, and 1 or more heteroatoms within the parent chain (“heteroC₁₋₉ alkenyl”). In some embodiments, a heteroalkenyl group has 1 to 8 carbon atoms, at least one double bond, and 1 or more heteroatoms within the parent chain (“heteroC₁₋₈ alkenyl”). In some embodiments, a heteroalkenyl group has 1 to 7 carbon atoms, at least one double bond, and 1 or more heteroatoms within the parent chain (“heteroC₁₋₇ alkenyl”). In some embodiments, a heteroalkenyl group has 1 to 6 carbon atoms, at least one double bond, and 1 or more heteroatoms within the parent chain (“heteroC₁₋₆ alkenyl”). In some embodiments, a heteroalkenyl group has 1 to 5 carbon atoms, at least one double bond, and 1 or 2 heteroatoms within the parent chain (“heteroC₁₋₅ alkenyl”). In some embodiments, a heteroalkenyl group has 1 to 4 carbon atoms, at least one double bond, and 1 or 2 heteroatoms within the parent chain (“heteroC₁₋₄ alkenyl”). In some embodiments, a heteroalkenyl group has 1 to 3 carbon atoms, at least one double bond, and 1 heteroatom within the parent chain (“heteroC₁₋₃ alkenyl”). In some embodiments, a heteroalkenyl group has 1 to 2 carbon atoms, at least one double bond, and 1 heteroatom within the parent chain (“heteroC₁₋₂ alkenyl”). In some embodiments, a heteroalkenyl group has 1 to 6 carbon atoms, at least one double bond, and 1 or 2 heteroatoms within the parent chain (“heteroC₁₋₆ alkenyl”). Unless otherwise specified, each instance of a heteroalkenyl group is independently unsubstituted (an “unsubstituted heteroalkenyl”) or substituted (a “substituted heteroalkenyl”) with one or more substituents. In certain embodiments, the heteroalkenyl group is an unsubstituted heteroC₁₋₂₀ alkenyl. In certain embodiments, the heteroalkenyl group is a substituted heteroC₁₋₂₀ alkenyl.

The term “alkynyl” refers to a radical of a straight-chain or branched hydrocarbon group having from 1 to 20 carbon atoms and one or more carbon-carbon triple bonds (e.g., 1, 2, 3, or 4 triple bonds) (“C₁₋₂₀ alkynyl”). In some embodiments, an alkynyl group has 1 to 10 carbon atoms (“C₁₋₁₀ alkynyl”). In some embodiments, an alkynyl group has 1 to 9 carbon atoms (“C₁₋₉ alkynyl”). In some embodiments, an alkynyl group has 1 to 8 carbon atoms (“C₁₋₈ alkynyl”). In some embodiments, an alkynyl group has 1 to 7 carbon atoms (“C₁₋₇ alkynyl”). In some embodiments, an alkynyl group has 1 to 6 carbon atoms (“C₁₋₆ alkynyl”). In some embodiments, an alkynyl group has 1 to 5 carbon atoms (“C₁₋₅ alkynyl”). In some embodiments, an alkynyl group has 1 to 4 carbon atoms (“C₁₋₄ alkynyl”). In some embodiments, an alkynyl group has 1 to 3 carbon atoms (“C₁₋₃ alkynyl”). In some embodiments, an alkynyl group has 1 to 2 carbon atoms (“C₁₋₂ alkynyl”). In some embodiments, an alkynyl group has 1 carbon atom (“C₁ alkynyl”). In some embodiments, the one or more carbon-carbon triple bonds are internal (such as in 2-butynyl) or terminal (such as in 1-butynyl). Examples of C₁₋₄ alkynyl groups include, without limitation, methylindenyl (C₁), ethynyl (C₂), 1-propynyl (C₃), 2-propynyl (C₃), 1-butynyl (C₄), 2-butynyl (C₄), and the like. Examples of C₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₁₋₂₀ alkynyl. In certain embodiments, the alkynyl group is a substituted C₁₋₂₀ alkynyl.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain embodiments, a heteroalkynyl group refers to a group having from 1 to 20 carbon atoms, at least one triple bond, and 1 or more heteroatoms within the parent chain (“heteroC₁₋₂₀ alkynyl”). In certain embodiments, a heteroalkynyl group refers to a group having from 1 to 10 carbon atoms, at least one triple bond, and 1 or more heteroatoms within the parent chain (“heteroC₁₋₁₀ alkynyl”). In some embodiments, a heteroalkynyl group has 1 to 9 carbon atoms, at least one triple bond, and 1 or more heteroatoms within the parent chain (“heteroC₁₋₉ alkynyl”). In some embodiments, a heteroalkynyl group has 1 to 8 carbon atoms, at least one triple bond, and 1 or more heteroatoms within the parent chain (“heteroC₁₋₈ alkynyl”). In some embodiments, a heteroalkynyl group has 1 to 7 carbon atoms, at least one triple bond, and 1 or more heteroatoms within the parent chain (“heteroC₁₋₇ alkynyl”). In some embodiments, a heteroalkynyl group has 1 to 6 carbon atoms, at least one triple bond, and 1 or more heteroatoms within the parent chain (“heteroC₁₋₆ alkynyl”). In some embodiments, a heteroalkynyl group has 1 to 5 carbon atoms, at least one triple bond, and 1 or 2 heteroatoms within the parent chain (“heteroC₁₋₅ alkynyl”). In some embodiments, a heteroalkynyl group has 1 to 4 carbon atoms, at least one triple bond, and 1 or 2 heteroatoms within the parent chain (“heteroC₁₋₄ alkynyl”). In some embodiments, a heteroalkynyl group has 1 to 3 carbon atoms, at least one triple bond, and 1 heteroatom within the parent chain (“heteroC₁₋₃ alkynyl”). In some embodiments, a heteroalkynyl group has 1 to 2 carbon atoms, at least one triple bond, and 1 heteroatom within the parent chain (“heteroC₁₋₂ alkynyl”). In some embodiments, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents. In certain embodiments, the heteroalkynyl group is an unsubstituted heteroC₁₋₂ alkynyl. In certain embodiments, the heteroalkynyl group is a substituted heteroC₁₋₂₀ alkynyl.

The term “carbocyclyl” or “carbocyclic” refers to a radical of a non-aromatic cyclic hydrocarbon group having from 3 to 14 ring carbon atoms (“C₃₋₁₄ carbocyclyl”) and zero heteroatoms in the non-aromatic ring system. In some embodiments, a carbocyclyl group has 3 to 14 ring carbon atoms (“C₃₋₁₄ carbocyclyl”). In some embodiments, a carbocyclyl group has 3 to 13 ring carbon atoms (“C₃₋₁₃ carbocyclyl”). In some embodiments, a carbocyclyl group has 3 to 12 ring carbon atoms (“C₃₋₁₂ carbocyclyl”). In some embodiments, a carbocyclyl group has 3 to 11 ring carbon atoms (“C₃₋₁₁ carbocyclyl”). In some embodiments, a carbocyclyl group has 3 to 10 ring carbon atoms (“C₃₋₁₀ carbocyclyl”). In some embodiments, a carbocyclyl group has 3 to 8 ring carbon atoms (“C₃₋₈ carbocyclyl”). In some embodiments, a carbocyclyl group has 3 to 7 ring carbon atoms (“C₃₋₇ carbocyclyl”). In some embodiments, a carbocyclyl group has 3 to 6 ring carbon atoms (“C₃₋₆ carbocyclyl”). In some embodiments, a carbocyclyl group has 4 to 6 ring carbon atoms (“C₄₋₆ carbocyclyl”). In some embodiments, a carbocyclyl group has 5 to 6 ring carbon atoms (“C₅₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups as well as cycloundecyl (C₁₁), spiro[5.5]undecanyl (C₁₁), cyclododecyl (C₁₂), cyclododecenyl (C₁₂), cyclotridecane (C₁₃), cyclotetradecane (C₁₄), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and, in some embodiments, are saturated or contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₄ carbocyclyl. In certain embodiments, the carbocyclyl group is a substituted C₃₋₁₄ carbocyclyl.

In some embodiments, “carbocyclyl” is a monocyclic, saturated carbocyclyl group having from 3 to 14 ring carbon atoms (“C₃₋₁₄ cycloalkyl”). In some embodiments, a cycloalkyl group has 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 4 to 6 ring carbon atoms (“C₄₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₄ cycloalkyl. In certain embodiments, the cycloalkyl group is a substituted C₃₋₁₄ cycloalkyl. In certain embodiments, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment is a carbon or nitrogen atom, as valency permits. In some embodiments, a heterocyclyl group is monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and, in some embodiments, is saturated or contains one or more carbon-carbon double or triple bonds. In some embodiments, heterocyclyl polycyclic ring systems include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl. In certain embodiments, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 □ electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 π-electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, in some embodiments the point of attachment is a carbon or nitrogen atom, as valency permits. In some embodiments, heteroaryl polycyclic ring systems include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment is on either ring, e.g., either the ring bearing a heteroatom (e.g., 2-indolyl) or the ring that does not contain a heteroatom (e.g., 5-indolyl). In certain embodiments, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain embodiments, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur. “Heterobiaryl” refers to an instance of two or more aryl rings being fused together, wherein at least one of the aryl rings is heteroaryl.

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepanyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain embodiments, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which is substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl. “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds and includes any of the substituents described herein that results in the formation of a stable compound. The present disclosure contemplates any and all such combinations in order to arrive at a stable compound. For purposes of this disclosure, heteroatoms such as nitrogen may have hydrogen substituents and/or any suitable substituent as described herein which satisfy the valencies of the heteroatoms and results in the formation of a stable moiety. The disclosure is not limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}p(═O)(OR^{cc})₂, —NR^{bb}P(═O)(N(R^{bb})₂), —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl. C₁₋₂₀ alkenyl, C₁₋₂₀ alkynyl, heteroC₁₋₂₀ alkyl, heteroC₁₋₂₀ alkenyl, heteroC₁₋₂₀ alkynyl. C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{bb}, ═NR^{bb}, or ═NOR^{cc};
wherein:
   each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀alkynyl, C₃₋₁₀ carbocyclyl, 3-14 membered heterocyclyl. C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups:
   each instance of R^{bb} is, independently, selected from hydrogen. —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂), C₁₋₂₀ alkyl, C₁₋₂₀ perhaloalkyl, C₁₋₂₀ alkenyl, C₁₋₂₀ alknyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{cc} is, independently, selected from hydrogen. C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl. C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;
   each instance of R^{dd} is, independently, selected from halogen, —CN, —NO₂, —N₃, —SO₂H, —SO₃H, —OH, —OR^{cc}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R′)₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents are joined to form ═O or ═S;
   each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{BB} groups;
   each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl, and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;
   each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl) ⁺X⁻, —NH₃⁺X⁻, —N(OC₁₋₆ alkyl)C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₄ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆(alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R^{gg} substituents are joined to form ═O or ═S; and
   each X⁻ is a counterion.

In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain embodiments, each carbon atom substituent is independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb}), —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) w % ben attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain embodiments, each carbon atom substituent is independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain embodiments, each carbon atom substituent is independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom, and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain embodiments, the molecular weight of a carbon atom substituent is lower than 250, lower than 200, lower than 150, lower than 100, or lower than 50 g/mol. In certain embodiments, a carbon atom substituent consists of carbon, hydrogen, fluorine, chlorine, bromine, iodine, oxygen, sulfur, nitrogen, and/or silicon atoms. In certain embodiments, a carbon atom substituent consists of carbon, hydrogen, fluorine, chlorine, bromine, iodine, oxygen, sulfur, and/or nitrogen atoms. In certain embodiments, a carbon atom substituent consists of carbon, hydrogen, fluorine, chlorine, bromine, and/or iodine atoms. In certain embodiments, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxy,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{bb})₃, —OP(R^{cc})₂, —OP(R^{cc})₃⁺X⁻, —OP(OR^{cc})₂, —OP(OR^{cc})₃⁺X⁻, —OP(═O)(R^{aa})₂, —OP(═O)(OR^{cc})₂, and —OP(═O)(N(R^{bb}))₂, wherein X⁻, R^{aa}, R^{bb}, and R^{cc} are as defined herein.

The term “alcohol” us herein, refers to an optionally substituted alkyl group, as defined herein, appended to a hydroxyl group. Representative examples of alcohol groups include but are not limited to, methanol, ethanol, n-propanol, isopropanol, n-butanol, isobutanol, and t-butanol.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy and tert-butoxy.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain embodiments, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂ wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

In some embodiments, nitrogen atoms are substituted or unsubstituted as valency permits, and include primary, secondary, tertiary, and quaternary nitrogen atoms. Exemplary nitrogen atom substituents include hydrogen, —OH, —OR^{aa}, —N(R^{cc})₂, —CN, —C(═O)R^{aa}, —C(═O)N(R^{cc})₂, —CO₂R^{cc}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, hetero C₁₋₂₀ alkyl, hetero C₁₋₂₀ alkenyl, hetero C₁₋₂₀) alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, each nitrogen atom substituent is independently substituted (e.g., substituted with one or more halogen) or unsubstituted C₁₋₆ alkyl, —C(═O)R^{aa}, —CO₂R^{aa}, —C(═O)N(R^{bb})₂, or a nitrogen protecting group. In certain embodiments, each nitrogen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a nitrogen protecting group, wherein R^{aa} is hydrogen, substituted (e.g., substituted with one or more halogen) or unsubstituted C₁₋₁₀ alkyl, or an oxygen protecting group when attached to an oxygen atom, and each R^{bb} is independently hydrogen, substituted (e.g., substituted with one or more halogen) or unsubstituted C₁₋₁₀ alkyl, or a nitrogen protecting group. In certain embodiments, each nitrogen atom substituent is independently substituted (e.g., substituted with one or more halogen) or unsubstituted C₁₋₆ alkyl or a nitrogen protecting group.

In certain embodiments, the substituent present on the nitrogen atom is a nitrogen protecting group (also referred to herein as an “amino protecting group”). Nitrogen protecting groups include —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc} C₁₋₁₀ alkyl (e.g., aralkyl, heteroaralkyl), C₁₋₂₀ alkenyl, C₁₋₂₀ alknyl, hetero C₁₋₂₀ alkyl, hetero C₁₋₂₀ alkenyl, hetero C₁₋₂₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, heteroalkyl, heteroalkenyl, heteroalkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, in certain embodiments, at least one nitrogen protecting group is an amide group (e.g., a moiety that include the nitrogen atom to which the nitrogen protecting groups (e.g., —C(═O)R^{aa}) is directly attached). In certain such embodiments, each nitrogen protecting group, together with the nitrogen atom to which the nitrogen protecting group is attached, is independently selected from the group consisting of formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivatives, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivatives, o-nitrobenzamide, and o-(benzoyloxymethyl)benzamide.

In certain embodiments, at least one nitrogen protecting group is a carbamate group (e.g., a moiety that include the nitrogen atom to which the nitrogen protecting groups (e.g., —C(═O)OR^{aa}) is directly attached). In certain such embodiments, each nitrogen protecting group, together with the nitrogen atom to which the nitrogen protecting group is attached, is independently selected from the group consisting of methyl carbamate, ethyl carbamate, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, 1-butyl carbamate (BOC or Boc), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitrobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isobornyl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

In certain embodiments, at least one nitrogen protecting group is a sulfonamide group (e.g., a moiety that include the nitrogen atom to which the nitrogen protecting groups (e.g., —S(═O)₂R^{aa}) is directly attached). In certain such embodiments, each nitrogen protecting group, together with the nitrogen atom to which the nitrogen protecting group is attached, is independently selected from the group consisting of p-toluenesulfonamide (Ts), benzenesulfonamide, 2,3,6-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), p-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

In certain embodiments, each nitrogen protecting group, together with the nitrogen atom to which the nitrogen protecting group is attached, is independently selected from the group consisting of phenothiazinyl-(10)-acyl derivatives, N′-p-toluenesulfonylaminoacyl derivatives, N′-phenylaminothioacyl derivatives, N-benzoylphenylalanyl derivatives. N-acetylmethionine derivatives, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivatives, N-diphenylborinic acid derivatives, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys). In some embodiments, two instances of a nitrogen protecting group together with the nitrogen atoms to which the nitrogen protecting groups are attached are N,N′-isopropylidenediamine.

In certain embodiments, at least one nitrogen protecting group is Bn, Boc, Cbz, Fmoc, trifluoroacetyl, triphenylmethyl, acetyl, or Ts.

In certain embodiments, each oxygen atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or an oxygen protecting group. In certain embodiments, each oxygen atom substituents is independently substituted (e.g., substituted with one or more halogen) or unsubstituted C₁₋₆ alkyl, —C(═O)R^{aa}, —CO₂R^{aa}, —C(═O)N(R^{bb})₂, or an oxygen protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each oxygen atom substituent is independently substituted (e.g., substituted with one or more halogen) or unsubstituted C₁₋₆ alkyl or an oxygen protecting group.

In certain embodiments, the substituent present on an oxygen atom is an oxygen protecting group (also referred to herein as an “hydroxyl protecting group”). Oxygen protecting groups include —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, each oxygen protecting group, together with the oxygen atom to which the oxygen protecting group is attached, is selected from the group consisting of methyl, methoxymethyl (MOM), methylthiomethyl (MTM), t-butylthiomethyl. (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7α-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl (PMB), 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, 4,4′-dimethoxytrityl (4,4′-dimethoxytriphenylmethyl or DMT), α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 4,4′-Dimethoxy-3′″[N-(imidazolylmethyl)]trityl Ether (IDTr-OR), 4,4′-Dimethoxy-3′″-[N-(imidazolylethyl)carbamoyl]trityl Ether (IETr-OR), 1,1-bis(4-methoxyphenyl)-1′-pyrenylmethyl, 9-anthryl, 9-(9-phenyl)xanthenyl, 9-(9-phenyl-10-oxo)anthryl, 1,3-benzodithiolan-2-yl, benzisothiazolyl S,S-dioxido, trimethylsilyl (TMS), triethylsilyl (TES), triisopropylsilyl (TIPS), dimethylisopropylsilyl (TPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), methyl carbonate, 9-fluorenylmethyl carbonate (Fmoc), ethyl carbonate, 2,2,2-trichloroethyl carbonate (Troc), 2-(trimethylsilyl)ethyl carbonate (TMSEC), 2-(phenylsulfonyl)ethyl carbonate (Psec), 2-(triphenylphosphonio)ethyl carbonate (Peoc), isobutyl carbonate, vinyl carbonate, allyl carbonate, 1-butyl carbonate (BOC or Boc), p-nitrophenyl carbonate, benzyl carbonate, p-methoxybenzyl carbonate, 3,4-dimethoxybenzyl carbonate, o-nitrobenzyl carbonate, p-nitrobenzyl carbonate, S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl carbonate (MTMEC-OR), 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, at least one oxygen protecting group is silyl, TBDPS, TBDMS, TIPS, TES, TMS, MOM, THP, t-Bu, Bn, allyl, acetyl, pivaloyl, or benzoyl.

In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group. In certain embodiments, each sulfur atom substituent is independently substituted (e.g., substituted with one or more halogen) or unsubstituted C₁₋₁₀ alkyl, —C(═O)R^{aa}, —CO₂R^{aa}, —C(═O)N(R^{bb})₂, or a sulfur protecting group, wherein R^{aa} is hydrogen, substituted (e.g., substituted with one or more halogen) or unsubstituted C₁₋₁₀ alkyl, or an oxygen protecting group when attached to an oxygen atom; and each R^{bb} is independently hydrogen, substituted (e.g., substituted with one or more halogen) or unsubstituted C₁₋₁₀ alkyl, or a nitrogen protecting group. In certain embodiments, each sulfur atom substituent is independently substituted (e.g., substituted with one or more halogen) or unsubstituted C₁₋₆ alkyl or a sulfur protecting group.

In certain embodiments, the substituent present on a sulfur atom is a sulfur protecting group (also referred to as a “thiol protecting group”). In some embodiments, each sulfur protecting group is selected from the group consisting of —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

In certain embodiments, the molecular weight of a substituent is lower than 250, lower than 200, lower than 150, lower than 100, or lower than 50 g/mol. In certain embodiments, a substituent consists of carbon, hydrogen, fluorine, chlorine, bromine, iodine, oxygen, sulfur, nitrogen, and/or silicon atoms. In certain embodiments, a substituent consists of carbon, hydrogen, fluorine, chlorine, bromine, iodine, oxygen, sulfur, and/or nitrogen atoms. In certain embodiments, a substituent consists of carbon, hydrogen, fluorine, chlorine, bromine, and/or iodine atoms. In certain embodiments, a substituent consists of carbon, hydrogen, fluorine, and/or chlorine atoms. In certain embodiments, a substituent comprises 0, 1, 2, or 3 hydrogen bond donors. In certain embodiments, a substituent comprises 0, 1, 2, or 3 hydrogen bond acceptors.

A “counterion” or “anionic counterion” is a negatively charged group associated with a positively charged group in order to maintain electronic neutrality. An anionic counterion may be monovalent (e.g., including one formal negative charge). An anionic counterion may also be multivalent (e.g., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methanesulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]—, B(C₆F₅)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

Use of the phrase “at least one instance” refers to 1, 2, 3, 4, or more instances, but also encompasses a range, e.g., for example, from 1 to 4, from 1 to 3, from 1 to 2, from 2 to 4, from 2 to 3, or from 3 to 4 instances, inclusive.

The disclosure is not intended to be limited in any manner by the above exemplary listing of substituents. These and other exemplary substituents are described in more detail in the Detailed Description, Examples, and Claims.

### Other Definitions

The following definitions are more general terms used throughout the present application.

As used herein, the term “small molecule” refers to a chemical agent which can include, but is not limited to, a peptide, a peptidomimetic, an amino acid, an amino acid analog, a polynucleotide, a polynucleotide analog, an aptamer, a nucleotide, a nucleotide analog, an organic or inorganic compound (i.e., including heterorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

The small molecule may contain multiple carbon-carbon bonds, stereocenters, and other functional groups (e.g., amines, hydroxyl, carbonyls, and heterocyclic rings, etc.). In certain embodiments, the molecular weight of a small molecule is not more than about 1,000 g/mol, not more than about 900 g/mol, not more than about 800 g/mol, not more than about 700 g/mol, not more than about 600 g/mol, not more than about 500 g/mol, not more than about 400 g/mol, not more than about 300 g/mol, not more than about 200 g/mol, or not more than about 100 g/mol. In certain embodiments, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and not more than about 500 g/mol) are also possible. In certain embodiments, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. Small molecules include, but are not limited to, radionuclides and imaging agents. In certain embodiments, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present disclosure.

The term “scaffold” used herein refers to a substance used to provide support and/or carry the molecule of interest. In some embodiments, the scaffold is a lipid bilayer or solid surface. In some embodiments, a lipid bilayer surface is a liposome. In some embodiments, the surface is a solid surface or solid support. In some embodiments, the scaffold is beads (such as magnetic beads, polystyrene beads, or gold beads); resin; fiber; sheet; biocompatible polymer or material, a nanoparticle; a matrix; a hydrogel; a biomaterial, biocompatible, and/or biodegradable scaffold material; or the like. In some embodiments, the scaffold includes a nanocarrier. For example, various nanocarriers for targeting to, e.g., cancer tumors (e.g., via the enhanced permeability and retention effect) are known in the art. In some embodiments, the nanocarrier is PLGA nanoparticles, poly(carboxyphenoxypropane/sebacic acid), poly(glycerol monostearate-co-caprolactone), or the like. Such nanocarriers and their use are described in the art, e.g., Rosenblum et al. Nature Communications 2018 9:1410; which is incorporated by reference herein in its entirety.

Suitable materials for a scaffold surface include, without limitation, a synthetic polymer, biopolymer, latex, or silica. Such materials are well known in the art. For example, the use of beads and/or particles is known in the art and described, e.g. magnetic bead and nano-particles are well known and methods for their preparation have been described in the art for example in U.S. Pat. Nos. 6,878,445; 5,543,158; 5,578,325; 6,676,729; 6,045,925 and 7,462,446, and U.S. Pat. Pub. Nos.: 2005/0025971; 2005/0200438; 2005/0201941; 2005/0271745; 2006/0228551; 2006/0233712; 2007/01666232 and 2007/0264199, contents of all of which are herein incorporated by reference in their entirety.

As used herein, the term “salt” refers to any and all salts and encompasses pharmaceutically acceptable salts. Salts include ionic compounds that result from the neutralization reaction of an acid and a base. A salt is composed of one or more cations (positively charged ions) and one or more anions (negative ions) so that the salt is electrically neutral (without a net charge). Salts of the compounds of the present disclosure include those derived from inorganic and organic acids and bases. Examples of acid addition salts are salts of an amino group formed with inorganic acids, such as hydrochloric acid, hydrobromic acid, phosphoric acid, sulfuric acid, and perchloric acid, or with organic acids, such as acetic acid, oxalic acid, maleic acid, tartaric acid, citric acid, succinic acid, or malonic acid or by using other methods known in the art such as ion exchange. Other salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate, hippurate, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further salts include ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of the present disclosure include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound, or a salt thereof, that are associated with a solvent, usually by a solvolysis reaction. This physical association may include hydrogen bonding. Conventional solvents include water, methanol, ethanol, acetic acid, DMSO, THF, diethyl ether, and the like. The compounds described herein may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolatable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound, and x is a number greater than 0. A given compound may form more than one type of hydrate, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol, amide-to-imide, lactam-to-lactim, enamine-to-imine, and enamine-to-(a different enamine) tautomerizations.

It is also to be understood that compounds that have the same molecular formula but differ in the nature or sequence of bonding of their atoms or the arrangement of their atoms in space are termed “isomers”. Isomers that differ in the arrangement of their atoms in space are termed “stereoisomers”.

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The term “crystalline” or “crystalline form” refers to a solid form substantially exhibiting three-dimensional order. In certain embodiments, a crystalline form of a solid is a solid form that is substantially not amorphous. In certain embodiments, the X-ray powder diffraction (XRPD) pattern of a crystalline form includes one or more sharply defined peaks.

The term “co-crystal” refers to a crystalline structure comprising at least two different components (e.g., a compound disclosed herein and an acid), wherein each of the components is independently an atom, ion, or molecule. In certain embodiments, none of the components is a solvent. In certain embodiments, at least one of the components is a solvent. A co-crystal of a compound disclosed herein and an acid is different from a salt formed from a compound disclosed herein and the acid. In the salt, a compound disclosed herein is complexed with the acid in a way that proton transfer (e.g., a complete proton transfer) from the acid to a compound disclosed herein easily occurs at room temperature. In the co-crystal, however, a compound disclosed herein is complexed with the acid in a way that proton transfer from the acid to a compound disclosed herein does not easily occur at room temperature. In certain embodiments, in the co-crystal, there is no proton transfer from the acid to a compound disclosed herein. In certain embodiments, in the co-crystal, there is partial proton transfer from the acid to a compound disclosed herein. Co-crystals may be useful to improve the properties (e.g., solubility, stability, and ease of formulation) of a compound disclosed herein.

The term “polymorph” refers to a crystalline form of a compound (or a salt, hydrate, or solvate thereof). All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refers to compounds that have cleavable groups and become by solvolysis or under physiological conditions the compounds described herein, which are pharmaceutically active in vivo. Such examples include, but are not limited to, choline ester derivatives and the like. N-alkylmorpholine esters and the like. Other derivatives of the compounds described herein have activity in both their acid and acid derivative forms, but in the acid sensitive form often offer advantages of solubility, tissue compatibility, or delayed release in the mammalian organism (see, Bundgaard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds described herein are particular prodrugs. In some cases it is desirable to prepare double ester type prodrugs such as (acyloxy)alkyl esters or ((alkoxycarbonyl)oxy)alkylesters. C₁-C₈ alkyl, C2-C8 alkenyl, C2-C8 alkynyl, aryl, C7-C12 substituted aryl, and C7-C12 arylalkyl esters of the compounds described herein may be preferred.

The terms “composition” and “formulation” are used interchangeably.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease.

The term “consisting of” refers to compositions, methods, and respective components thereof as described herein, which are exclusive of any element not recited in that description of the embodiment.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments or organelles (such as obtained by lysing cells and separating the components thereof by centrifugation or otherwise). Other examples of biological samples include blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample.

The term “administer,” “administering,” or “administration” refers to implanting, absorbing, ingesting, injecting, inhaling, or otherwise introducing a compound described herein, or a composition thereof, in or on a subject.

The terms “treatment,” “treat.” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

An “effective amount” of a compound described herein refers to an amount sufficient to elicit the desired biological response. An effective amount of a compound described herein may vary depending on such factors as the desired biological endpoint, severity of side effects, disease, or disorder, the identity, pharmacokinetics, and pharmacodynamics of the particular compound, the condition being treated, the mode, route, and desired or required frequency of administration, the species, age and health or general condition of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses. In certain embodiments, the desired dosage is delivered three times a day, two times a day, once a day, every other day, every third day, every week, every two weeks, every three weeks, or every four weeks. In certain embodiments, the desired dosage is delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human comprises about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of the present disclosure are administered orally or parenterally at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. In some embodiment, the therapeutically effective amount encompasses an amount that improves overall therapy, reduces or avoids symptoms, signs, or causes of the condition, and/or enhances the therapeutic efficacy of another therapeutic agent. In certain embodiments, a therapeutically effective amount is an amount sufficient for inhibiting activity of the cytokine. In certain embodiments, a therapeutically effective amount is an amount sufficient for increasing inflammatory response. In certain embodiments, a therapeutically effective amount is an amount sufficient for treating a disease or condition as disclosed herein.

The term “antagonist” or “inhibitor” refers to any agent or entity capable of inhibiting the expression or activity of a target, e.g., IL-4, protein or polypeptide portion thereof. The antagonist may operate via either direct (e.g., by binding to IL-4) or indirect action. In some embodiments the inhibitor binds specifically to IL-4. Methods for measuring the activity of a target, e.g., IL-4, are known in the art and include the IL-4 binding assays (e.g., SMM and DSF thermal shift assays) or the HEK-Blue IL-4 cell-based reporting assay described in the Examples.

In some embodiments, the term refers to a reduction of the level of activity, e.g., IL-4 activity, to a level that is less than 75%, less than 50′%, less than 40%, less than 30%, less than 25%, less than 20%, less than 10%, less than 9%, less than 8%, less than 7%, less than 6%, less than 5%, less than 4%, less than 3%, less than 2%, less than 1%, less than 0.5%, less than 0.1%, less than 0.01%, less than 0.001%, or less than 0.0001% of an initial level, which may, for example, be a baseline level of enzyme activity.

The terms “condition,” “disease,” and “disorder” are used interchangeably.

### Proliferative Disease

A “proliferative disease” refers to a disease that occurs due to abnormal growth or extension by the multiplication of cells (Walker, Cambridge Dictionary of Biology; Cambridge University Press. Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, angiogenesis, inflammatory diseases, and autoimmune diseases.

The term “angiogenesis” refers to the physiological process through which new blood vessels form from pre-existing vessels. Angiogenesis is distinct from vasculogenesis, which is the de novo formation of endothelial cells from mesoderm cell precursors. The first vessels in a developing embryo form through vasculogenesis, after which angiogenesis is responsible for most blood vessel growth during normal or abnormal development. Angiogenesis is a vital process in growth and development, as well as in wound healing and in the formation of granulation tissue. However, angiogenesis is also a fundamental step in the transition of tumors from a benign state to a malignant one, leading to the use of angiogenesis inhibitors in the treatment of cancer. Angiogenesis may be chemically stimulated by angiogenic proteins, such as growth factors (e.g., VEGF). “Pathological angiogenesis” refers to abnormal (e.g., excessive or insufficient) angiogenesis that amounts to and/or is associated with a disease.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chrondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a class of diseases characterized by the development of abnormal cells that proliferate uncontrollably and have the ability to infiltrate and destroy normal body tissues. See e.g., Stedman's Medical Dictionary, 25th ed.: Hensyl ed.; Williams & Wilkins: Philadelphia, 1990. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; ocular cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL. T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type I or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer, vaginal cancer; and vulvar cancer (e.g., Paget's disease of the vulva).

### Anti-Cancer Agents

Anti-cancer agents encompass biotherapeutic anti-cancer agents as well as chemotherapeutic agents.

Exemplary biotherapeutic anti-cancer agents include, but are not limited to, interferons, cytokines (e.g., tumor necrosis factor, interferon α, interferon γ), vaccines, hematopoietic growth factors, monoclonal serotherapy, immunostimulants and/or immunomodulatory agents (e.g., IL-1, 2, 4, 6, or 12), immune cell growth factors (e.g., GM-CSF) and antibodies (e.g., HERCEPTIN (trastuzumab), T-DM1, AVASTIN (bevacizumab), ERBITUX (cetuximab), VECTIBIX (panitumumab), RITUXAN (rituximab), BEXXAR (tositumomab)).

Exemplary chemotherapeutic agents include, but are not limited to, anti-estrogens (e.g., tamoxifen, raloxifene, and megestrol). LHRH agonists (e.g., goserelin and leuprolide), anti-androgens (e.g., flutamide and bicalutamide), photodynamic therapies (e.g., verteporfin (BPD-MA), phthalocyanine, photosensitizer Pc4, and demethoxy-hypocrellin A (2BA-2-DMHA)), nitrogen mustards (e.g., cyclophosphamide, ifosfamide, trofosfamide, chlorambucil, estramustine, and melphalan), nitrosoureas (e.g., carmustine (BCNU) and lomustine (CCNU)), alkylsulphonates (e.g., busulfan and treosulfan), triazenes (e.g., dacarbazine, temozolomide), platinum containing compounds (e.g., cisplatin, carboplatin, oxaliplatin), vinca alkaloids (e.g., vincristine, vinblastine, vindesine, and vinorelbine), taxoids (e.g., paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., '2′-paclitaxel methyl 2-glucopyranosyl succinate; docetaxel, taxol), epipodophyllins (e.g., etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g., mycophenolic acid, tiazofurin, ribavirin, and EICAR), ribonucleotide reductase inhibitors (e.g., hydroxyurea and deferoxamine), uracil analogs (e.g., 5-fluorouracil (5-FU), floxuridine, doxifluridine, raltitrexed, tegafur-uracil, capecitabine), cytosine analogs (e.g., cytarabine (ara C), cytosine arabinoside, and fludarabine), purine analogs (e.g., mercaptopurine and Thioguanine), Vitamin D3 analogs (e.g., EB 1089, CB 1093, and KH 1060), isoprenylation inhibitors (e.g., lovastatin), dopaminergic neurotoxins (e.g., 1-methyl-4-phenylpyridinium ion), cell cycle inhibitors (e.g., staurosporine), actinomycin (e.g., actinomycin D, dactinomycin), bleomycin (e.g., bleomycin A2, bleomycin B2, peplomycin), anthracycline (e.g., daunorubicin, doxorubicin, pegylated liposomal doxorubicin, idarubicin, epirubicin, pirarubicin, zorubicin, mitoxantrone), MDR inhibitors (e.g., verapamil), Ca²⁺ ATPase inhibitors (e.g., thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxanib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CC1-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbazine, prednisolone, dexamethasone, campothecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

As used herein, the term “immunotherapy” refers to refers to any chemical or biological agent with therapeutic usefulness in the treatment of diseases characterized by abnormal cell growth by promoting, preserving, or increasing the activity of immune cells. Immunotherapies include immune checkpoint inhibitors, T-cell transfer therapy (e.g., CAR-T therapies), antibody therapies, treatment vaccines, and immune system modulators.

Immune checkpoint inhibitors inhibit one or more immune checkpoint proteins. The immune system has multiple inhibitory pathways that are critical for maintaining self-tolerance and modulating immune responses. For example, in T-cells, the amplitude and quality of response is initiated through antigen recognition by the T-cell receptor and is regulated by immune checkpoint proteins that balance co-stimulatory and inhibitory signals. In some embodiments, a subject or patient is treated with at least one inhibitor of an immune checkpoint protein. As used herein, “immune checkpoint protein” refers to a protein which, when active, exhibits an inhibitory effect on immune activity, e.g., T cell activity. Exemplary immune checkpoint proteins include PD-1 (e.g., NCBI Gene ID: 5133); PD-L1 (e.g., NCBI Gene ID: 29126); PD-L2 (e.g., NCBI Gene ID: 80380); TIM-3 (e.g., NCBI Gene ID: 84868); CTLA4 (e.g., NCBI Gene ID: 1493); TIGIT (e.g., NCBI Gene ID: 201633); KIR (e.g., NCBI Gene ID: 3811); LAG3 (e.g., NCBI Gene ID: 3902); DD1-α (e.g., NCBI Gene ID: 64115); A2AR (e.g., NCBI Gene ID: 135); B7-H3 (e.g., NCBI Gene ID: 80381); B7-H4 (e.g., NCBI Gene ID: 79679); BTLA (e.g., NCBI Gene ID: 151888); IDO (e.g., NCBI Gene ID: 3620); TDO (e.g., NCBI Gene ID: 6999); HVEM (e.g., NCBI Gene ID: 8764); GAL9 (e.g., NCBI Gene ID: 3965); 2B4 (belongs to the CD2 family of molecules and is expressed on all NK, γδ, and memory CD8+(αβ)T cells) (e.g., NCBI Gene ID: 51744); CD160 (also referred to as BY55) (e.g., NCBI Gene ID: 11126); and various B-7 family ligands. B7 family ligands include, but are not limited to, B7-1, B7-2, B7-DC, B7-H1, B7-H2, B7-H3, B7-H4, B7-H5, B7-H6 and B7-H7.

Non-limiting examples of immune checkpoint inhibitors (with checkpoint targets and manufacturers noted in parentheses) include: MGA271 (B7-H3: MacroGenics); ipilimumab (CTLA-4; Bristol Meyers Squibb); pembrolizumab (PD-1, Merck); nivolumab (PD-1; Bristol Meyers Squibb); atezolizumab (PD-L1; Genentech); galiximab (B7.1; Biogen); IMP321 (LAG3: Immuntep); BMS-986016 (LAG3; Bristol Meyers Squibb); SMB-663513 (CD137; Bristol-Meyers Squibb); PF-05082566 (CD137; Pfizer); IPH2101 (KIR; Innate Pharma); KW-0761 (CCR4; Kyowa Kirin); CDX-1127 (CD27; CellDex); MEDI-6769 (O×40; MedImmune); CP-870,893 (CD40; Genentech); tremelimumab (CTLA-4; Medimmune); pidilizumab (PD-1; Medivation); MPDL3280A(PD-L1; Roche); MEDI4736 (PD-L1; AstraZeneca); MSB0010718C (PD-L1; EMD Serono); AUNP12 (PD-1; Aurigene); avelumab (PD-L1; Merck); durvalumab (PD-L1; Medimmune); IMP321, a soluble Ig fusion protein (Brignone et al., 2007, J. Immunol. 179:42024211); the anti-B7-H₃ antibody MGA271 (Loo et al., 2012, Clin. Cancer Res. July 15 (18) 3834); TIM3 (T-cell immunoglobulin domain and mucin domain 3) inhibitors (Fourcade et al., 2010, J. Exp. Med. 207:2175-86 and Sakuishi et al., 2010, J. Exp. Med. 207:2187-94); anti-CTLA-4 antibodies described in U.S. Pat. Nos. 5,811,097; 5,811.097; 5,855,887; 6,051,227; 6,207,157; 6,682,736; 6,984,720; and 7,605,238; tremelimumab, (ticilimumab, CP-675,206); ipilimumab (also known as 10D1, MDX-DOO); PD-1 and PD-L1 blockers described in U.S. Pat. Nos. 7,488,802; 7,943,743; 8,008,449; 8,168,757; 8,217,149, and PCT Published patent application Nos: WO03042402, WO2008156712, WO2010089411, WO2010036959, WO2011066342, WO2011159877, WO2011082400, and WO2011161699; nivolumab (MDX 1106, BMS 936558, ONO 4538); lambrolizumab (MK-3475 or SCH 900475); CT-01; AMP-224; and BMS-936559 (MDX-1105-01). The foregoing references are incorporated by reference herein in their entireties.

### Inflammatory Disease or Condition

The terms “inflammatory disease” and “inflammatory condition” are used interchangeably herein, and refer to a disease or condition caused by, resulting from, or resulting in inflammation. Inflammatory diseases and conditions include those diseases, disorders or conditions that are characterized by signs of pain (dolor, from the generation of noxious substances and the stimulation of nerves), heat (calor, from vasodilatation), redness (rubor, from vasodilatation and increased blood flow), swelling (tumor, from excessive inflow or restricted outflow of fluid), and/or loss of function (functio laesa, which can be partial or complete, temporary or permanent. Inflammation takes on many forms and includes, but is not limited to, acute, adhesive, atrophic, catarrhal, chronic, cirrhotic, diffuse, disseminated, exudative, fibrinous, fibrosing, focal, granulomatous, hyperplastic, hypertrophic, interstitial, metastatic, necrotic, obliterative, parenchymatous, plastic, productive, proliferous, pseudomembranous, purulent, sclerosing, seroplastic, serous, simple, specific, subacute, suppurative, toxic, traumatic, and/or ulcerative inflammation. The term “inflammatory disease” may also refer to a dysregulated inflammatory reaction that causes an exaggerated response by macrophages, granulocytes, and/or T-lymphocytes leading to abnormal tissue damage and/or cell death. In some embodiments, the inflammatory disease is an acute or chronic inflammatory condition. In some embodiments, the inflammatory disease results from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis. Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

Additional exemplary inflammatory conditions include, but are not limited to, inflammation associated with acne, anemia (e.g., aplastic anemia, hemolytic autoimmune anemia), asthma, arteritis (e.g., polyarteritis, temporal arteritis, periarteritis nodosa. Takayasu's arteritis), arthritis (e.g., crystalline arthritis, osteoarthritis, psoriatic arthritis, gouty arthritis, reactive arthritis, rheumatoid arthritis and Reiter's arthritis), ankylosing spondylitis, amylosis, amyotrophic lateral sclerosis, autoimmune diseases, allergies or allergic reactions, atherosclerosis, bronchitis, bursitis, chronic prostatitis, conjunctivitis, Chagas disease, chronic obstructive pulmonary disease, cermatomyositis, diverticulitis, diabetes (e.g., type I diabetes mellitus, Type II diabetes mellitus), a skin condition (e.g., psoriasis, eczema, burns, dermatitis, pruntus (itch)), endometriosis, Guillain-Barre syndrome, infection, ischemic heart disease, Kawasaki disease, glomerulonephritis, gingivitis, hypersensitivity, headaches (e.g., migraine headaches, tension headaches), ileus (e.g., postoperative ileus and ileus during sepsis), idiopathic thrombocytopenic purpura, interstitial cystitis (painful bladder syndrome), gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischemic colitis, diversion colitis. Behcet's syndrome, indeterminate colitis) and inflammatory bowel syndrome (IBS)), lupus, multiple sclerosis, morphea, myasthenia gravis, myocardial ischemia, nephrotic syndrome, pemphigus vulgaris, pernicious anemia, peptic ulcers, polymyositis, primary biliary cirrhosis, neuroinflammation associated with brain disorders (e.g., Parkinson's disease, Huntington's disease, and Alzheimer's disease), prostatitis, chronic inflammation associated with cranial radiation injury, pelvic inflammatory disease, reperfusion injury, regional enteritis, rheumatic fever, systemic lupus erythematosus, scleroderma, sarcoidosis, spondyloarthropathies, Sjogren's syndrome, thyroiditis, transplantation rejection, tendonitis, trauma or injury (e.g., frostbite, chemical irritants, toxins, scarring, burns, physical injury), vasculitis, vitiligo and Wegener's granulomatosis. In certain embodiments, the inflammatory disorder is selected from arthritis (e.g., rheumatoid arthritis), inflammatory bowel disease, inflammatory bowel syndrome, asthma, psoriasis, endometriosis, interstitial cystitis and prostatitis. In certain embodiments, the inflammatory condition is an acute inflammatory condition (e.g., for example, inflammation resulting from infection). In certain embodiments, the inflammatory condition is a chronic inflammatory condition (e.g., conditions resulting from asthma, arthritis and inflammatory bowel disease). The compounds may also be useful in treating inflammation associated with trauma and non-inflammatory myalgia. The compounds disclosed herein may also be useful in treating inflammation associated with cancer.

Exemplary anti-inflammatory agents include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; opiates (e.g. endorphins, enkephalins, and dynorphin), steroids, analgesics, barbiturates, oxycodone, morphine, lidocaine, and the like. In some embodiments, the anti-inflammatory agent is a steroid (e.g., a corticosteroid or glucocorticoid); a calcineurin inhibitor (e.g. cyclosporine, tacrolimus, pimecrolimus, or FK506); an mTOR inhibitor (e.g., everolimus, temsirolimus, rapamycin, deforolimus, TOP216, OSI-027, TAFA93, nab-rapamycin, tacrolimus, biolimus, CI-779, ABT-578, AP-23675, BEZ-235, QLT0447, ABI-009, BC-210, salirasib, AP-23841, AP-23573, KU-0059475, 32-deoxorapamycin, 16-pent-2-ynyloxy-32deoxorapamycin, 16-pent-2-ynyloxy-32 (S or R)-dihydrorapamycin, 16-pent-2-ynyloxy-32 (S or R)-dihydro-40-O(2-hydroxyethyl)-rapamycin, 40-O-(2-hydroxyethyl)rapamycin, 32-deoxorapamycin; 16-pent-2-ynyloxy-32(S)dihydrorapamycin: socalledrapalogs; AP23464; PI-103, PP242, PP30, Torin1; and derivatives or pharmaceutically acceptable salts thereof as well as and compounds described in, e.g. U.S. Patent Publications 2011/0178070; 2011/0021515; 2007/0112005; 2011/0054013; International Patent Publications WO98/02441; WO01/14387; WO99/15530; WO07/135411: WO03/64383; WO96/41807; WO95/16691; WO94/09010; European Patent No. EP1880723; and U.S. Pat. Nos. 8,163,775; 6,329,386; 6,200,985; 6,117,863; 6,015,815; 6,015,809; 6.004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300, the contents of which are incorporated by reference herein.

As used herein, “inflammatory response” refers to one or more aspects or components of inflammation. As used herein, “inflammation” refers to the complex biological response to harmful stimuli, such as pathogens, damaged cells, or irritants. Inflammation is a protective attempt by the organism to remove the injurious stimuli as well as initiate the healing process for the tissue. Accordingly, the term “inflammation” includes any cellular process that leads to the production of pro-inflammatory cytokines, inflammation mediators and/or the related downstream cellular events resulting from the actions of the cytokines thus produced, for example, fever, fluid accumulation, swelling, abscess formation, and cell death.

### Immune Disorder

Immune disorders, such as auto-immune disorders, include, but are not limited to, arthritis (including rheumatoid arthritis, spondyloarthropathies, gouty arthritis, degenerative joint diseases such as osteoarthritis, systemic lupus erythematosus, Sjogren's syndrome, ankylosing spondylitis, undifferentiated spondylitis, Behcet's disease, haemolytic autoimmune anemias, multiple sclerosis, amyotrophic lateral sclerosis, amylosis, acute painful shoulder, psoriatic, and juvenile arthritis), asthma, atherosclerosis, osteoporosis, bronchitis, tendonitis, bursitis, skin condition (e.g., psoriasis, eczema, burns, dermatitis, pruritus (itch)), enuresis, eosinophilic disease, gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)), and disorders ameliorated by a gastroprokinetic agent (e.g., ileus, postoperative ileus and ileus during sepsis; gastroesophageal reflux disease (GORD, or its synonym GERD); eosinophilic esophagitis, gastroparesis such as diabetic gastroparesis; food intolerances and food allergies and other functional bowel disorders, such as non-ulcerative dyspepsia (NUD) and non-cardiac chest pain (NCCP, including costo-chondritis)).

### Autoimmune Diseases

An “autoimmune disease” refers to a disease arising from an inappropriate immune response of the body of a subject against substances and tissues normally present in the body. In other words, the immune system mistakes some part of the body as a pathogen and attacks its own cells. This may be restricted to certain organs (e.g., in autoimmune thyroiditis) or involve a particular tissue in different places (e.g., Goodpasture's disease which may affect the basement membrane in both the lung and kidney). The treatment of autoimmune diseases is typically with immunosuppression, e.g., medications which decrease the immune response. Exemplary autoimmune diseases include, but are not limited to, glomerulonephritis, Goodpasture's syndrome, necrotizing vasculitis, lymphadenitis, peri-arteritis nodosa, systemic lupus erythematosis, rheumatoid arthritis, psoriatic arthritis, psoriasis, ulcerative colitis, systemic sclerosis, dermatomyositis/polymyositis, anti-phospholipid antibody syndrome, scleroderma, pemphigus vulgaris, ANCA-associated vasculitis (e.g., Wegener's granulomatosis, microscopic polyangiitis), uveitis, Sjogren's syndrome, Crohn's disease, Reiter's syndrome, ankylosing spondylitis, Lyme disease, Guillain-Barre syndrome, Hashimoto's thyroiditis, and cardiomyopathy.

### Mix of Immune and Inflammatory

Some diseases are a mixture of immune and inflammatory disorders. In certain embodiments, the mixture of inflammatory and the immune disorder is a gastrointestinal disorder. In some embodiments, the gastrointestinal disorder is selected from gastrointestinal disorder (e.g., selected from peptic ulcers, regional enteritis, diverticulitis, gastrointestinal bleeding, eosinophilic gastrointestinal disorders (e.g., eosinophilic esophagitis, eosinophilic gastritis, eosinophilic gastroenteritis, eosinophilic colitis), gastritis, diarrhea, gastroesophageal reflux disease (GORD, or its synonym GERD), inflammatory bowel disease (IBD) (e.g., Crohn's disease, ulcerative colitis, collagenous colitis, lymphocytic colitis, ischaemic colitis, diversion colitis, Behcet's syndrome, indeterminate colitis) and inflammatory bowel syndrome (IBS)). In certain embodiments, the gastrointestinal disorder is inflammatory bowel disease (IBD).

In certain embodiments, the inflammatory condition and/or immune disorder is a skin condition. In some embodiments, the skin condition is pruritus (itch), psoriasis, eczema, burns or dermatitis. In certain embodiments, the skin condition is psoriasis. In certain embodiments, the skin condition is pruritis.

### DETAILED DESCRIPTION OF CERTAIN EMBODIMENTS

Interleukin-4 (IL-4) is an important immunoregulatory cytokine involved in T cell maturation, B cell activation, and macrophage polarization. Dysregulated IL-4 signaling contributes to a number of immune-mediated diseases such as allergic inflammation, cancer, and autoimmunity. The clinical use and indication expansion of the anti-IL-4Rα antibody dupilumab has made IL-4 signaling an attractive target.

Previously, the discovery of the first small-molecule IL-4 inhibitor, named Nico-52, was reported.⁴⁸ Presented herein is the determination of structure-activity relationships around the core scaffold of Nico-52 to characterize and improve the potency and selectivity of the compound. Certain analogs featured structural changes to the p-fluorophenyl group ranging from submicromolar to double-digit nanomolar potency. Two analogs showed selective binding to IL-4 over other related cytokines in thermal shift assays along with more potent inhibition of IL-4 over IL-13 in a HEK blue IL-4/IL-13 reporter assay. It was established that certain analogs inhibit both type I and type II IL-4 receptor signaling in Ramos B lymphocytes and THP-1 monocytes respectively. Finally, promising in vitro ADME/T properties were determined for certain analogs. Analogs were evaluated in a HEK-Blue IL-4/IL-13 reporter assay at a single dose of 10 μM.

Prior efforts directed to treating conditions associated with IL-4-mediated inflammation have generally been directed to inhibiting the protein-protein interactions between cytokines and their receptors, typically the domain of therapeutic antibodies. One current clinical therapeutic for IL-4 inhibition is the anti-IL-4Rα antibody dupilumab, which was first approved for treating atopic dermatitis and inhibits IL-4 activity by binding to the IL-4 membrane receptor (IL-4Rα). The antibody is now approved for treating a wide range of inflammatory conditions including moderate-to-severe asthma, eosinophilic esophagitis, prurigo nodularis, and chronic rhinosinusitis with nasal polyposis.⁹⁻¹¹ Dupilumab is also currently being clinically investigated for treating metastatic NSCLC in conjugation with PD-L after the observation that anti-IL-4 treatment reduced tumor burden in murine models.^{12,13} Results from a Phase 3 clinical trial assessing dupilumab for Chronic Obstructive Pulmonary Disease (COPD) are expected soon, highlighting yet another indication (Clinical Trial Identifier NCT03930732).

Monoclonal antibody (mAbs) therapeutics face limitations in administration routes, primarily due to their large size¹⁴ and hydrophilic nature as glycoproteins.¹⁵ These characteristics contribute to their gastrointestinal instability and non-specific elimination by the reticuloendothelial system, restricting their route of administration to intravenous delivery.¹⁶⁻¹⁸ These mAbs can pose toxicity issues arising from target-related effects and immunogenicity (target-independent).¹⁹⁻²² Immunogenic responses against mAbs can potentially result in decreased therapeutic efficacy.²³ Maintaining mAbs therapy presents challenges due to characteristics such as long half-life, limited tissue residence time, and slower progression to peak concentration.²⁴⁻²⁷ In contrast, small molecules, characterized by low molecular weight, rapid absorption, good bioavailability, lack of immunogenicity, and rapid metabolism, offer advantages that enable oral delivery, consistent dosing, and precise control over the timing of therapy.^{14, 23-28}

Recent advances in screening technologies and assays for interrogating protein-protein interactions have renewed interest in directly disrupting cytokine binding with small molecules.²⁹ DiCE Molecules and LEO Pharma are pursuing small molecule or macrocyclic IL-17A inhibitors to rival Cosentyx, with both companies in early Phase 1 clinical trials.³⁰⁻³² Similarly, SPD304, initially an anti-TNFα inhibitor although with low potency and poor physiochemical properties, paved the way for the discovery of a benpyrine compound as an anti-TNFα inhibitor.^{29,33-39} SAR441566 is a TNFα inhibitor that is currently in a Phase 2 Clinical Trial for Rheumatoid Arthritis (NCT06073093). Moreover, a combination of high throughput screening, virtual screening, and biophysical & biochemical assays have been utilized to discover small molecule inhibitors for the soluble cytokines IL-1β, IL-2, IL-33(sST2), IL-36γ, IL-15, and dimeric IL-23.^{29,40-47}

The discovery of the first small-molecule IL-4 inhibitor named Nico-52 was recently reported.⁴⁸ Herein, it was determined structure-activity relationships around the core scaffold of Nico-52 to characterize and improve the potency and selectivity of the compound, 56 analogs were evaluated in a HEK-Blue IL-4/IL-13 reporter assay at a single dose of 10 μM. Analogs that inhibited IL-4 signaling >80% were then tested for dose-dependent responses to better characterize their potency, with certain analogs exhibiting potencies in the nanomolar range. Certain potent analogs featured structural changes to the p-fluorophenyl substituent whereas the amino nicotinonitrile core. Differential scanning fluorimetry (DSF) screening against related cytokines suggested that Nico-52 and improved analogs are selective for binding to IL-4 over other related proteins. It was further established that Nico-52 and improved analogs inhibit both type I and II receptor complex signaling, inhibition of murine IL-4 signaling by Nico-52 and determined in vitro ADME/T properties of certain analogs.

The compounds, compositions, methods and kits provided herein are also useful for the targeting of additional therapeutic agents to sites of IL-4 activity. For example, compounds that bind but do not substantially inhibit IL-4 have utility as tool compounds and/or as non-inhibiting targeting agents that can enable co-localization of payloads within specific cytokine-rich environments. Accordingly, in another aspect, provided herein are therapeutic compositions comprising at least one IL-4 inhibitor or binder as described herein and at least a second therapeutic molecule.

### Compounds

In one aspect, provided herein, compound of formula (I).

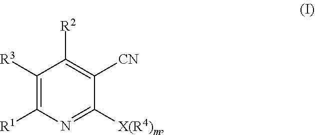

or a pharmaceutically acceptable salt, solvate, hydrate, polymorph, co-crystal, tautomer, stereoisomer, isotopically labeled compound, or prodrug thereof, wherein:
X is N, S, O, or halo,
each of R¹ and R² is independently optionally substituted alkyl, optionally substituted aryl, optionally substituted heteroaryl, optionally substituted heterocyclyl, optionally substituted amino, optionally substituted alkoxy, hydroxyl, or optionally substituted carbocyclyl, wherein at least one of R¹ and R² is optionally substituted cyclopropyl, optionally substituted cyclobutyl, or optionally substituted bicyclopentanyl;
R³ is hydrogen or optionally substituted alkyl,
each R⁴ is independently hydrogen, optionally substituted alkyl, a nitrogen protecting group when appended to a nitrogen, an oxygen protecting group when appended to an oxygen, a sulfur protecting group when appended to a sulfur, or, where X is N, two instances of R⁴, together with the nitrogen atom to which they are attached, form an optionally substituted heterocyclyl or heteroaryl; and
m is 0, 1, or 2.

In some embodiments, m is 0. In some embodiments, m is 1. In certain embodiments, m is 2.

In certain embodiments, the compound is of Formula (I-a), (I-b), (1-c), or (1-d):

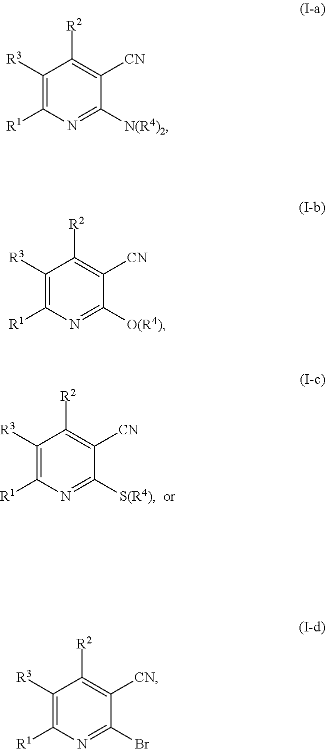

wherein each R⁴ is independently hydrogen, optionally substituted C₁₋₆ alkyl, or two instances of R⁴, together with the atom to which they are attached, form an optionally substituted heterocyclyl or heteroaryl, and R³ is hydrogen or optionally substituted C₁₋₆ alkyl.

In certain embodiments, the compound is of Formula (I-a)

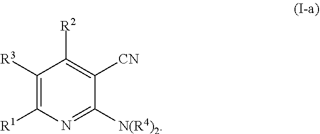

In certain embodiments, the compound is of Formula (I-b)

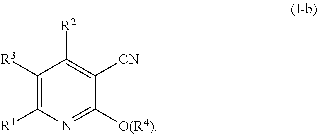

In certain embodiments, the compound is of Formula (I-c)

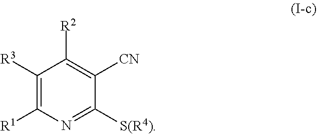

In certain embodiments, the compound is of Formula (I-d) or

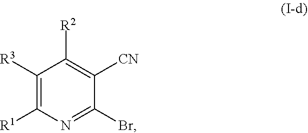

In certain embodiments, the compound is of Formula (I-a), at least one of R¹ and R² is optionally substituted alkyl, optionally substituted aryl, optionally substituted heteroaryl, optionally substituted heterocyclyl, optionally substituted amino, optionally substituted alkoxy, hydroxyl, or optionally substituted carbocyclyl, R³ is hydrogen or optionally substituted alkyl, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-a), at least one of R¹ and R² is optionally substituted aryl, optionally substituted heteroaryl, optionally substituted heterocyclyl, or optionally substituted carbocyclyl, R³ is hydrogen or optionally substituted alkyl, each R⁴ is independently hydrogen, optionally substituted C₁₋₆alkyl, or two instances of R⁴, together with the atom to which they are attached, form an optionally substituted heterocyclyl or heteroaryl.

In certain embodiments, the compound is of Formula (I-a), at least one of R¹ and R² is optionally substituted aryl, optionally substituted heteroaryl, optionally substituted heterocyclyl, or optionally substituted carbocyclyl, R³ is hydrogen, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-a), at least one of R¹ and R² is optionally substituted aryl, optionally substituted heteroaryl, optionally substituted heterocyclyl, or optionally substituted carbocyclyl, R³ is hydrogen, each R⁴ is independently hydrogen or optionally substituted C₁₋₆ alkyl.

In certain embodiments, the compound is of Formula (I-b), at least one of R¹ and R² is optionally substituted alkyl, optionally substituted aryl, optionally substituted heteroaryl, optionally substituted heterocyclyl, optionally substituted amino, optionally substituted alkoxy, hydroxyl, or optionally substituted carbocyclyl, R³ is hydrogen or optionally substituted alkyl, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-b), at least one of R¹ and R² is optionally substituted aryl, optionally substituted heteroaryl, optionally substituted heterocyclyl, or optionally substituted carbocyclyl, R³ is hydrogen or optionally substituted alkyl, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-b), at least one of R¹ and R² is optionally substituted aryl, optionally substituted heteroaryl, optionally substituted heterocyclyl, or optionally substituted carbocyclyl, R³ is hydrogen, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-b), at least one of R¹ and R² is optionally substituted aryl, optionally substituted heteroaryl, optionally substituted heterocyclyl, or optionally substituted carbocyclyl, R³ is hydrogen, each R⁴ is independently hydrogen or optionally substituted C₁₋₆ alkyl.

In certain embodiments, the compound is of Formula (I-c), at least one of R¹ and R² is optionally substituted alkyl, optionally substituted aryl, optionally substituted heteroaryl, optionally substituted heterocyclyl, optionally substituted amino, optionally substituted alkoxy, hydroxyl, or optionally substituted carbocyclyl, R³ is hydrogen or optionally substituted alkyl, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-c), at least one of R¹ and R² is optionally substituted aryl, optionally substituted heteroaryl, optionally substituted heterocyclyl, or optionally substituted carbocyclyl, R³ is hydrogen or optionally substituted alkyl, each R⁴ is independently hydrogen, optionally substituted C₁₋₆alkyl, or two instances of R⁴, together with the atom to which they are attached, form an optionally substituted heterocyclyl or heteroaryl.

In certain embodiments, the compound is of Formula (I-c), at least one of R¹ and R² is optionally substituted aryl, optionally substituted heteroaryl, optionally substituted heterocyclyl, or optionally substituted carbocyclyl, R³ is hydrogen, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-c), at least one of R¹ and R² is optionally substituted aryl, optionally substituted heteroaryl, optionally substituted heterocyclyl, or optionally substituted carbocyclyl, R³ is hydrogen, each R⁴ is independently hydrogen or optionally substituted C₁₋₆ alkyl.

In certain embodiments, the compound is of Formula (I-d), at least one of R¹ and R² is optionally substituted alkyl, optionally substituted aryl, optionally substituted heteroaryl, optionally substituted heterocyclyl, optionally substituted amino, optionally substituted alkoxy, hydroxyl, or optionally substituted carbocyclyl, R³ is hydrogen or optionally substituted alkyl, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-d), at least one of R¹ and R² is optionally substituted aryl, optionally substituted heteroaryl, optionally substituted heterocyclyl, or optionally substituted carbocyclyl, R³ is hydrogen or optionally substituted alkyl, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-d), at least one of R¹ and R² is optionally substituted aryl, optionally substituted heteroaryl, optionally substituted heterocyclyl, or optionally substituted carbocyclyl, R³ is hydrogen, each R⁴ is independently hydrogen, optionally substituted C₁₋₆ alkyl, or two instances of R⁴, together with the atom to which they are attached, form an optionally substituted heterocyclyl or heteroaryl.

In certain embodiments, the compound is of Formula (I-d), at least one of R¹ and R² is optionally substituted aryl, optionally substituted heteroaryl, optionally substituted heterocyclyl, or optionally substituted carbocyclyl, R³ is hydrogen, each R⁴ is independently hydrogen or optionally substituted C₁₋₆ alkyl.

In some embodiments, one of R¹ and R² is optionally substituted aryl, optionally substituted heteroaryl, optionally substituted heterocyclyl, optionally substituted amino, optionally substituted alkoxy, hydroxyl, or optionally substituted carbocyclyl.

In certain embodiments, one of R¹ and R² is optionally substituted aryl, optionally substituted heteroaryl, optionally substituted heterocyclyl, or optionally substituted C₁₋₆ alkyl.

In some embodiments, one of R¹ and R² is optionally substituted aryl or optionally substituted heteroaryl.

In certain embodiments, one of R¹ and R² is optionally substituted aryl. In certain embodiments, one of R¹ and R² is optionally substituted heteroaryl. In certain embodiments, one of R¹ and R² is optionally substituted heterocyclyl. In certain embodiments, one of R¹ and R² is optionally substituted C₁₋₆ alkyl.

In certain embodiments, R¹ is optionally substituted aryl. In certain embodiments, R¹ is optionally substituted heteroaryl. In certain embodiments, R¹ is optionally substituted heterocyclyl. In certain embodiments, R¹ is optionally substituted C₁₋₆ alkyl.

In certain embodiments, R² is optionally substituted aryl. In certain embodiments, R² is optionally substituted heteroaryl. In certain embodiments, R² is optionally substituted heterocyclyl. In certain embodiments, R² is optionally substituted C₁₋₆ alkyl.

In certain embodiments, one of R¹ and R² is aryl or heteroaryl substituted with at least one of hydroxyl, alkoxyl, halogen, amino, alkyl, acyl, or heterocyclyl. In certain embodiments, one of R¹ and R² is aryl or heteroaryl substituted with at least one of hydroxyl. In certain embodiments, one of R¹ and R² is aryl or heteroaryl substituted with at least one of alkoxyl. In certain embodiments, one of R¹ and R² is aryl or heteroaryl substituted with at least one of halogen. In certain embodiments, one of R¹ and R² is aryl or heteroaryl substituted with at least one of amino. In certain embodiments, one of R¹ and R² is aryl or heteroaryl substituted with at least one of alkyl. In certain embodiments, one of R¹ and R² is aryl or heteroaryl substituted with at least one of acyl. In certain embodiments, one of R¹ and R² is aryl or heteroaryl substituted with at least one of heterocyclyl.

In certain embodiments, R¹ is aryl or heteroaryl substituted with at least one of hydroxyl, alkoxyl, halogen, amino, alkyl, acyl, or heterocyclyl. In certain embodiments. R¹ is aryl or heteroaryl substituted with at least one of hydroxyl. In certain embodiments, R¹ is aryl or heteroaryl substituted with at least one of alkoxyl. In certain embodiments, R¹ is aryl or heteroaryl substituted with at least one of halogen. In certain embodiments, R¹ is aryl or heteroaryl substituted with at least one of amino. In certain embodiments, R¹ is aryl or heteroaryl substituted with at least one of alkyl. In certain embodiments. R¹ is aryl or heteroaryl substituted with at least one of acyl. In certain embodiments, R¹ is aryl or heteroaryl substituted with at least one of heterocyclyl.

In certain embodiments, R¹ is any substituted with at least one of hydroxyl, alkoxyl, halogen, amino, alkyl, acyl, or heterocyclyl. In certain embodiments, R¹ is aryl substituted with at least one of hydroxyl. In certain embodiments, R¹ is aryl substituted with at least one of alkoxyl. In certain embodiments, R¹ is aryl substituted with at least one of halogen. In certain embodiments, R¹ is aryl substituted with at least one of amino. In certain embodiments, R¹ is aryl substituted with at least one of alkyl. In certain embodiments, R¹ is aryl substituted with at least one of acyl. In certain embodiments, R¹ is aryl substituted with at least one of heterocyclyl.

In certain embodiments, R¹ is heteroaryl substituted with at least one of hydroxyl, alkoxyl, halogen, amino, alkyl, acyl, or heterocyclyl. In certain embodiments, R¹ is heteroaryl substituted with at least one of hydroxyl. In certain embodiments, R¹ is heteroaryl substituted with at least one of alkoxyl. In certain embodiments, R¹ is heteroaryl substituted with at least one of halogen. In certain embodiments, R¹ is heteroaryl substituted with at least one of amino. In certain embodiments, R¹ is heteroaryl substituted with at least one of alkyl. In certain embodiments, R¹ is heteroaryl substituted with at least one of acyl. In certain embodiments, R¹ is heteroaryl substituted with at least one of heterocyclyl.

In certain embodiments, R² is aryl or heteroaryl substituted with at least one of hydroxyl, alkoxyl, halogen, amino, alkyl, acyl, or heterocyclyl. In certain embodiments, R² is aryl or heteroaryl substituted with at least one of hydroxyl. In certain embodiments, R² is aryl or heteroaryl substituted with at least one of alkoxyl. In certain embodiments, R² is aryl or heteroaryl substituted with at least one of halogen. In certain embodiments, R² is aryl or heteroaryl substituted with at least one of amino. In certain embodiments, R² is aryl or heteroaryl substituted with at least one of alkyl. In certain embodiments, R² is any or heteroaryl substituted with at least one of acyl. In certain embodiments, R² is aryl or heteroaryl substituted with at least one of heterocyclyl.

In certain embodiments. R² is aryl substituted with at least one of hydroxyl, alkoxyl, halogen, amino, alkyl, acyl, or heterocyclyl. In certain embodiments, R² is aryl substituted with at least one of hydroxyl. In certain embodiments, R² is aryl substituted with at least one of alkoxyl. In certain embodiments, R² is aryl substituted with at least one of halogen. In certain embodiments, R² is aryl substituted with at least one of amino. In certain embodiments, R² is aryl substituted with at least one of alkyl. In certain embodiments, R² is aryl substituted with at least one of acyl. In certain embodiments, R² is aryl substituted with at least one of heterocyclyl.

In certain embodiments, R² is heteroaryl substituted with at least one of hydroxyl, alkoxyl, halogen, amino, alkyl, acyl, or heterocyclyl. In certain embodiments. R² is heteroaryl substituted with at least one of hydroxyl. In certain embodiments, R² is heteroaryl substituted with at least one of alkoxyl. In certain embodiments, R² is heteroaryl substituted with at least one of halogen. In certain embodiments, R² is heteroaryl substituted with at least one of amino. In certain embodiments, R² is heteroaryl substituted with at least one of alkyl. In certain embodiments, R² is heteroaryl substituted with at least one of acyl. In certain embodiments, R² is heteroaryl substituted with at least one of heterocyclyl.

In some embodiments, one of R¹ and R² is optionally substituted phenyl, biphenyl, thiophenyl, naphthyl, furanyl, or pyridinyl.

In certain embodiments, one of R¹ and R² is aryl substituted with at least one of hydroxyl, alkoxyl, halogen, amino, alkyl, acyl, or heteroaryl. In certain embodiments, one of R¹ and R² is phenyl substituted with at least one of hydroxyl, alkoxyl, halogen, amino, alkyl, acyl, or heteroaryl. In certain embodiments, one of R¹ and R² is optionally substituted heteroaryl. In certain embodiments, one of R¹ and R² is heteroaryl substituted with at least one of hydroxyl, alkoxyl, halogen, amino, alkyl, acyl, or heteroaryl. In certain embodiments, one of R¹ and R² is biphenyl substituted with at least one of hydroxyl, alkoxyl, halogen, amino, alkyl, acyl, or heteroaryl. In certain embodiments, one of R¹ and R² is thiophenyl substituted with at least one of hydroxyl, alkoxyl, halogen, amino, alkyl, acyl, or heteroaryl. In certain embodiments, one of R¹ and R² is naphthyl substituted with at least one of hydroxyl, alkoxyl, halogen, amino, alkyl, acyl, or heteroaryl. In certain embodiments, one of R¹ and R² is furanyl substituted with at least one of hydroxyl, alkoxyl, halogen, amino, alkyl, acyl, or heteroaryl. In certain embodiments, one of R¹ and R² is pyridinyl substituted with at least one of hydroxyl, alkoxyl, halogen, amino, alkyl, acyl, or heteroaryl.

In some embodiments, one of R¹ and R² is phenyl substituted with hydroxyl, halogen, or alkyl. In some embodiments, one of R¹ and R² is phenyl substituted with at least one hydroxyl. In some embodiments, one of R¹ and R² is phenyl substituted with at least one halogen. In some embodiments, one of R¹ and R² is phenyl substituted with at least one alkyl.

In some embodiments, one of R¹ and R² is unsubstituted heteroaryl.

In certain embodiments, one of R¹ and R² is optionally substituted C₁₋₆ alkyl. In certain embodiments, one of R¹ and R² is optionally substituted C₁₋₃ alkyl.

In some embodiments, at least one of R¹ and R² is cyclopropyl, cyclobutyl, or bicyclopentanyl.

In certain embodiments, at least one of R¹ and R² is selected from the group consisting of:

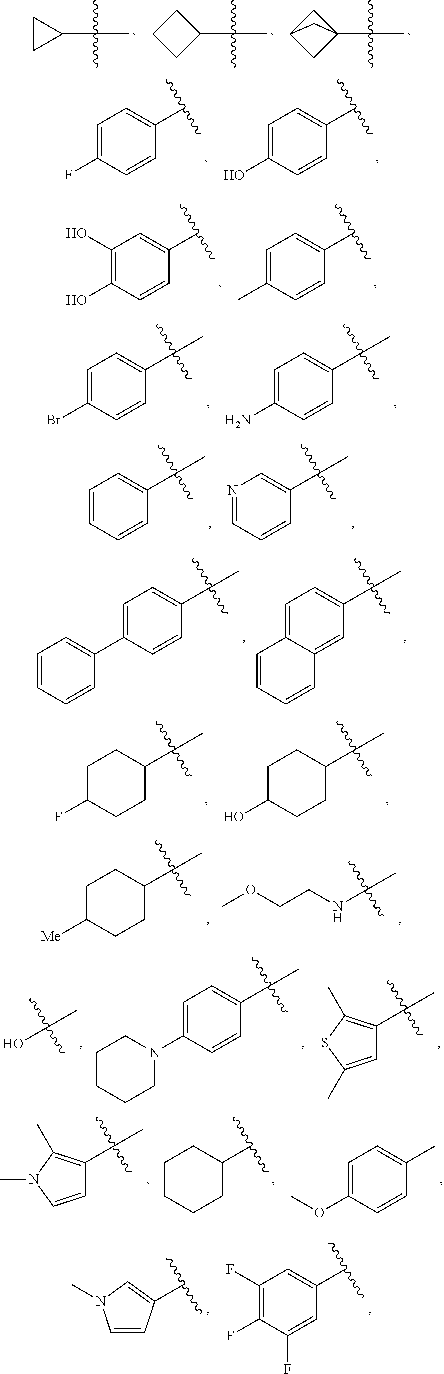

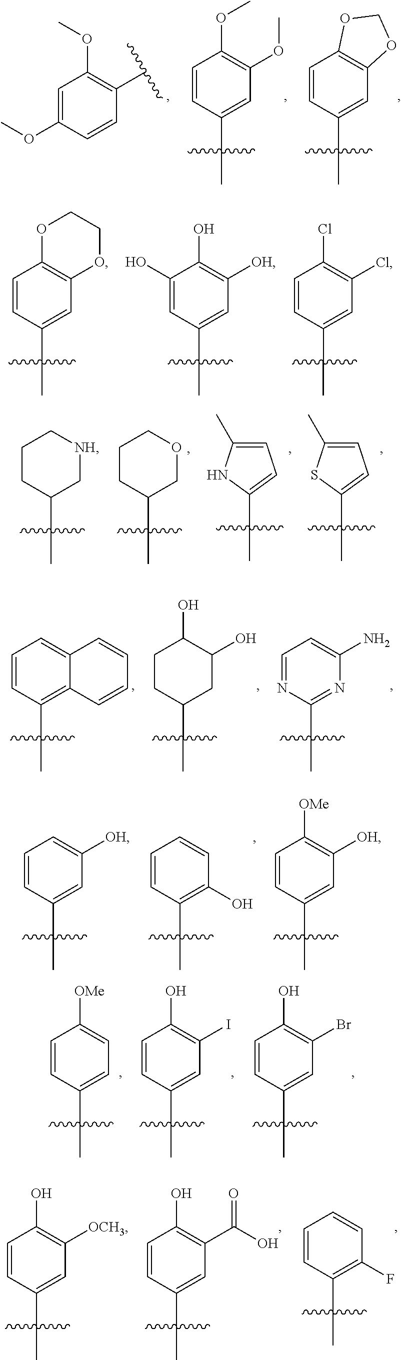

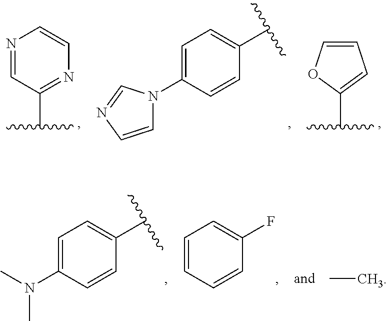

In certain embodiments. R¹ is

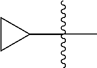 [SMILES: *C1CC1]

and R² is selected from the group consisting of:

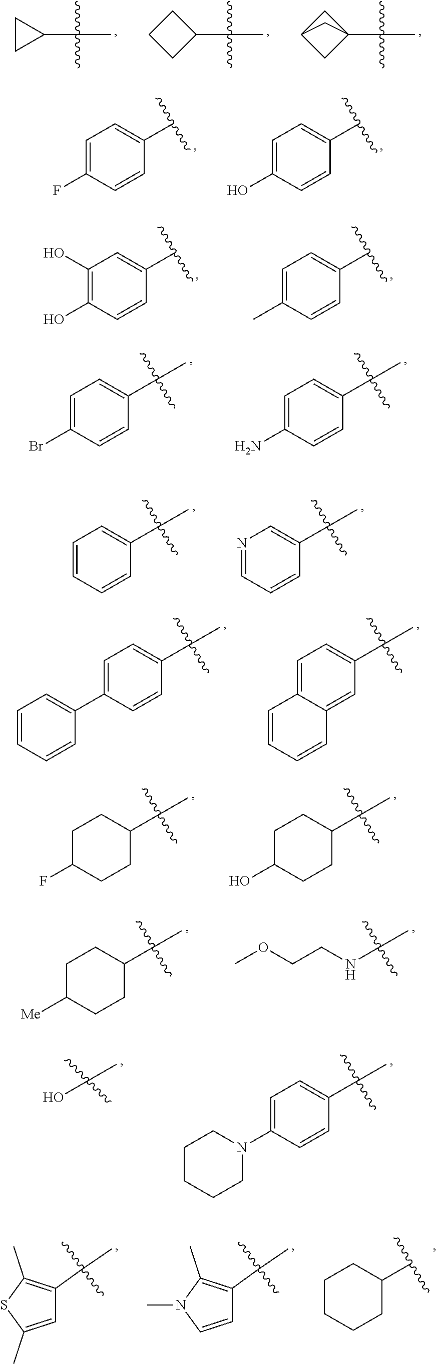

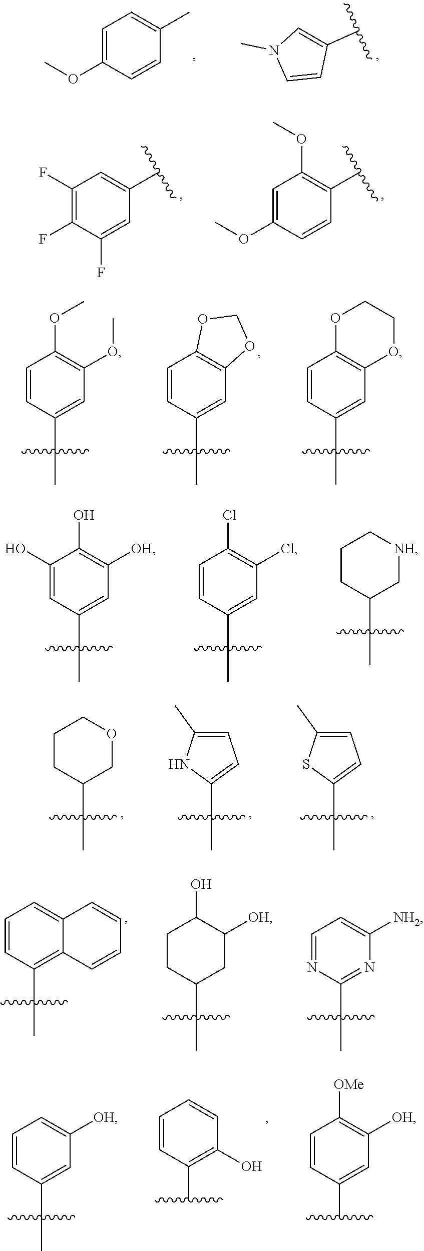

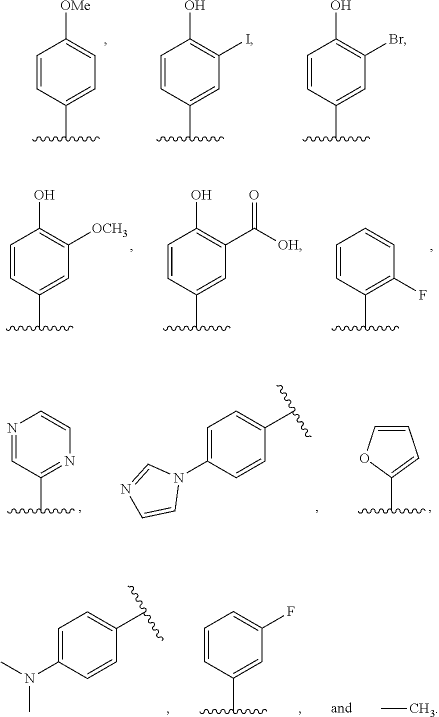

In certain embodiments, at R² is

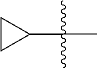 [SMILES: *C1CC1]

and R¹ is selected from the group consisting

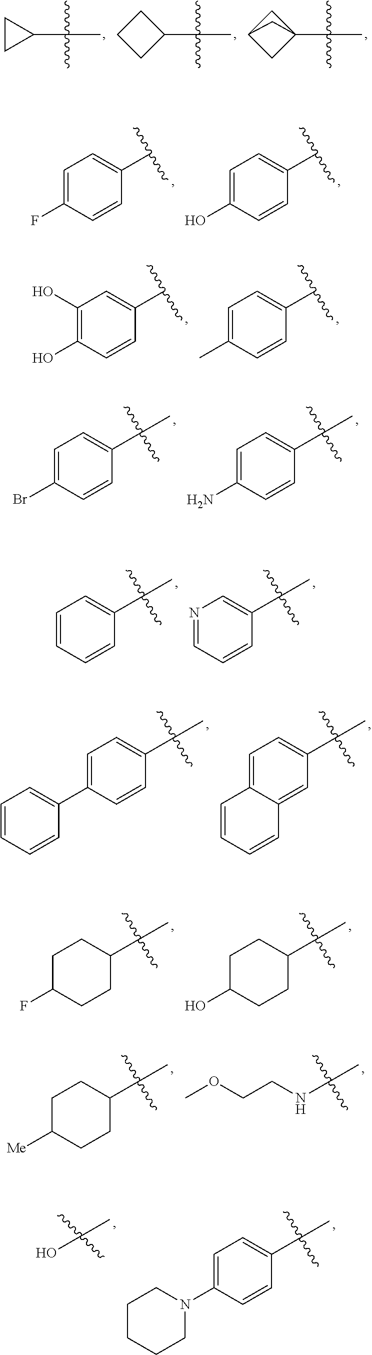

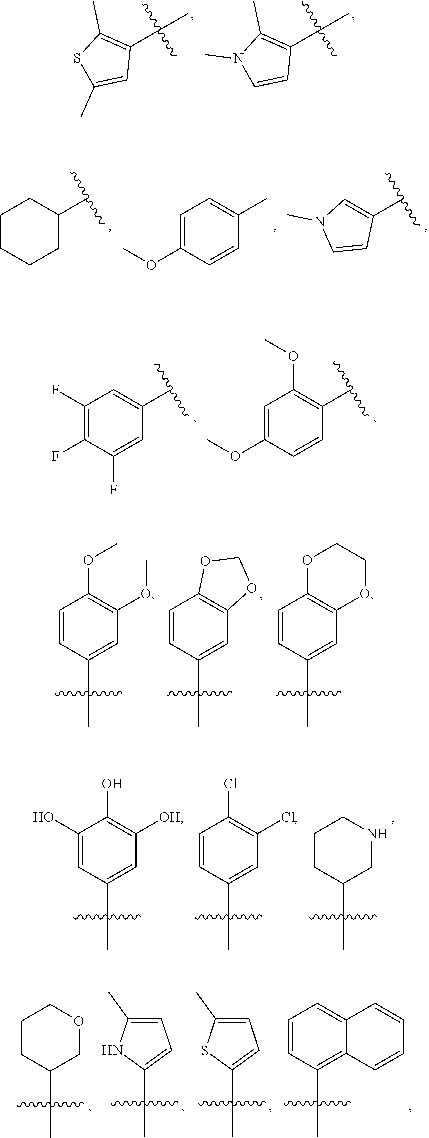

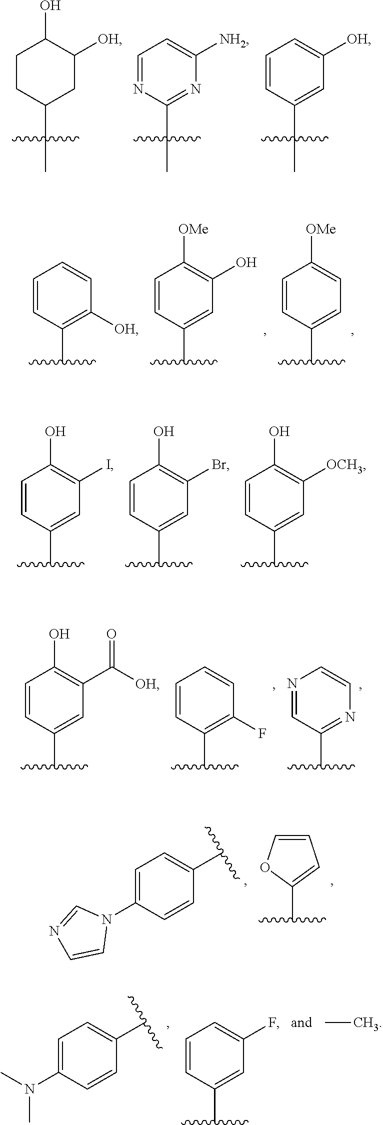

In certain embodiments, R¹ is

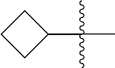 [SMILES: *C1CCC1]

and R² is selected from the group consisting

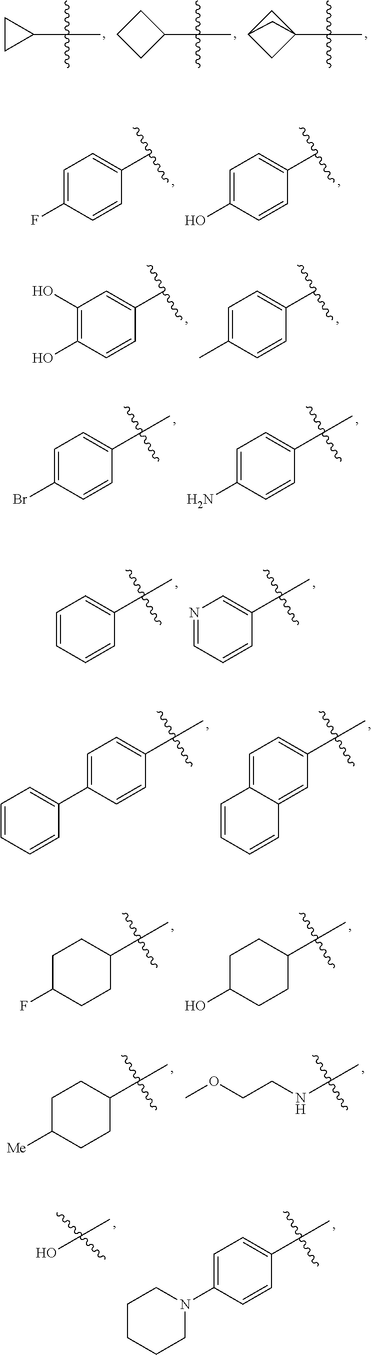

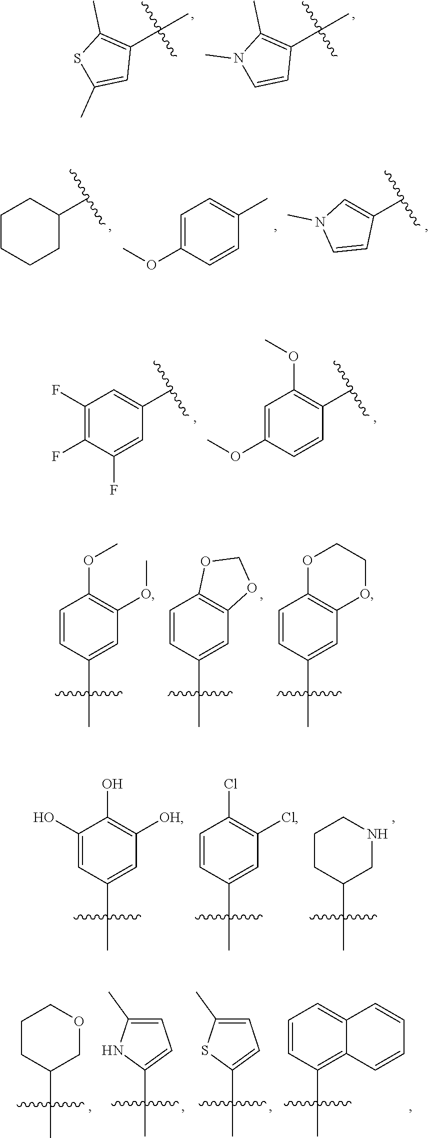

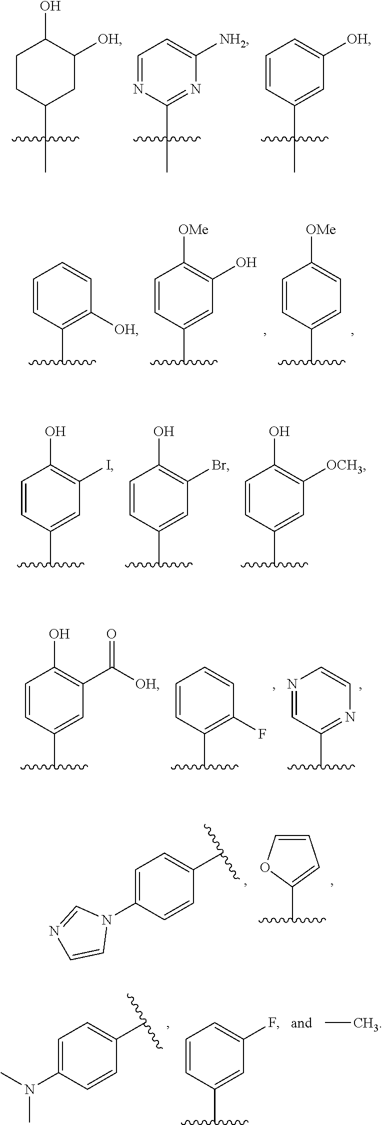

In certain embodiments, R² is

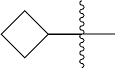 [SMILES: *C1CCC1]

and R¹ is selected from the group consisting

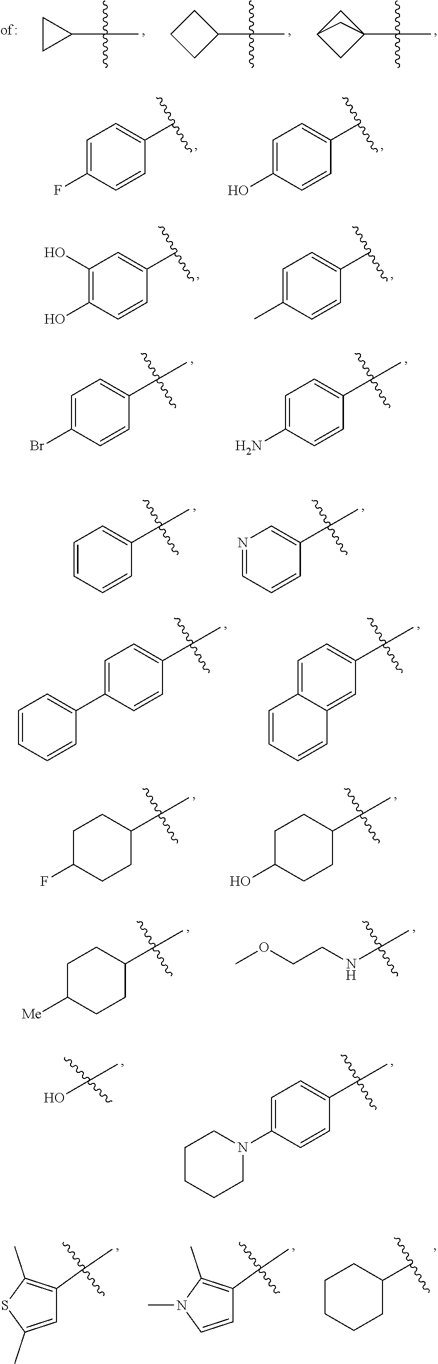

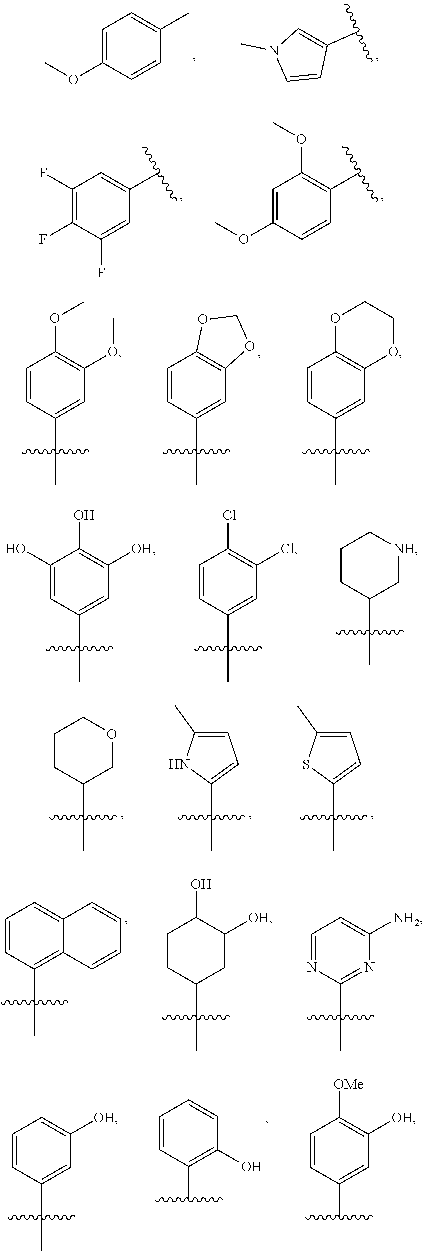

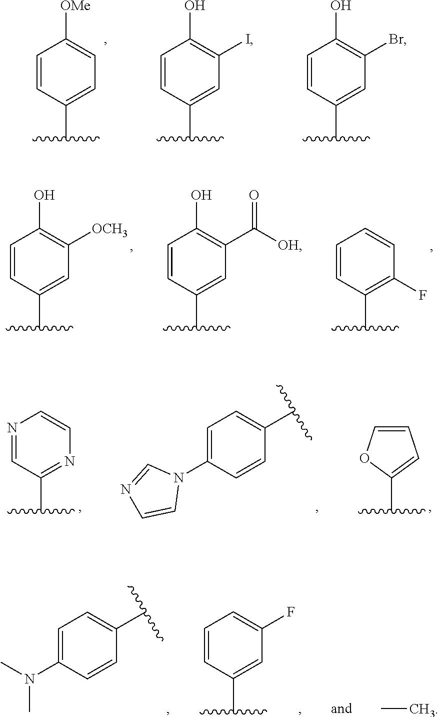

In certain embodiments, R¹ is

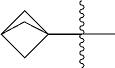 [SMILES: *C12CC(C1)C2]

and R² selected from the group consisting of:

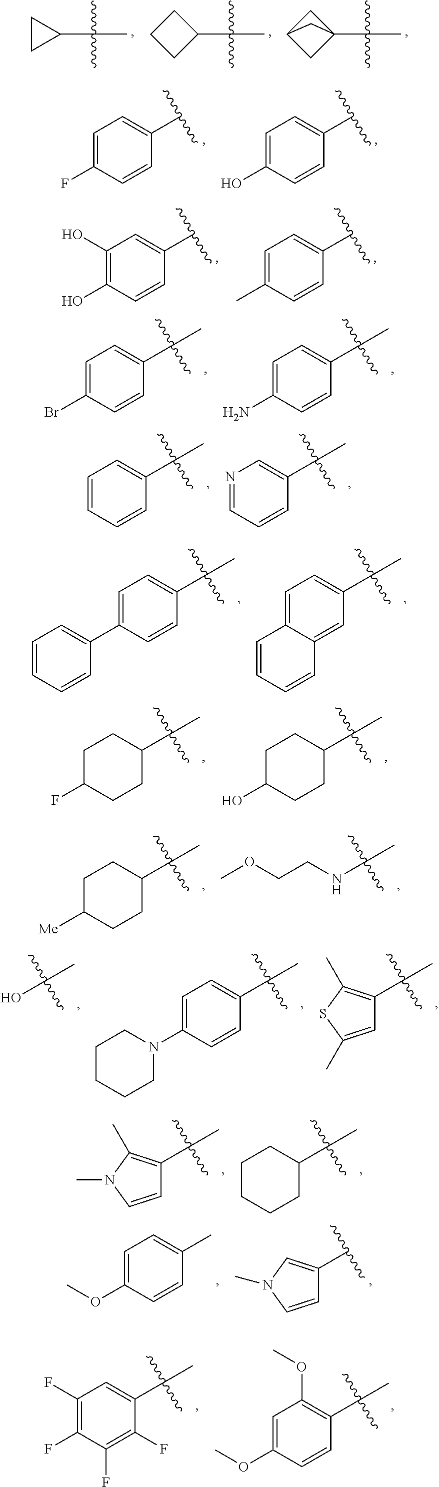

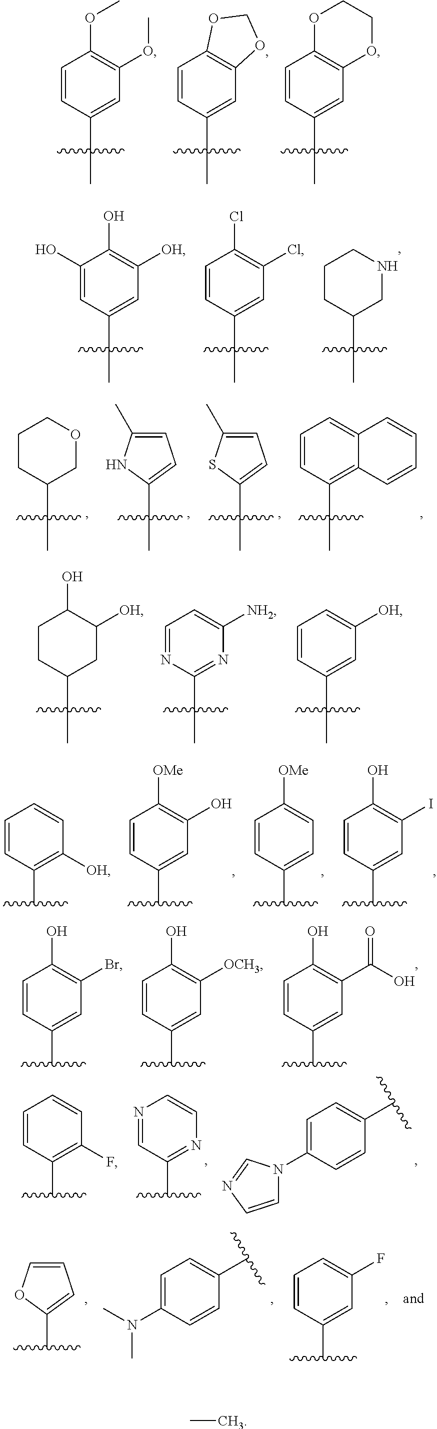

In certain embodiments, R² is

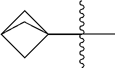 [SMILES: *C12CC(C1)C2]

and R¹ is selected from the group consisting of:

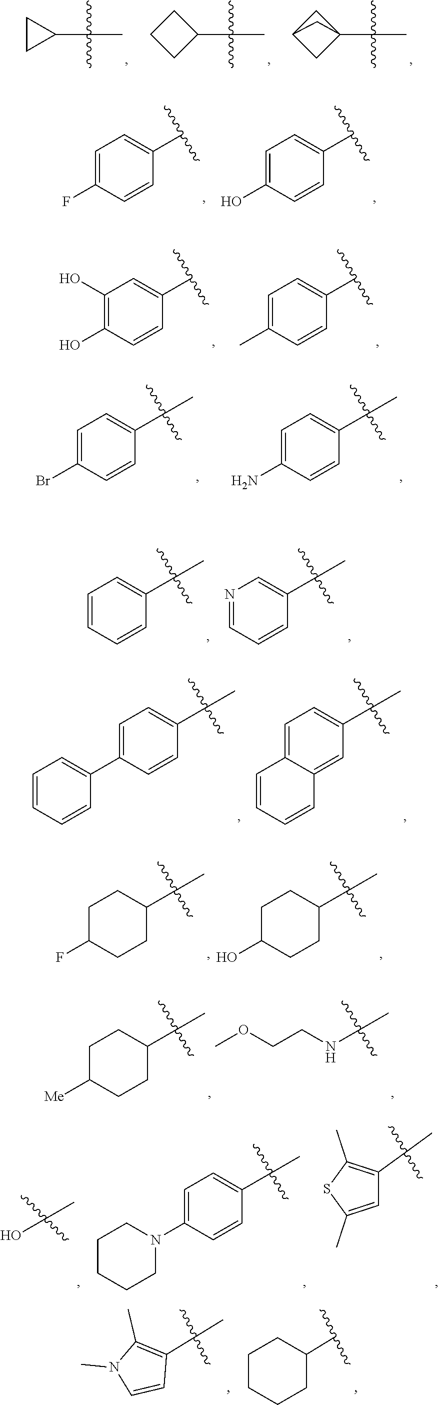

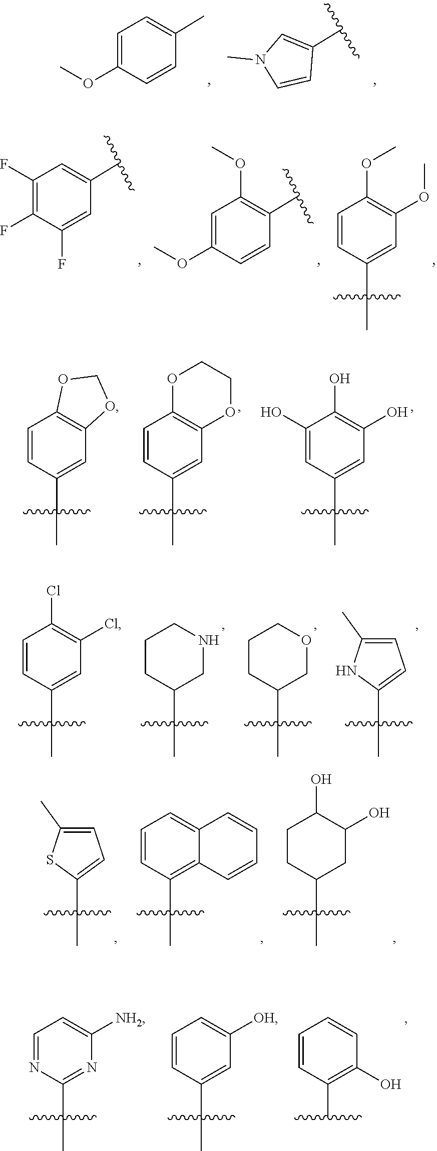

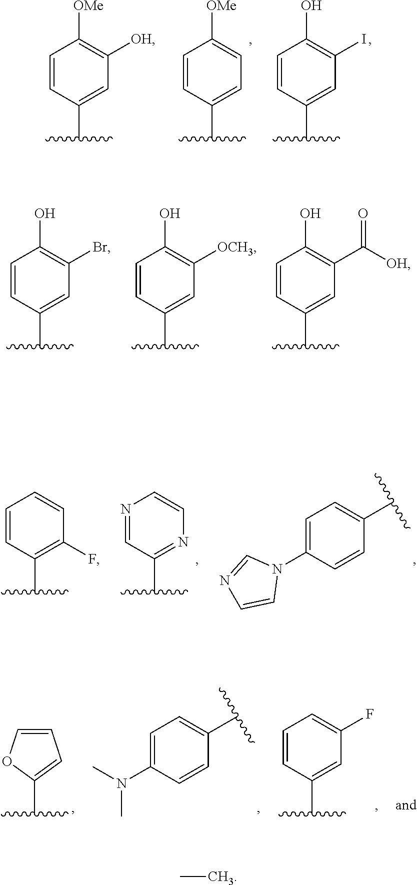

In certain embodiments, R¹

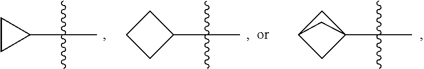

### and R² is

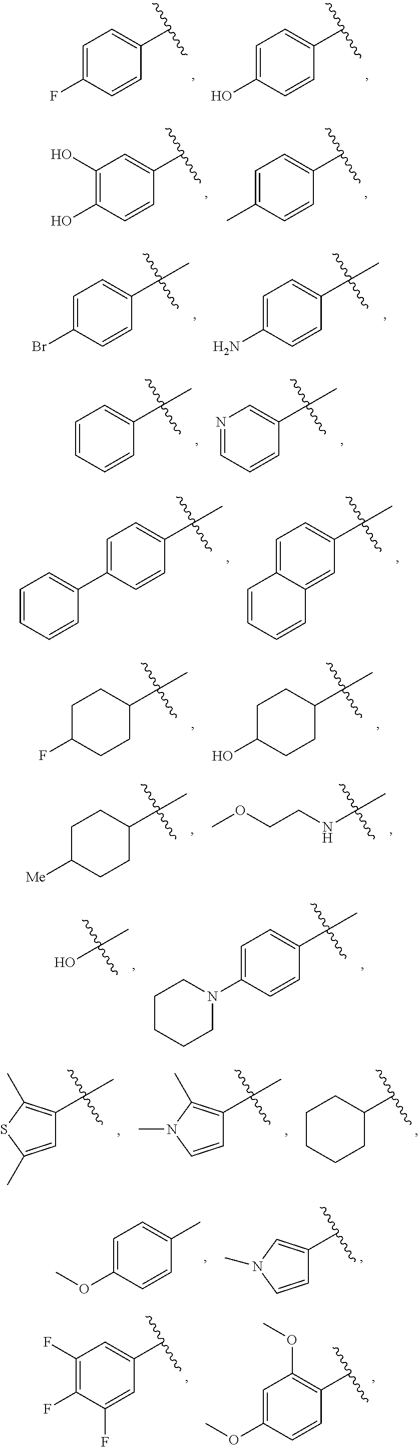

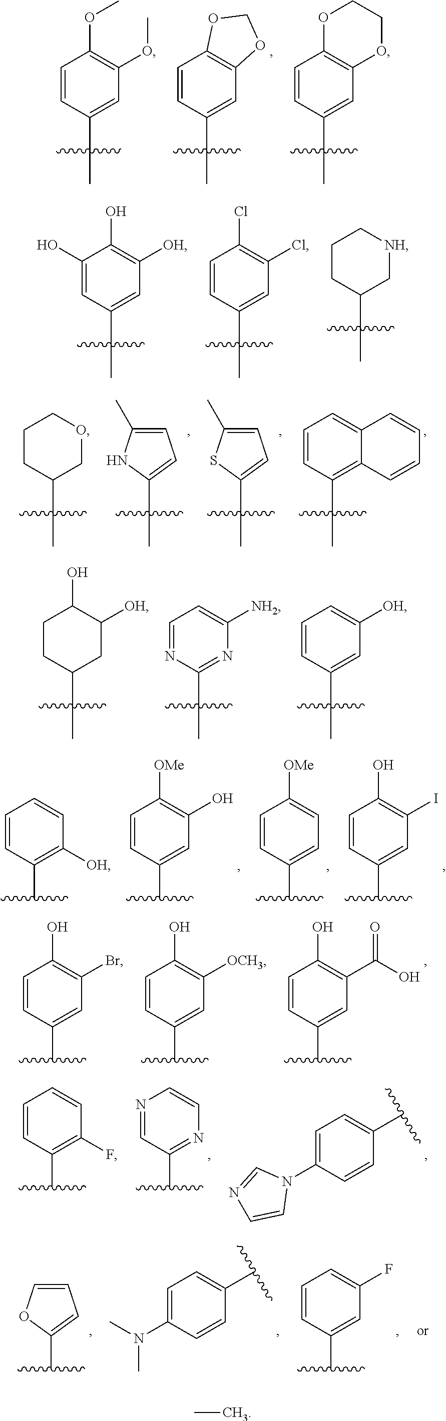

In certain embodiments, R²

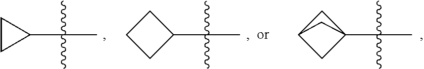

### is and R¹ is

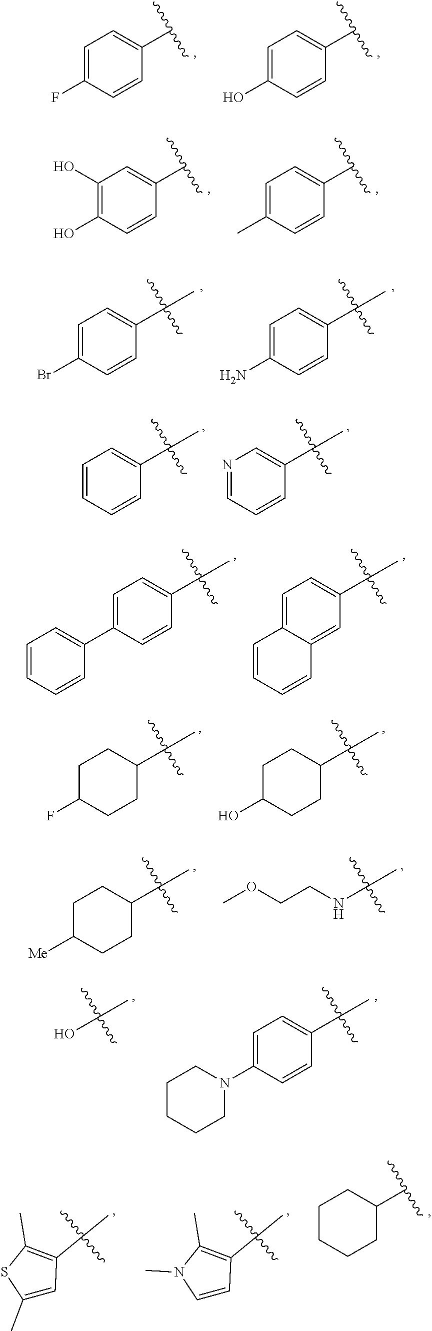

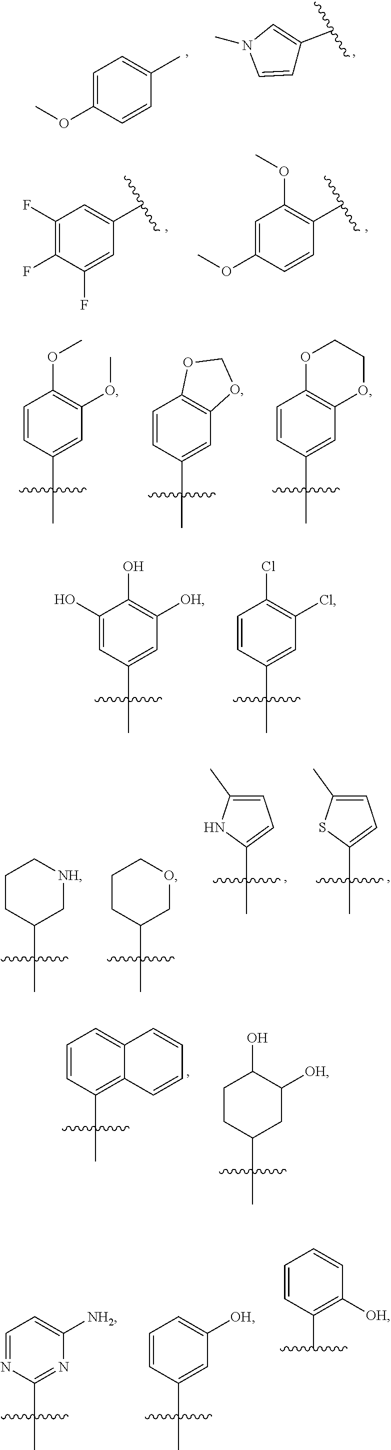

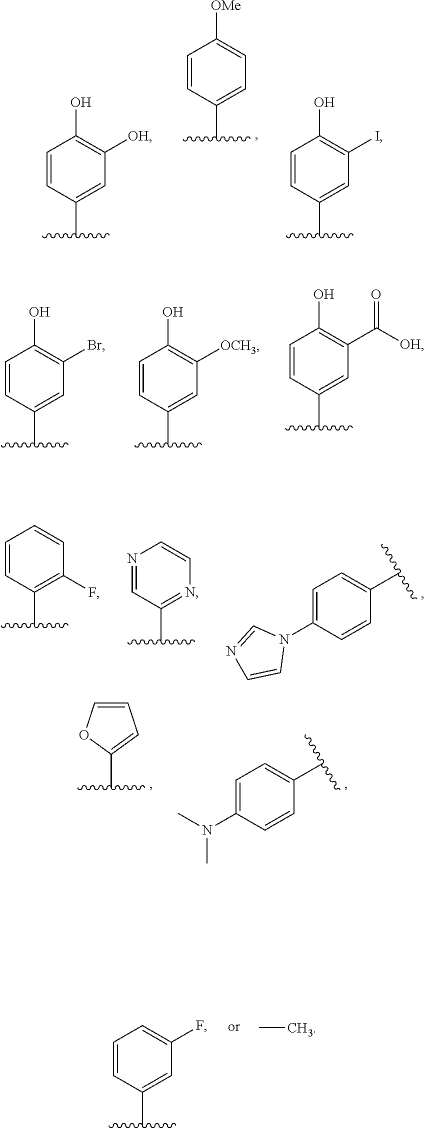

In some embodiments, each of R¹ and R² is independently selected from the group consisting of:

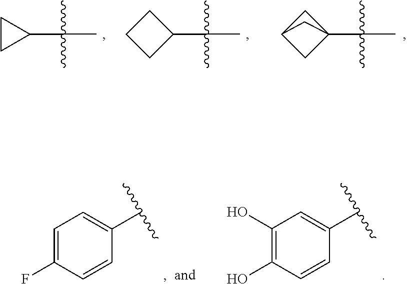

In certain embodiments. R¹ is

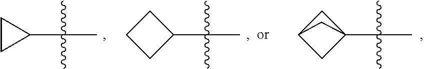

### and R² is

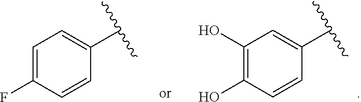 [SMILES: *c1ccc(F)cc1 ; *c1ccc(O)c(O)c1]

In some embodiments, R¹ is

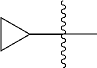 [SMILES: *C1CC1]

### and R² is

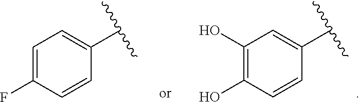 [SMILES: *c1ccc(F)cc1 ; *c1ccc(O)c(O)c1]

In some embodiments, R¹ is

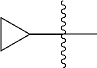 [SMILES: *C1CC1]

### and R² is

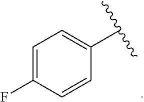 [SMILES: *c1ccc(F)cc1]

In some embodiments, R¹ is

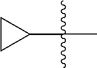 [SMILES: *C1CC1]

### and R² is

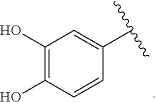 [SMILES: *c1ccc(O)c(O)c1]

In certain embodiments, R¹ is

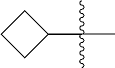 [SMILES: *C1CCC1]

### and R² is

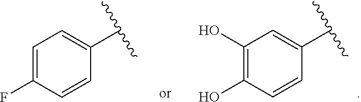 [SMILES: *c1ccc(F)cc1 ; *c1ccc(O)c(O)c1]

In certain embodiments, R¹ is

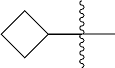 [SMILES: *C1CCC1]

### and R² is

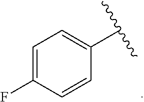 [SMILES: *c1ccc(F)cc1]

In certain embodiments, R¹ is

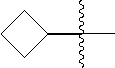 [SMILES: *C1CCC1]

### and R² is

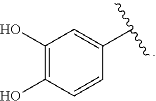 [SMILES: *c1ccc(O)c(O)c1]

In some embodiments, R¹ is

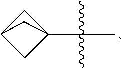 [SMILES: *C12CC(C1)C2]

### and R² is

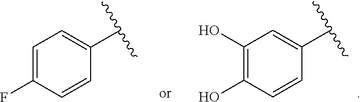 [SMILES: *c1ccc(F)cc1 ; *c1ccc(O)c(O)c1]

In some embodiments, R¹ is

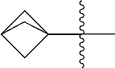 [SMILES: *C12CC(C1)C2]

### and R² is

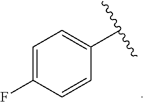 [SMILES: *c1ccc(F)cc1]

In some embodiments, R¹ is

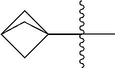 [SMILES: *C12CC(C1)C2]

### and R² is

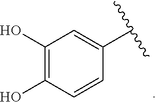 [SMILES: *c1ccc(O)c(O)c1]

In certain embodiments, R² is

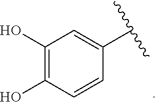 [SMILES: *c1ccc(O)c(O)c1]

In certain embodiments, R² is

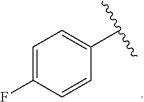 [SMILES: *c1ccc(F)cc1]

In some embodiments, R² is

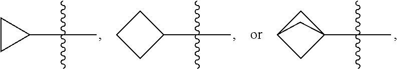

### and R¹ is

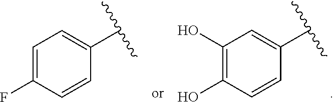 [SMILES: *c1ccc(F)cc1 ; *c1ccc(O)c(O)c1]

In certain embodiments, R² is

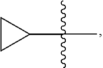 [SMILES: *C1CC1]

### and R¹ is

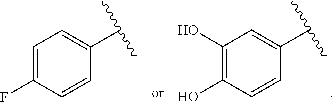 [SMILES: *c1ccc(F)cc1 ; *c1ccc(O)c(O)c1]

In certain embodiments. R² is

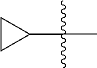 [SMILES: *C1CC1]

### and R¹ is

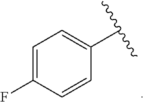 [SMILES: *c1ccc(F)cc1]

In certain embodiments, R² is

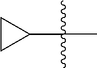 [SMILES: *C1CC1]

### and R¹ is

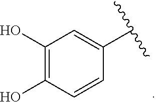 [SMILES: *c1ccc(O)c(O)c1]

In some embodiments. R² is

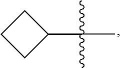 [SMILES: *C1CCC1]

### and R¹ is

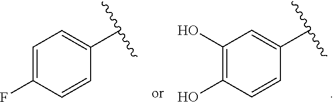 [SMILES: *c1ccc(F)cc1 ; *c1ccc(O)c(O)c1]

In some embodiments, R² is

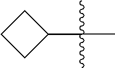 [SMILES: *C1CCC1]

### and R¹ is

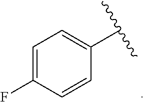 [SMILES: *c1ccc(F)cc1]

In some embodiments, R² is

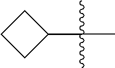 [SMILES: *C1CCC1]

### and R¹ is

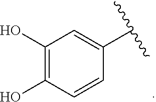 [SMILES: *c1ccc(O)c(O)c1]

In certain embodiments, R² is

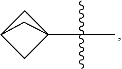 [SMILES: *C12CC(C1)C2]

### and R¹ is

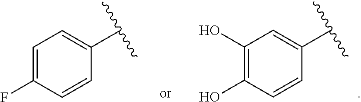 [SMILES: *c1ccc(F)cc1 ; *c1ccc(O)c(O)c1]

In certain embodiments, R² is

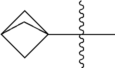 [SMILES: *C12CC(C1)C2]

### and R¹ is

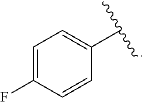 [SMILES: *c1ccc(F)cc1]

In certain embodiments, R² is

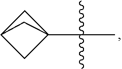 [SMILES: *C12CC(C1)C2]

### and R¹ is

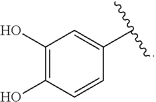 [SMILES: *c1ccc(O)c(O)c1]

In some embodiments, R¹ is

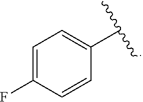 [SMILES: *c1ccc(F)cc1]

In some embodiments, R¹ is

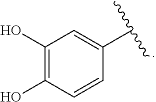 [SMILES: *c1ccc(O)c(O)c1]

In certain embodiments, the compound is of the formula:

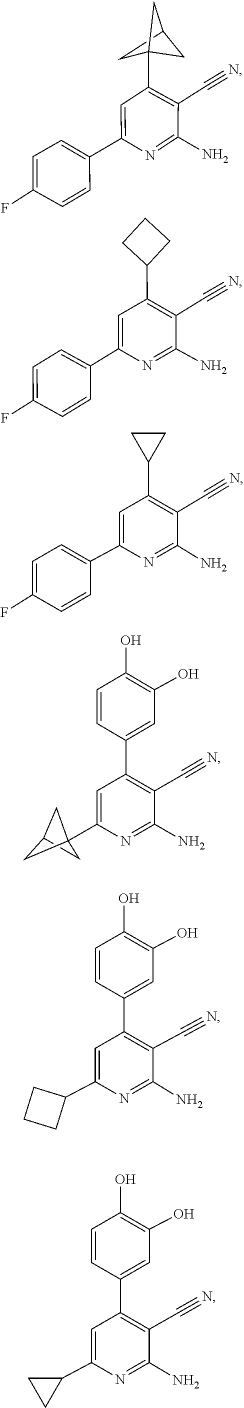 [SMILES: N#Cc1c(C23CC(C2)C3)cc(-c2ccc(F)cc2)nc1N ; N#Cc1c(C2CCC2)cc(-c2ccc(F)cc2)nc1N ; N#Cc1c(C2CC2)cc(-c2ccc(F)cc2)nc1N ; N#Cc1c(-c2ccc(O)c(O)c2)cc(C23CC(C2)C3)nc1N ; N#Cc1c(-c2ccc(O)c(O)c2)cc(C2CCC2)nc1N ; N#Cc1c(-c2ccc(O)c(O)c2)cc(C2CC2)nc1N]

or a pharmaceutically acceptable salt, solvate, hydrate, polymorph, co-crystal, tautomer, stereoisomer, isotopically labeled compound, or prodrug thereof. In certain embodiments, the compound is of the formula:

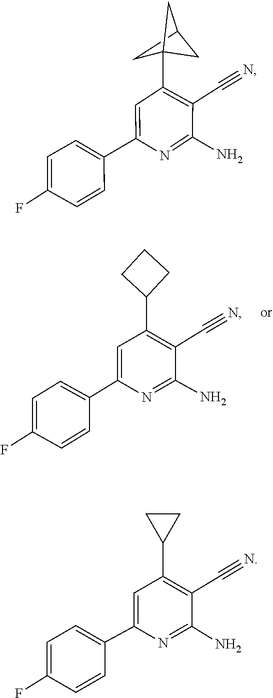 [SMILES: N#Cc1c(C23CC(C2)C3)cc(-c2ccc(F)cc2)nc1N ; N#Cc1c(C2CCC2)cc(-c2ccc(F)cc2)nc1N ; "or" ; N#Cc1c(C2CC2)cc(-c2ccc(F)cc2)nc1N]

In certain embodiments, the compound is of the formula

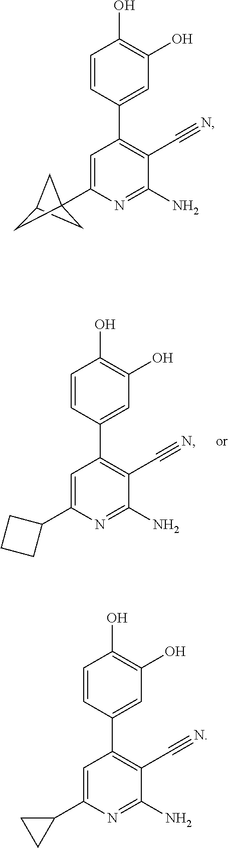 [SMILES: N#Cc1c(-c2ccc(O)c(O)c2)cc(C23CC(C2)C3)nc1N ; N#Cc1c(-c2ccc(O)c(O)c2)cc(C2CCC2)nc1N ; "or" ; N#Cc1c(-c2ccc(O)c(O)c2)cc(C2CC2)nc1N]

In certain embodiments, the compound is of the formula

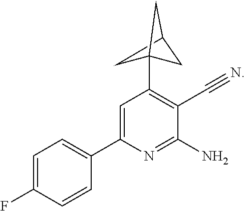 [SMILES: N#Cc1c(C23CC(C2)C3)cc(-c2ccc(F)cc2)nc1N]

In certain embodiments, the compound is of the formula

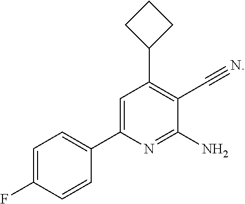 [SMILES: N#Cc1c(C2CCC2)cc(-c2ccc(F)cc2)nc1N]

In certain embodiments, the compound is of the formula

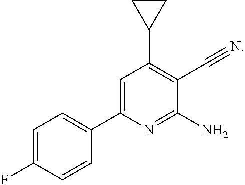 [SMILES: N#Cc1c(C2CC2)cc(-c2ccc(F)cc2)nc1N]

In certain embodiments, the compound is of the formula

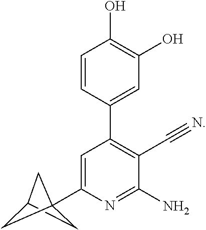 [SMILES: N#Cc1c(-c2ccc(O)c(O)c2)cc(C23CC(C2)C3)nc1N]

In certain embodiments, the compound is of the formula

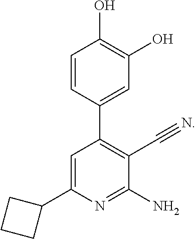 [SMILES: N#Cc1c(-c2ccc(O)c(O)c2)cc(C2CCC2)nc1N]

In certain embodiments, the compound is of the formula

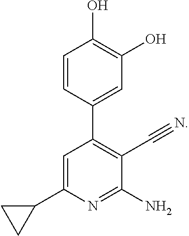 [SMILES: N#Cc1c(-c2ccc(O)c(O)c2)cc(C2CC2)nc1N]

In some embodiments, the compound is selected from the group consisting of: 2-amino-4-(3,4-dihydroxyphenyl)-6-(3-fluorophenyl)nicotinonitrile; 2-amino-4-(3,4-dihydroxyphenyl)-6-(2-fluorophenyl)nicotinonitrile; 6-(4-bromophenyl)-4-(3,4-dihydroxyphenyl)-2-imino-2,3-dihydropyridine-3-carbonitrile; 4-(3,4-dihydroxyphenyl)-6-(4-hydroxyphenyl)-2-imino-1,2-dihydropyridine-3-carbonitrile; 4-(3,4-dihydroxyphenyl)-2-imino-6-(p-tolyl)-1,2-dihydropyridine-3-carbonitrile; 4-(3,4-dihydroxyphenyl)-2-imino-6-(4-methoxyphenyl)-2,3-dihydropyridine-3-carbonitrile; 4-(3,4-dihydroxyphenyl)-6-imino-1,6-dihydro-[2,3′-bipyridine]-5-carbonitrile, 2-amino-4-(3,4-dihydroxyphenyl)-6-(furan-2-yl)nicotinonitrile; 4-(3,4-dihydroxyphenyl)-2-imino-6-(pyrazin-2-yl)-2,3-dihydropyridine-3-carbonitrile; 6-([1,1′-biphenyl]-4-yl)-4-(3,4-dihydroxyphenyl)-2-imino-1,2-dihydropyridine-3-carbonitrile; 4-(3,4-dihydroxyphenyl)-2-imino-6-(naphthalen-2-yl)-1,2-dihydropyridine-3-carbonitrile; 2-amino-6-cyclohexyl-4-(3,4-dihydroxyphenyl)nicotinonitrile; 2-amino-4-(3,4-dihydroxyphenyl)-6-methylnicotinonitrile; 6-(4-aminophenyl)-4-(3,4-dihydroxyphenyl)-2-imino-1,2-dihydropyridine-3-carbonitrile; 4-(3,4-dihydroxyphenyl)-2-imino-6-phenyl-1,2-dihydropyridine-3-carbonitrile; 6-(4-(1H-imidazol-1-yl)phenyl)-2-amino-4-(3,4-dihydroxyphenyl)nicotinonitrile; 2-amino-4-(3,4-dihydroxyphenyl)-6-(4-(dimethylamino)phenyl)nicotinonitrile; 4-(3,4-dihydroxyphenyl)-6-(4-fluorophenyl)-2-oxo-1,2-dihydropyridine-3-carbonitrile; 4-(3,4-dihydroxyphenyl)-6-(4-fluorophenyl)-2-imino-5-methyl-1,2-dihydropyridine-3-carbonitrile; 6-([1,1′-biphenyl]-4-yl)-4-(3,4-dihydroxyphenyl)-2-oxo-2,3-dihydropyridine-3-carbonitrile; 4-(3,4-dihydroxyphenyl)-6-(4-hydroxyphenyl)-2-oxo-2,3-dihydropyridine-3-carbonitrile; 5-(2-amino-6-(4-aminophenyl)-3-cyanopyridin-4-yl)-2-hydroxybenzoic acid; 4-(3,4-dichlorophenyl)-6-(4-fluorophenyl)-2-oxo-1,2-dihydropyridine-3-carbonitrile; 6-(4-fluorophenyl)-2-oxo-4-phenyl-1,2-dihydropyridine-3-carbonitrile; 6-(4-fluorophenyl)-2-imino-4-(tetrahydro-2H-pyran-3-yl)-1,2-dihydropyridine-3-carbonitrile; 6-(4-fluorophenyl)-4-(5-methylfuran-2-yl)-2-oxo-1,2-dihydropyridine-3-carbonitrile; 6-(4-fluorophenyl)-2-imino-4-(naphthalen-2-yl)-1,2-dihydropyridine-3-carbonitrile; 6-(4-fluorophenyl)-2-hydroxy-4-(2-hydroxyphenyl)nicotinonitrile; 6-(4-fluorophenyl)-4-(3-hydroxyphenyl)-2-oxo-1,2-dihydropyridine-3-carbonitrile; 6-(4-fluorophenyl)-4-(4-hydroxyphenyl)-2-oxo-1,2-dihydropyridine-3-carbonitrile; 6-(4-fluorophenyl)-4-(4-hydroxy-3-methoxyphenyl)-2-oxo-1,2-dihydropyridine-3-carbonitrile; 6-(4-fluorophenyl)-2-imino-4-phenyl-1,2-dihydropyridine-3-carbonitrile; 6-(4-fluorophenyl)-2-imino-4-(5-methylfuran-2-yl)-1,2-dihydropyridine-3-carbonitrile; 6-(4-fluorophenyl)-2-imino-4-(5-methylthiophen-2-yl)-1,2-dihydropyridine-3-carbonitrile; 6-(4-fluorophenyl)-4-(4-hydroxyphenyl)-2-oxo-1,2-dihydropyridine-3-carbonitrile; 2-amino-6-(4-fluorophenyl)-4-(2-hydroxyphenyl)nicotinonitrile; 6-(4-fluorophenyl)-4-(4-hydroxy-3-methoxyphenyl)-2-imino-1,2-dihydropyridine-3-carbonitrile; 6-(4-fluorophenyl)-4-(3-hydroxy-4-methoxyphenyl)-2-imino-1,2-dihydropyridine-3-carbonitrile; 6′-(4-fluorophenyl)-2′-imino-1′,2′-dihydro-[3,4′-bipyridine]-3′-carbonitrile; 5-(3-cyano-6-(4-fluorophenyl)-2-imino-1,2-dihydropyridin-4-yl)-2-hydroxybenzoic acid; 4-(3-bromo-4-hydroxyphenyl)-6-(4-fluorophenyl)-2-imino-1,2-dihydropyridine-3-carbonitrile; 6-(4-fluorophenyl)-4-(4-hydroxy-3-iodophenyl)-2-imino-1,2-dihydropyridine-3-carbonitrile; 6-(bicyclo[1.1.1]pentan-1-yl)-4-(3,4-dihydroxyphenyl)-2-imino-2,3-dihydropyridine-3-carbonitrile; 2-amino-6-cyclopropyl-4-(3,4-dihydroxyphenyl) nicotinonitrile; and 2-amino-6-cyclobutyl-4-(3,4-dihydroxyphenyl) nicotinonitrile.

In some embodiments, the compound is

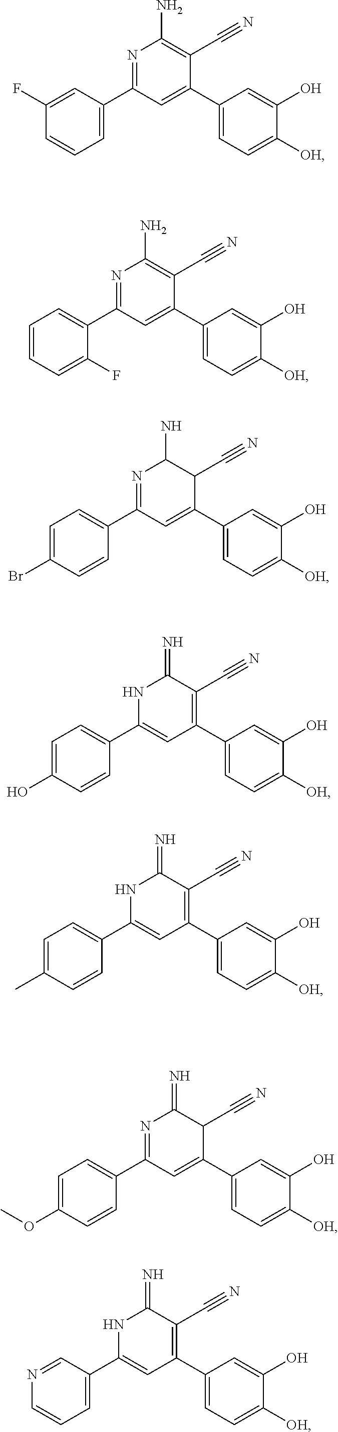

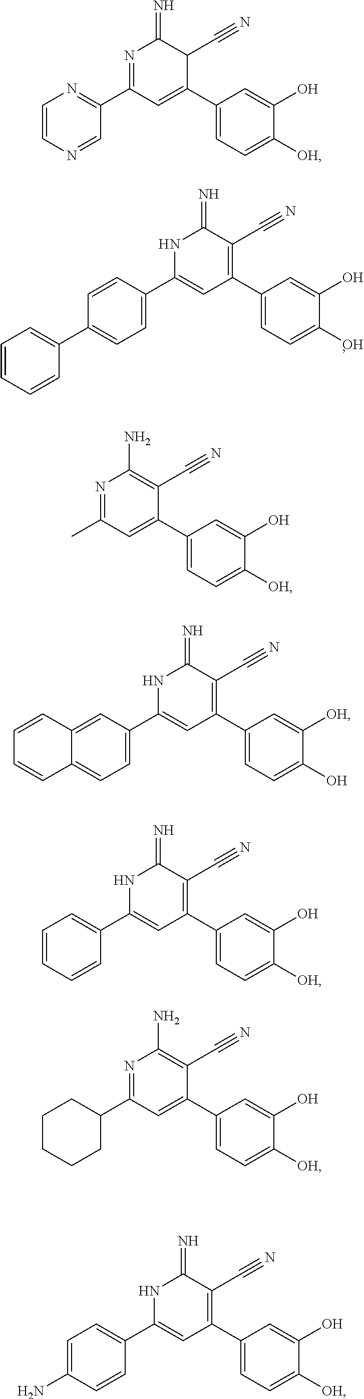

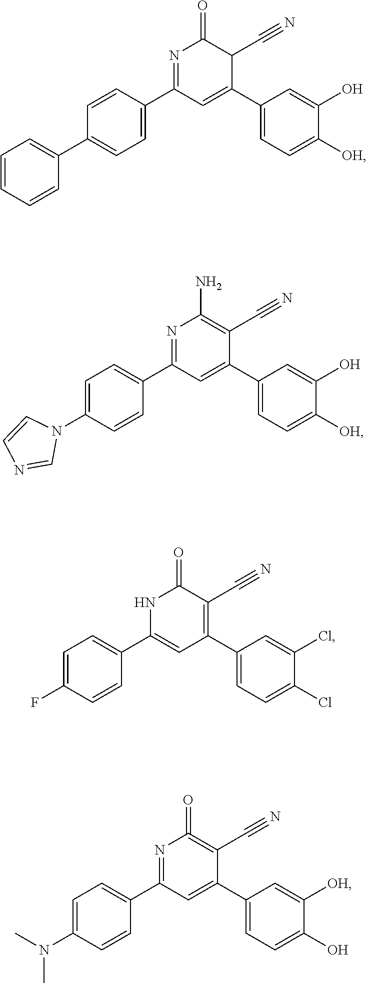

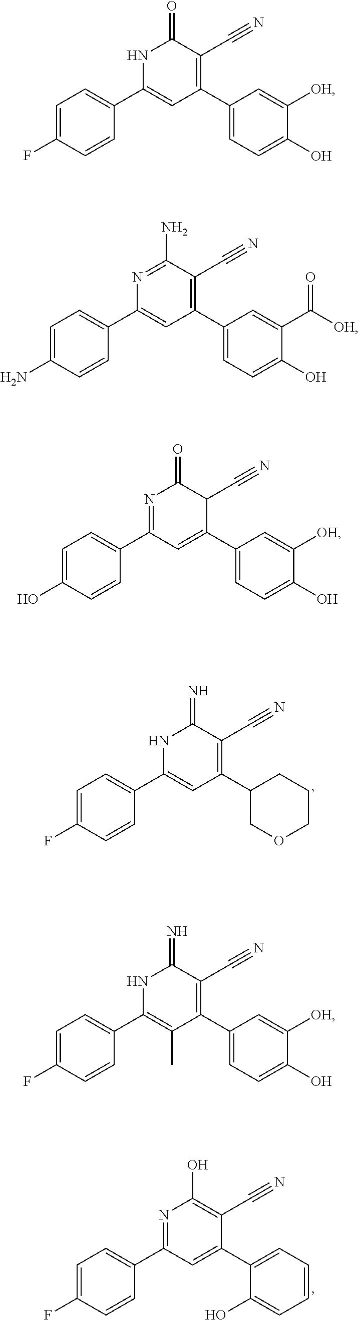 [SMILES: N#Cc1c(-c2ccc(O)c(O)c2)cc(-c2ccc(F)cc2)[nH]c1=O ; N#Cc1c(-c2ccc(O)c(C(=O)O)c2)cc(-c2ccc(N)cc2)nc1N ; N#CC1C(=O)N=C(c2ccc(O)cc2)C=C1c1ccc(O)c(O)c1 ; N#Cc1c(C2CCCOC2)cc(-c2ccc(F)cc2)[nH]c1=N ; Cc1c(-c2ccc(F)cc2)[nH]c(=N)c(C#N)c1-c1ccc(O)c(O)c1 ; N#Cc1c(-c2ccccc2O)cc(-c2ccc(F)cc2)nc1O]

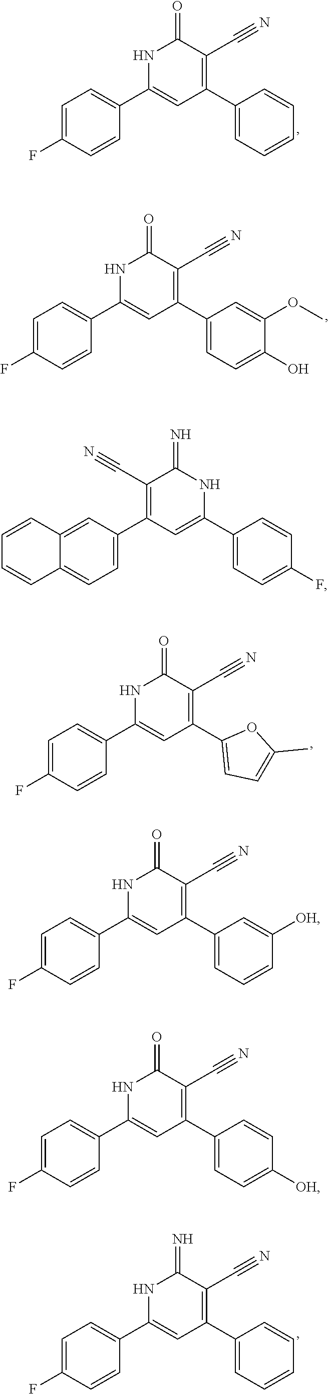

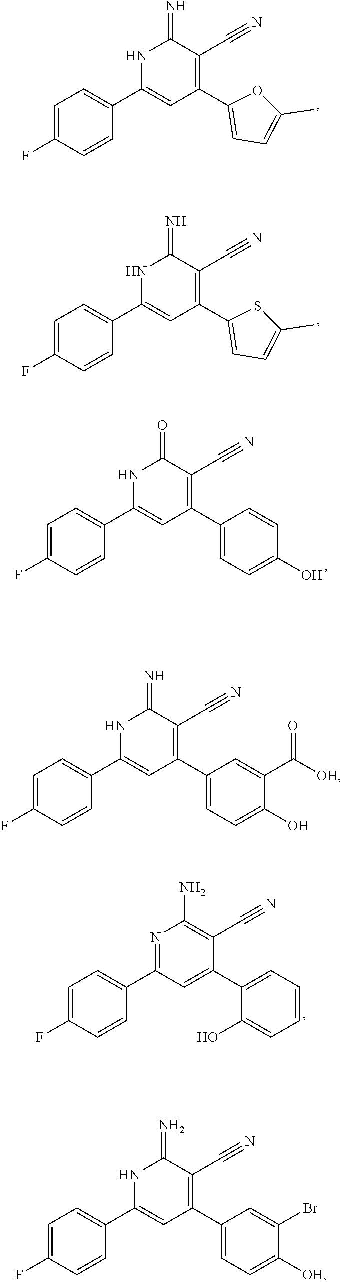 [SMILES: Cc1ccc(-c2cc(-c3ccc(F)cc3)[nH]c(=N)c2C#N)o1 ; Cc1ccc(-c2cc(-c3ccc(F)cc3)[nH]c(=N)c2C#N)s1 ; N#Cc1c(-c2ccc(O)cc2)cc(-c2ccc(F)cc2)[nH]c1=O ; N#Cc1c(-c2ccc(O)c(C(=O)O)c2)cc(-c2ccc(F)cc2)[nH]c1=N ; N#Cc1c(-c2ccccc2O)cc(-c2ccc(F)cc2)nc1N ; N#Cc1c(-c2ccc(O)c(Br)c2)cc(-c2ccc(F)cc2)[nH]c1=N]

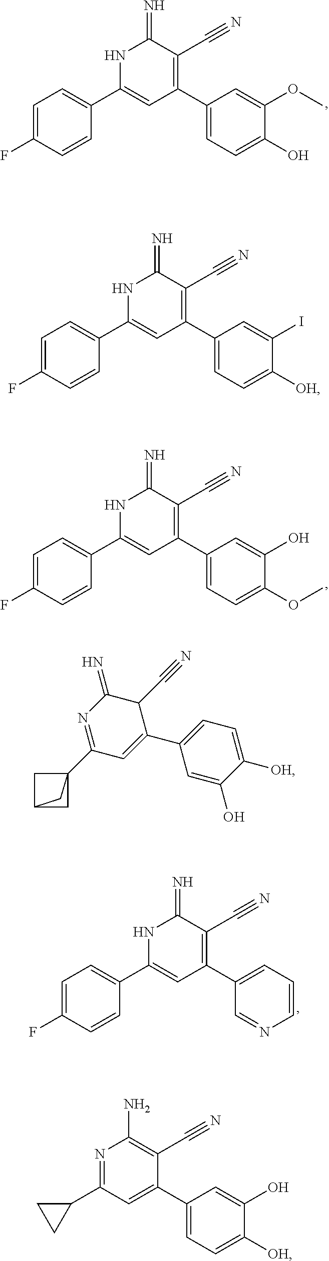 [SMILES: COc1cc(-c2cc(-c3ccc(F)cc3)[nH]c(=N)c2C#N)ccc1O ; N#Cc1c(-c2ccc(O)c(I)c2)cc(-c2ccc(F)cc2)[nH]c1=N ; COc1ccc(-c2cc(-c3ccc(F)cc3)[nH]c(=N)c2C#N)cc1O ; N#CC1C(=N)N=C(C23CC(C2)C3)C=C1c1ccc(O)c(O)c1 ; N#Cc1c(-c2cccnc2)cc(-c2ccc(F)cc2)[nH]c1=N ; N#Cc1c(-c2ccc(O)c(O)c2)cc(C2CC2)nc1N]

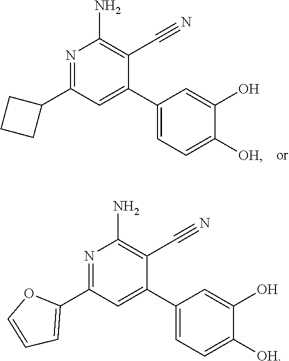 [SMILES: N#Cc1c(-c2ccc(O)c(O)c2)cc(C2CCC2)nc1N ; N#Cc1c(-c2ccc(O)c(O)c2)cc(-c2ccco2)nc1N]

In some embodiments, the compound is of the formula shown in Table 1.

### Pharmaceutical Compositions, Kits, and Administration

In another aspect, the present disclosure provides a pharmaceutical composition comprising a compound of the present disclosure, or a pharmaceutically acceptable salt, solvate, hydrate, polymorph, co-crystal, tautomer, stereoisomer, isotopically labeled compound, or prodrug thereof, and a pharmaceutically acceptable carrier.

In some embodiments, the pharmaceutical composition further comprises an additional pharmaceutical agent.

In one aspect of the present disclosure, provided is a therapeutic composition comprising the compound described herein or the pharmaceutical composition described herein, and an at least one second therapeutic molecule.

In some embodiments, the compound and the at least one second therapeutic molecule are conjugated or ligated to one another.

In some embodiments, the compound and the at least one second therapeutic molecule are both present in or on a scaffold material or molecule. In some embodiments, the scaffold is a polymer. In some embodiments the scaffold is a polymer comprising polyethylene glycol, alginate, dextran, chitosan, or poly(lactic-co-glycolyl acid) (PLGA).

In some embodiments, the compound and the at least second therapeutic molecule are bound, conjugated, or ligated to each other or to a scaffold. In some embodiments, binding is non-covalent, e.g., by hydrogen, electrostatic, or van der Waals interactions. In some embodiments, binding is covalent. The term “conjugated” refers to the attachment of at least two entities to form one entity. In some embodiments, the joining of the two entities is direct (e.g., via covalent or non-covalent bonds) or indirect (e.g., via linkers etc.). In some embodiments, conjugation is by means of linkers, chemical modification, peptide linkers, chemical linkers, covalent or noncovalent bonds, or protein fusion or by any means known to one skilled in the art, e.g., click chemistry or biorthogonal chemistries as described in Devaraj. ACS Central Science 2018 4:952-9; which is incorporated by reference herein in its entirety. In some embodiments, the joining or conjugation is permanent or reversible.

In some embodiments, the compound and optionally the at least one second therapeutic molecule are present in or on a scaffold material or molecule. In some embodiments where both the compound and the at least one second therapeutic molecule are present, they are present in or on the same scaffold. In some embodiments, the scaffold is a lipid bilayer or solid surface. In some embodiments, the lipid bilayer surface is a liposome. In some embodiments, the surface is a solid surface or solid support. In some embodiments, the scaffold is beads (such as magnetic beads, polystyrene beads, or gold beads); resin; fiber; sheet; biocompatible polymer or material; a nanoparticle; a matrix; a hydrogel; and a biomaterial, biocompatible, and/or biodegradable scaffold material; or the like.

In some embodiments, the scaffold further comprises a nanocarrier. For example, various nanocarriers for targeting to, e.g., cancer tumors (e.g., via the enhanced permeability and retention effect) are known in the art and can include but are not limited to PLGA nanoparticles, poly(carboxyphenoxypropane/sebacic acid), poly(glycerol monostearate-co-caprolactone), and the like. Such nanocarriers and their use are described in the art, e.g., Rosenblum et al. Nature Communications 2018 9:1410; which is incorporated by reference herein in its entirety.

In one aspect of the present disclosure, provided is a kit comprising: a compound described herein or a pharmaceutically acceptable salt, solvate, hydrate, polymorph, co-crystal, tautomer, stereoisomer, isotopically labeled compound, or prodrug thereof, or the pharmaceutical composition described herein, or the therapeutic composition described herein; and instructions for using the compound, or a pharmaceutically acceptable salt, solvate, hydrate, polymorph, co-crystal, tautomer, stereoisomer, isotopically labeled compound, or prodrug thereof, or the pharmaceutical composition or the therapeutic composition.

In certain embodiments, the compound described herein is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a proliferative disease in a subject in need thereof. In certain embodiments, the effective amount is an amount effective for preventing a proliferative disease in a subject in need thereof. In certain embodiments, the effective amount is an amount effective for preventing a painful condition in a subject in need thereof. In certain embodiments, the effective amount is an amount effective for treating an inflammatory disorder in a subject in need thereof. In certain embodiments, the effective amount is an amount effective for treating an immune disorder in a subject in need thereof. In certain embodiments, the effective amount is an amount effective for reducing the risk of developing a disease (e.g., proliferative disease, immune disease, inflammatory disease, or painful condition) in a subject in need thereof. In certain embodiments, the effective amount is an amount effective for inhibiting the activity of cytokine in a subject or cell. In some embodiments, the activity is aberrant activity.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the cell is present in vitro. In certain embodiments, the cell is present ex vivo.

In certain embodiments, the effective amount is an amount effective for inhibiting the activity of a cytokine by at least about 10%, at least about 20%, at least about 30%, at least about 40%, at least about 50%, at least about 60%, at least about 70%, at least about 80%, at least about 90%, at least about 95%, or at least about 98%. In certain embodiments, the effective amount is an amount effective for inhibiting the activity of the cytokine by not more than 10%, not more than 20%, not more than 30%, not more than 40%, not more than 50%, not more than 60%, not more than 70%, not more than 80%, not more than 90%, not more than 95%, or not more than 98%. In certain embodiments, the effective amount is an amount effective for inhibiting the activity of by a range between a percentage described in this paragraph and another percentage described in this paragraph, inclusive.

Pharmaceutical compositions described herein can be prepared by any method known in the art of pharmaceutics. In general, such preparatory methods include bringing the compound described herein (i.e., the “active ingredient”) into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents or fillers, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents or fillers include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, starches (such as dry starch, cornstarch), sugars (such as powdered sugar), calcium trisulfate, carboxymethylcellulose calcium, dextrate, dextrin, dextrose, fructose, lactitol, lactose, magnesium carbonate, magnesium, maltitol, maltodextrin, maltose, sucrose, glucose, mannitol, silicic acid, xylitol, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary disintegrating agents or disintegrants include agar, algin, alginic acid, sodium alginate, silicates, sodium carbonate, calcium carbonate, carboxymethylcellulose, cellulose, clay, colloidal silicon dioxide, croscarmellose sodium, crospovidone, rubber, magnesium silicate, methylcellulose, potassium krillin, hydroxypropylcellulose (e.g., low substituted Hydroxypropylcellulose), crosslinked poly vinylpyrrolidone, hydroxypropylcellulose, and starch (e.g., sodium glycolate starch, potato or tapioca starch).

Exemplary binding agents include starch (e.g., glycolate starch, cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof.

Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate. D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include agar, ethyl oleate, ethyl laurate, glycerin, blyceryl palmitostearate, magnesium oxide, magnesium stearate, mannitol, poloxamer, glycol, sodium stearyl, sorbitol, zinc stearate, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

In some embodiments, injectable preparations of the compositions disclosed herein are in the form of a ready-to-use (“RTU”) preparation that can be directly administered to a subject. In some embodiments, the RTU preparation is a suspension. In some embodiments, the RTU preparation is a solution. In some embodiments, the RTU preparation is an emulsion. In some embodiments, injectable preparations of the compositions disclosed herein are in the form of a solid that is reconstituted prior to administration. In some embodiments, the solid is a lyophilized solid. In some embodiments, injectable preparations of the compositions disclosed herein are in the form of a liquid or suspension that is diluted prior to administration.

In some embodiments, the pharmaceutical compositions disclosed herein comprise a bulking agent. Bulking agents can be used, e.g., to improve the appearance of a solid composition, to provide visible “bulk” to demonstrate product quality or to facilitate preparation, e.g., of a solid composition prepared for reconstitution prior to administration. Bulking agents can be used for low dose (high potency) drugs that do not have the necessary bulk to support their own structure or provide a visible composition in a unit dosage form. Bulking agents are used in lyophilized formulations. Bulking agents provide a desirable structure for a lyophilized cake comprising pores that provide the means for vapor to escape from the product during lyophilization cycles, and facilitate dissolution on reconstitution. In some embodiments, the bulking agent is mannitol, lactose, sucrose, dextran, trehalose, povidone, dextran, glycine, isoleucine, methionine, or a cyclodextrin (e.g., (2-hydroxypropyl)-β-cyclodextrin).

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinyl pyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin. (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polyethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

A pharmaceutical composition described herein can be prepared, packaged, and/or sold in a formulation for ophthalmic administration. Such formulations may, for example, be in the form of eye drops including, for example, a 0.1-1.0% (w/w) solution and/or suspension of the active ingredient in an aqueous or oily liquid carrier or excipient. Such drops may further comprise buffering agents, salts, and/or one or more other of the additional ingredients described herein. Other ophthalmically-administrable formulations which are useful include those which comprise the active ingredient in microcrystalline form and/or in a liposomal preparation. Ear drops and/or eye drops are also contemplated as being within the scope of this disclosure.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, intradermal, rectal, intravaginal, intraperitoneal, topical (as by powders, ointments, creams, and/or drops), mucosal, nasal, buccal, sublingual; by intratracheal instillation, bronchial instillation, and/or inhalation, and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a compound described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a cytokine in a subject or cell), improve bioavailability, improve safety, reduce drug resistance, reduce and/or modify metabolism, inhibit excretion, and/or modify, distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the compound or composition described herein in a single dose or composition or administered separately in different doses or compositions. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, anti-proliferative agents, anti-cancer agents, anti-angiogenesis agents, steroidal or non-steroidal anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, anesthetics, anti-coagulants, inhibitors of an enzyme, steroidal agents, steroidal or antihistamine, antigens, vaccines, antibodies, decongestant, sedatives, opioids, analgesics, anti-pyretics, hormones, and prostaglandins. In certain embodiments, the additional pharmaceutical agent is an anti-proliferative agent. In certain embodiments, the additional pharmaceutical agent is an anti-cancer agent. In certain embodiments, the additional pharmaceutical agent is an anti-viral agent. In certain embodiments, the additional pharmaceutical agent is an binder or inhibitor of a protein kinase. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the compounds described herein or pharmaceutical compositions can be administered in combination with an anti-cancer therapy including, but not limited to, surgery, radiation therapy, transplantation (e.g., stem cell transplantation, bone marrow transplantation), immunotherapy, and chemotherapy. Additional pharmaceutical agents include small organic molecules such as drug compounds (e.g., compounds approved by the US Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids. DNAs. RNAs, nucleotides, nucleosides, oligonucleotides, antisense oligonucleotides, lipids, hormones, vitamins and cells.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease, inflammatory disease, immune disease, proliferative disease) in a subject in need thereof. In certain embodiments, the kits are useful for preventing a disease (e.g., proliferative disease, hematological disease, neurological disease, painful condition, psychiatric disorder, or metabolic disorder) in a subject in need thereof. In certain embodiments, the kits are useful for reducing the risk of developing a disease (e.g., proliferative disease, hematological disease, neurological disease, painful condition, psychiatric disorder, or metabolic disorder) in a subject in need thereof. In certain embodiments, the kits are useful for inhibiting the activity (e.g., aberrant activity, such as increased activity) of a cytokine in a subject or cell.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease, hematological disease, neurological disease, painful condition, psychiatric disorder, or metabolic disorder) in a subject in need thereof. In certain embodiments, the kits and instructions provide for preventing a disease (e.g., proliferative disease, hematological disease, neurological disease, painful condition, psychiatric disorder, or metabolic disorder) in a subject in need thereof. In certain embodiments, the kits and instructions provide for reducing the risk of developing a disease (e.g., proliferative disease, hematological disease, neurological disease, painful condition, psychiatric disorder, or metabolic disorder) in a subject in need thereof. In certain embodiments, the kits and instructions provide for inhibiting the activity (e.g., aberrant activity, such as increased activity) of a cytokine in a subject or cell. A kit described herein may include one or more additional pharmaceutical agents described herein as a separate composition.

### Methods of Treatment and Uses

In another aspect, provided herein is use of the compound described herein, or a pharmaceutically acceptable salt, solvate, hydrate, polymorph, co-crystal, tautomer, stereoisomer, isotopically labeled compound, or prodrug thereof, or the pharmaceutical composition described herein, or the therapeutic composition described herein, in the manufacture of a medicament for treating a disease.

In one aspect of the present disclosure, provided is a method of treating a disease in a subject in need thereof, the method comprising administering to the subject in need thereof a therapeutically effective amount of a compound described herein, or a pharmaceutically acceptable salt, solvate, hydrate, polymorph, co-crystal, tautomer, stereoisomer, isotopically labeled derivative, or prodrug thereof, or the pharmaceutical composition described herein, or the therapeutic composition described herein.

In some embodiments, the disease is a proliferative disease, immune disease, autoimmune disorder, inflammatory disorder, allergies, infection, or fibrosis.

In some embodiments, the disease is selected from the group consisting of asthma, rheumatoid arthritis, allergies, cancer, atopic dermatitis, eosinophilic esophagitis, prurigo nodularis, chronic rhinosinusitis with nasal polyposis, or chronic obstructive pulmonary disease (COPD), tissue/organ fibrosis, implant fibrosis, systemic lupus erythematosus, bullous pemphigoid, chronic spontaneous urticaria. Graves' disease, multiple sclerosis, pemphigus, or uveitis.

In certain embodiments, the disease is a disease associated with overexpression and/or aberrant activity of IL-4. In some embodiments, the method further comprises inhibiting IL-4. In some embodiments, the method comprises contacting IL-4 with a compound of pharmaceutical composition described herein. In some embodiments, the compound inhibits IL-4. In some embodiments, the compound binds IL-4 but does not inhibit IL-4.

In certain embodiments, the disease is a proliferative disease. In some embodiments, the proliferative disease is cancer. In certain embodiments, the cancer is carcinoma, sarcoma, lymphoma, or germinoma. In some embodiments, the cancer is not leukemia.

In certain embodiments, the disease is an immune disorder. In some embodiments, the immune disorder is asthma, rheumatoid arthritis, allergies, atopic dermatitis, eosinophilic esophagitis, prurigo nodularis, chronic rhinosinusitis with nasal polyposis, chronic obstructive pulmonary disease (COPD), tissue/organ fibrosis, implant fibrosis, systemic lupus erythematosus, bullous pemphigoid, chronic spontaneous urticaria, Graves' disease, multiple sclerosis, pemphigus, or uveitis.
some embodiments, the disease is an inflammatory disorder or autoimmune disorder.

In some embodiments, the disease is an inflammatory disorder. In some embodiments, the inflammatory disorder is asthma, allergies, atopic dermatitis, eosinophilic esophagitis, prurigo nodularis, chronic rhinosinusitis with nasal polyposis, chronic obstructive pulmonary disease (COPD), tissue/organ fibrosis, implant fibrosis, or uveitis.

In some embodiments, the disease is an autoimmune disease. In certain embodiments, the autoimmune disease is rheumatoid arthritis, systemic lupus erythematosus, bullous pemphigoid, chronic spontaneous urticaria. Graves' disease, multiple sclerosis, or pemphigus.

In another aspect, provided herein is a method of reducing an inflammatory response in a subject in need thereof, the method comprising administering to the subject a composition described herein or a pharmaceutically acceptable salt, solvate, hydrate, polymorph, co-crystal, tautomer, stereoisomer, isotopically labeled compound, or prodrug thereof, or the pharmaceutical composition described herein, or the therapeutic composition described herein.

In one aspect, described herein is a method of inhibiting the activity of a cytokine in a subject in need thereof, the method comprising administering to the subject an effective amount of a compound described herein or a pharmaceutically acceptable salt, solvate, hydrate, polymorph, co-crystal, tautomer, stereoisomer, isotopically labeled compound, or prodrug thereof, or the pharmaceutical composition described herein or the therapeutic composition described herein.

In certain embodiments, the cytokine is IL-4. In some embodiments, inhibition is selective for IL-4 over IL-13. In certain embodiments, the method further comprises inhibition of IL-4 induced STAT-6 phosphorylation. In some embodiments, the method further comprises administering to the subject in need thereof an additional therapy.

In some embodiments, the additional therapy is a cytotoxic chemotherapy, epigenetic modifier, glucocorticoid, immunotherapy, cell-based therapies, nucleic acid therapies, vaccines, radiotherapy, protein degraders, antibody-drug conjugates, or gene therapy.

In some embodiments the subject is a human. In certain embodiments, the subject is a non-human mammal.

In another aspect, provided herein is a method of inhibiting the activity of IL-4 in a biological sample or cell, the method comprising administering to the biological sample or cell an effective amount of a compound described herein or a pharmaceutically acceptable salt, solvate, hydrate, polymorph, co-crystal, tautomer, stereoisomer, isotopically labeled compound, or prodrug thereof, or the pharmaceutical composition described herein, or the therapeutic composition described herein.

In some embodiments, the biological sample or cell is in vivo. In certain embodiments, the biological sample or cell is ex vivo. In some embodiments, the cell is a malignant cell or premalignant cell. In some embodiments, IL-4 is a wild-type cytokine or mutant cytokine.

### EXAMPLES

In order that the present disclosure may be more fully understood, the following examples are set forth. The synthetic and biological examples described in this Application are offered to illustrate the compounds, pharmaceutical compositions, methods, and uses provided herein and are not to be construed in any way as limiting their scope.

### Example 1: Elucidating Structure-Activity Relationships of Small-Molecule IL-4 Inhibition

### Results

### Nico-52 Analog Design and Synthesis

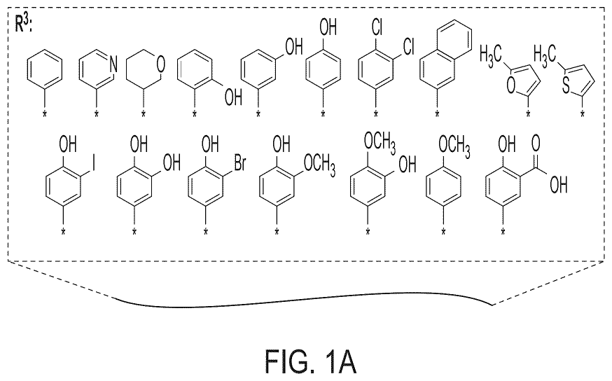

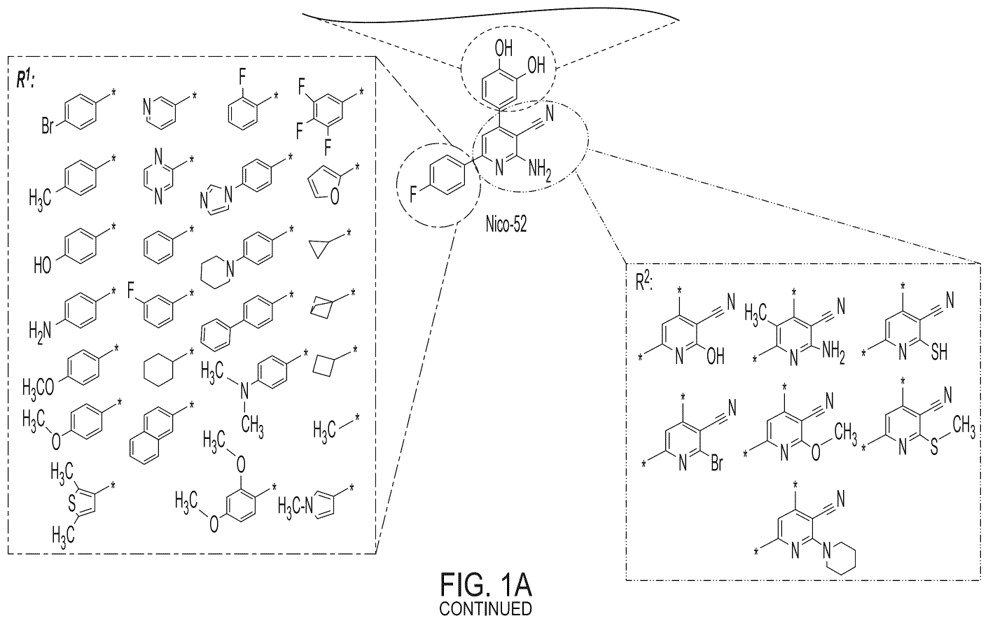

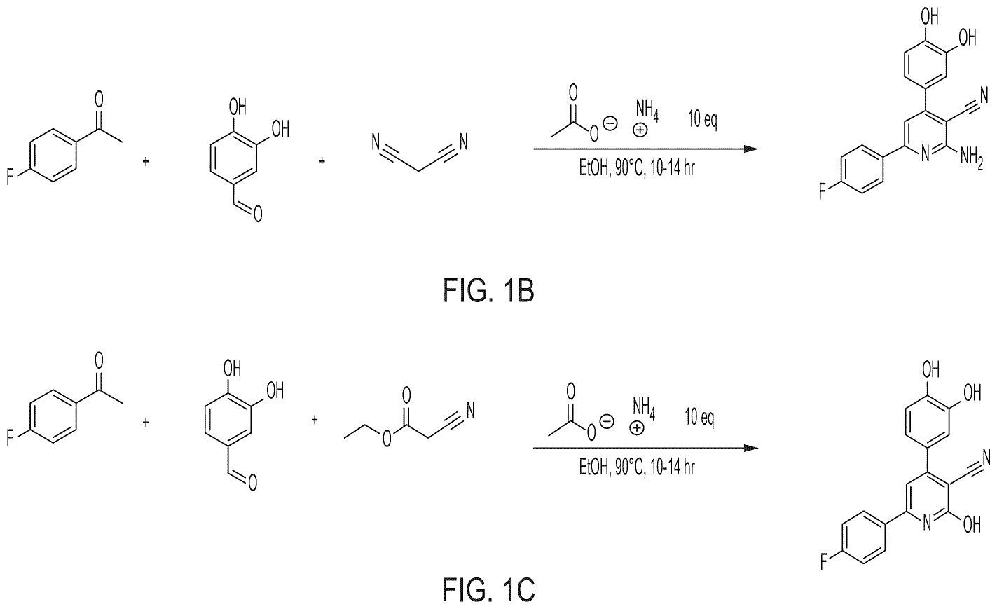

A total of 56 analogs were tested for preliminary inhibition. A three-part model for structural elaboration to determine structure-activity relationships (SAR) was used (FIG. 1A) based on the 3-ring system that comprises the parent compound; the p-fluorophenyl (R¹), the core amino nicotinonitrile (R²), and the ortho-hydroquinone (R³). The synthesis of Nico-52 and its analogs was adapted from Serry et al⁴⁹ which used a one-pot, three-component reaction to combine an acetophenone, an aromatic aldehyde, and malononitrile using ammonium acetate and refluxed in ethanol for 10-14 hours (FIG. 1B). The parent compound Nico-52 was accessed and apply this method widely to synthesize the analogs described here with yields varying based on the components involved. Hydroxy nicotinonitrile R² analogs were synthesized by replacing malononitrile with ethyl cyanoacetate (FIG. 1C).

### Cellular IL-4 Inhibition of Nico-52 Analogs

All Nico-52 analogs were initially evaluated for their IL-4 inhibition using a HEK Blue IL-4/IL-13 reporter assay at a 10 μM concentration (Table 1).^{48,50,51} Since Nico-52 shows near complete inhibition (>97%) at this concentration, analogs could be efficiently grouped as either being at least as potent or less potent than the parent compound. Analogs that showed similar inhibition at 10 μM (>80% inhibition) were then prioritized for a complete dose-response EC₅₀ determination in this reporter system to further determine which analogs possess increased potency over Nico-52. Analogs were then prioritized based on their potency for further testing in non-engineered cell lines for IL-4 inhibition of the type II IL-4R/IL-13R receptor complex (THP-1)^{48,52,53} and type I IL-4R/γ_{c} receptor complex (Ramos).^{54,55} The binding selectivity of top analogs was also evaluated against other members of the IL-4 like cytokine subfamily by differential scanning fluorimetry (DSF, thermal shift) as well as functional inhibition of IL-13, the closest functional homolog to IL-4 which shares the type II receptor. Finally, the potential of analogs for in vivo use was determined with preliminary in vitro ADME/T studies.

It was first investigated the amenability of the para fluoro group of the R¹ position to functional group changes. Analogs with substituted R¹ para positions such as 3 (p-bromine), 4 (p-hydroxy), 5 (p-methyl), and 14 (aniline) exhibited >80% IL-4 percent inhibition. Notably, the substitution of p-fluoro with p-aniline in analog 14 and the removal of the fluoro group altogether in analog 15 resulted in IL-4 inhibition similar to Nico-52, showing near-complete (94.5% and 95.5%, respectively) inhibition. Analog 6, with a substituted p-methoxy group, was evaluated to distinguish the hydrogen bonding effects of the alcohol from analog 4 and showed a potency of 81.7% inhibition.

Next further changes to the aromatic ring were assessed, including heterocycle substitution, naphthyl or bi-phenyl ring systems, or the removal of aromaticity altogether. Substitution of the phenyl ring with a pyridinyl ring retained inhibitory activity (7, 94.3%). The pyridine of 7 retained activity. Removal of aromaticity with the cyclohexane derivative, analog 12, exhibited reduced inhibition to 49.4%. Substitution of the phenyl ring to methyl (13) retained considerable potency (70.2%).

Analogs 16, 17, and 18 (commercially sourced) were evaluated to determine the contribution of pKa changes on the aniline. Certain analogs most of the activity (18). Given these determinations, compounds 3, 4, 5, 6, 7, 14, and 15 were prioritized for dose-response evaluations.

5-methyl amino nicotinonitrile (20) exhibited similar inhibition at 10 μM (97.7%) to the parent compound. These results suggested that the core structure is open to modification at the 2 and 5 positions (Table 1). To further determine the importance of the amino group of the core structure, hydroxy nicotinonitrile analogs like 19 were prepared and evaluated.

This resulted initially in the production of most of the R² analogs and could be compared to their amino nicotinonitrile counterparts. In addition, this allowed access to several R¹ and R³ analogs. As 19 showed high inhibition of IL-4, these changes gave additional insights and reinforce trends seen with the amino nicotinonitrile analogs. Of all the R² hydroxy nicotinonitrile analogs tested only ortho alcohol analog 39 showed comparable inhibition (100%) at 10 μM. Notably, analog 32, featuring p-hydroxy at the R¹ position similar to analog 4, exhibited inhibition comparable to the parent compound at 94.1%.

### Dose-Response of Prioritized Nico-52 Analogs

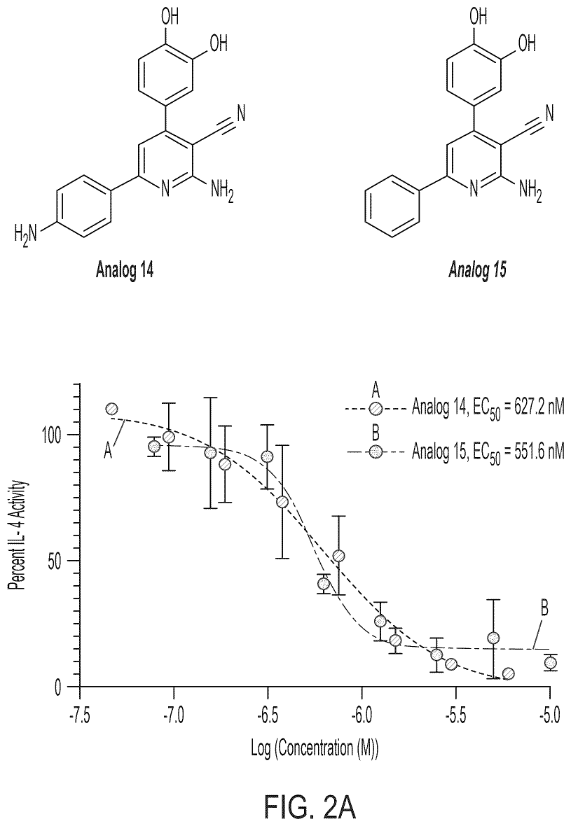

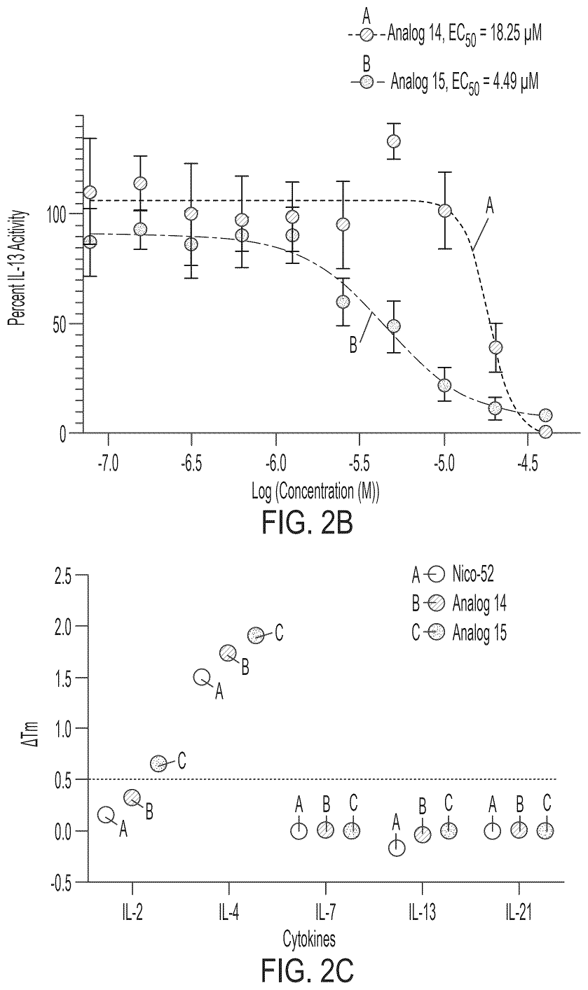

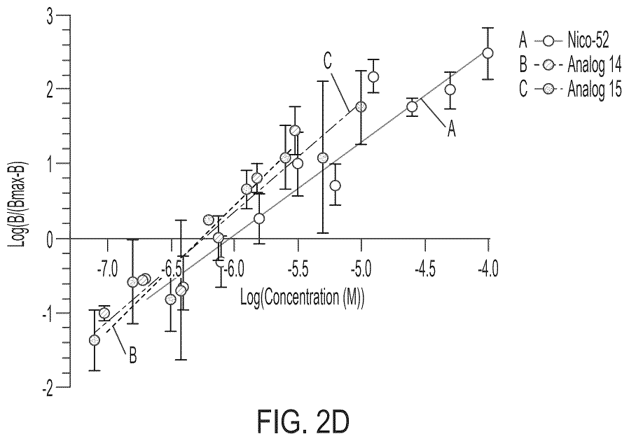

Analogs that showed similar IL-4 inhibition in HEK-Blue IL-4/IL-13 reporter assay to the parent compound at 10 μM were prioritized to determine their EC₅₀ of inhibition (Table 1). Additional prioritization was given to select analogs at certain positions to gain further SAR understanding. Analogs 15 and 14 with modified R¹ positions demonstrated nanomolar potency of 551.6 nM and 627.2 nM respectively (Table 1, FIG. 2A). Whereas other R¹ analogs 5, 6, and 7 with substituted p-methyl, p-methoxy and pyridine showed similar potency to the parent compound Nico-52. These results supported the observation that the R¹ position was tolerant to derivatization. For R² analogs, 20 inhibited IL-4 in the cell reporter assay with less potency than Nico-52. Analog 24, with a substituted methoxy group at the R¹ para position and methyl at the R² 5-position, had a potency of 4.22 μM. Hydroxy nicotinonitrile analog 39 with an R³ ortho alcohol had a potency of 1.75 μM similar to the parent compound. A single hydroxyl could retain potency in the R³ position as the phenol in analog 47 demonstrated a potency of 3.4 μM, likely playing a role in conformational restriction of the axial biphenyl bond. Taken together, derivatives 14 and 15 were identified for further testing.

### Selectivity of Analogs

IL-4 and IL-13 share the same type II receptor for their proinflammatory signaling⁵⁶. This receptor consists of the heterodimerization of IL-4Rα and the IL-13Rα1 subunit and can also be activated by IL-13 binding⁵⁷. Previously, it was reported that Nico-52 had 10-fold higher inhibition for IL-4 activity than IL-13 activity⁴⁸. To determine if analogs 14 and 15 maintained this level of selectivity, they were tested for dose-dependent inhibition of IL-13 activity in the HEK Blue IL-4/IL-13 reporter assay. Analogs 14 and 15 inhibited soluble IL-13 mediated signaling with significantly less potency, with an EC₅₀=18.25 μM (100-fold less than for IL-4) and an EC₅₀=4.49 μM (10-fold less than for IL-4), respectively (FIG. 2B).

IL-4 also mediates signal transduction via a type I receptor complex, which contains IL-4Rα and a common cytokine receptor γ_{c} chain shared by IL-2, IL-7, and IL-21 cytokines⁵⁶. Nico-52 and analogs 14 and 15 were therefore tested for target specificity against the soluble cytokines IL-2, IL-7, IL-21, and IL-13 at a 2 μM final concentration in a thermal shift assay using DSF. Analog 15 specifically binds to soluble IL-4 with a Tₘ shift of 1.75° C., and analog 14 binds to soluble IL-4 with a Tₘ shift of 1.66° C. (FIG. 2C). While analog 15 also bound weakly to soluble IL-2 with a Tₘ shift of 0.66° C., this just barely crossed the 0.5° C. shift threshold to qualify as a binder and the thermal shift for IL-4 was much stronger than IL-2 by over 1° C. Taken together, these results further supported that Nico-52 and analogs 15 and 14 are selective towards soluble IL-4.

For all three compounds. Nico-52, 14, and 15 dose-response data was fitted in GraphPad Prism 9.2.0 using simple linear regression where each data point was normalized based on the maximal response to determine the Hill coefficient, providing insights into the binding dynamics of these compounds. All three compounds showed positive cooperativity with a Hill coefficient >1, suggesting that there may be multiple binding sites on the IL-4 surface. The Hill coefficient for Nico-52 was 1.24±0.08, for analog 14: 1.69±0.25, for analog 15: 1.49±0.15 (FIG. 2D).

Inhibition in Cells that Natively Express the IL-4 Receptor

Further characterization was performed to understand the inhibitory activity and potency of the analogs against cell lines that are not engineered and natively express the IL-4 complexes.

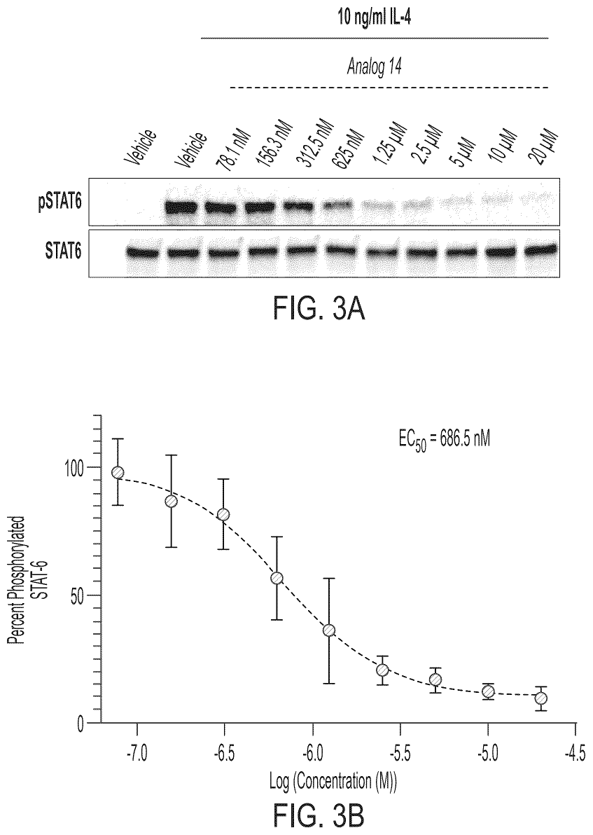

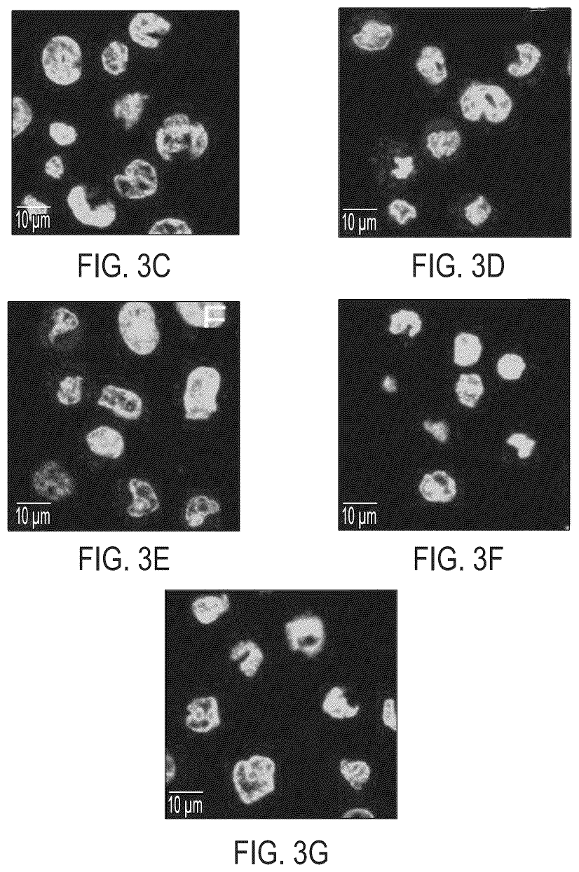

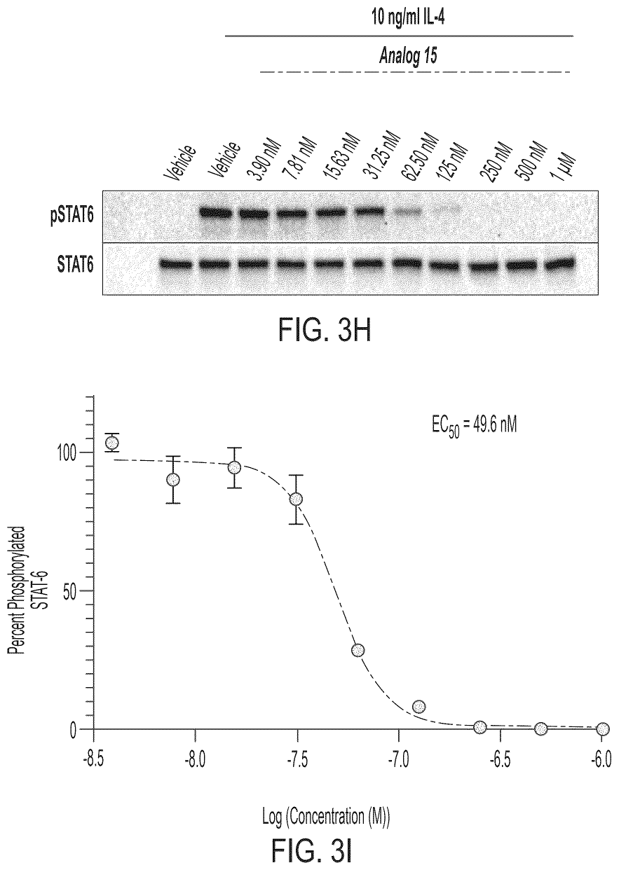

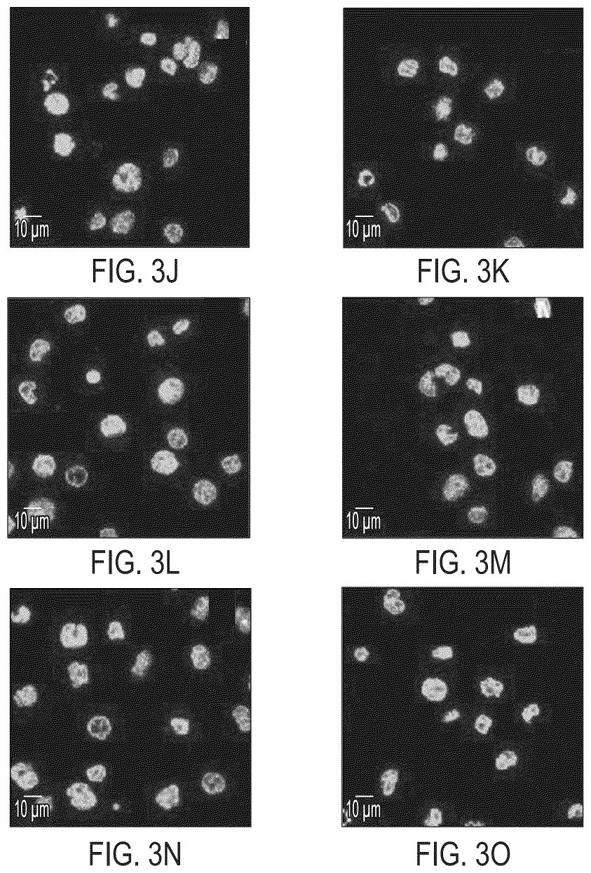

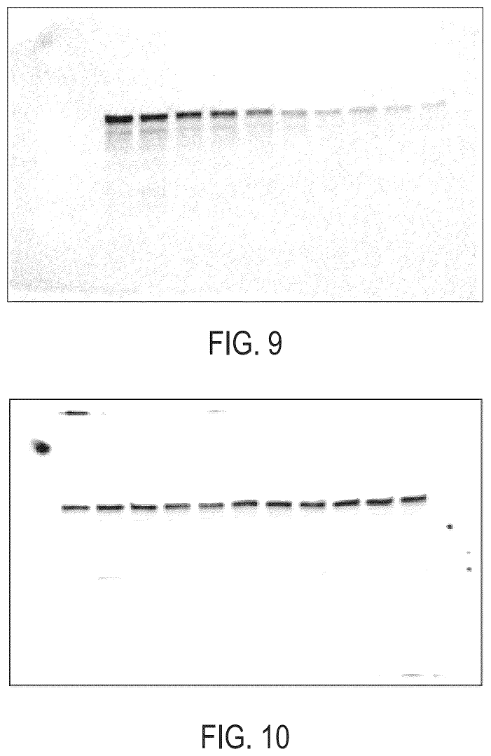

IL-4 binding its receptor in both type I and type II signaling leads to JAK1-mediated STAT-6 phosphorylations⁵². THP-1 monocytes express exclusively the type II IL-4 complex⁵³. To confirm the inhibitory effects of analogs 14 and 15 in this cell line and compare them to previously reported results for Nico-52⁴⁶ (EC₅₀=3.1 μM), THP-1 monocytes were treated with either soluble IL-4 or IL-4 pre-incubated with these analogs in a dose-dependent manner. Levels of STAT-6 phosphorylation were then quantified by western blot to measure pathway activation. Analog 14 inhibited STAT-6 phosphorylation with an EC₅₀=686.5 nM which was consistent with HEK Blue IL-4/IL-13 reporter assay data (FIGS. 3A, 3B, 9, and 10). STAT-6 levels were not impacted by analog 14. To further corroborate these results, analog 14 in a dose-dependent inhibition of IL-4 induced pSTAT-6 levels using immunofluorescence was examined. Treatment of THP-1 cells with IL-4 and analog 14 again demonstrated a significant reduction in STAT-6 phosphorylation at 500 nM and almost no visible STAT-6 phosphorylation at a 5 μM concentration in comparison to IL-4 treated with vehicle (FIGS. 3C-3G).

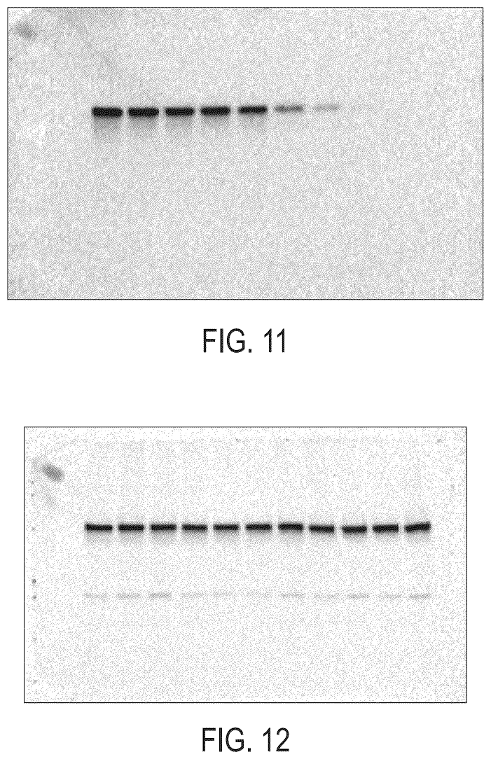

Analog 15 inhibited IL-4 induced STAT-6 phosphorylation with an EC₅₀=49.6 nM which was ˜9 fold more active than what was observed in the HEK Blue IL-4/IL-13 reporter assay (FIGS. 3H, 3I, 11, and 12). STAT-6 levels were not impacted by analog 15. The difference in potency between the engineered reporter system and the THP-1 cells may be attributed to a difference in expression levels of the type 11 receptor complex for the two cell lines. Additionally, THP-1 cells treated with analog 15 were analyzed using immunofluorescence. Treatment of THP-1 cells with analog 15 exhibited reduced STAT-6 phosphorylation at 50 nM and complete inhibition of phosphorylation at 500 nM (FIGS. 3J-3O).

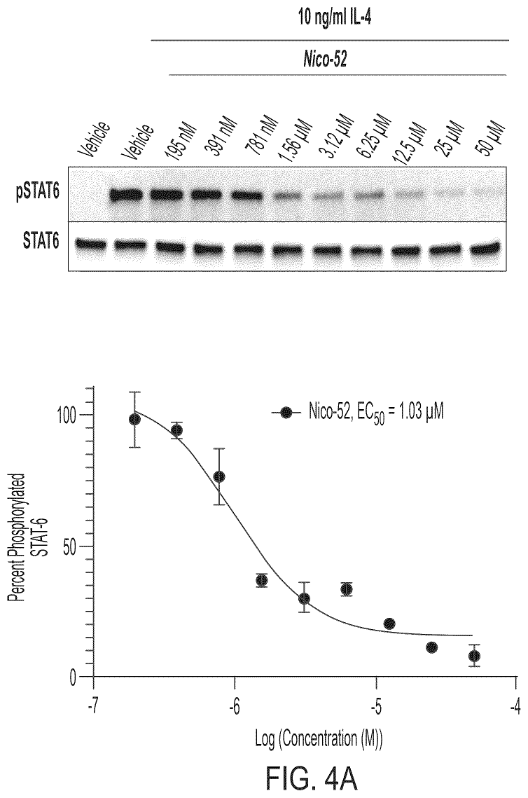

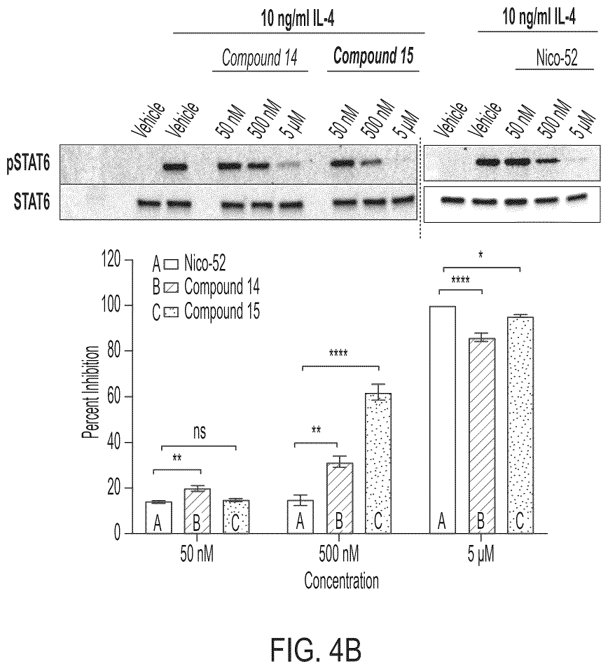

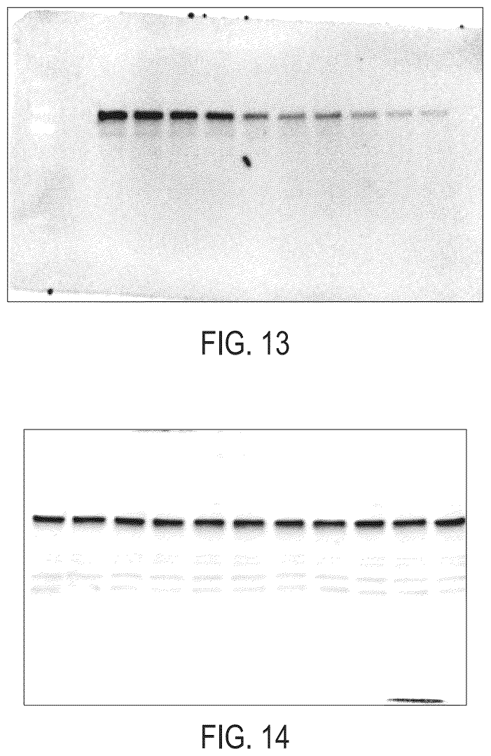

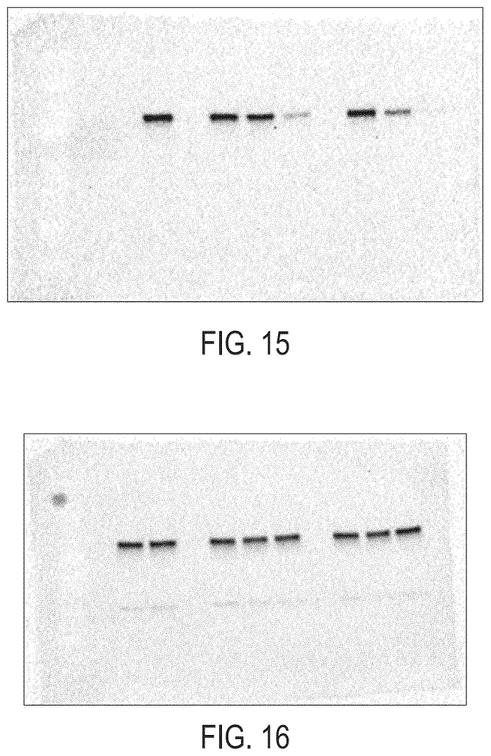

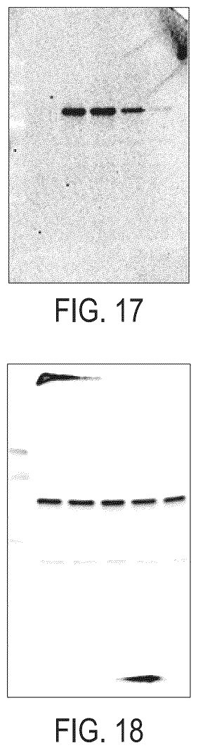

Inhibitory effects of the parent compound (Nico-52) and analogs 14 and 15 were also evaluated in Ramos B lymphocytes which exclusively express the IL-4 type I receptor complex^{54,55}. Ramos cells were exposed to IL-4 with vehicle or Nico-52 in a dose dose-dependent manner, and again pSTAT-6 to STAT-6 levels were quantified by western blot analysis. Nico-52 inhibited IL-4 induced STAT-6 phosphorylation with an EC₅₀=1.03 μM (FIGS. 4A, 13, 14). A comparative evaluation of potency between Nico-52, 14, and 15 was conducted in Ramos B lymphocytes employing a similar method of quantification of pSTAT-6 in relation to STAT-6 as discussed above. At 50 nM, minimum inhibition of phosphorylation of STAT-6 by Nico-52, 14 and 15 was observed. At 500 nM, analog 15 demonstrated a percent inhibition of 61.9%, analog 14 and Nico-52 exhibited a percent inhibition of 31.4% and 14.6% respectively (FIGS. 4B, 15, 16, 17, and 18). At a 5 μM concentration, near complete inhibition was observed for all three compounds. Percent Inhibition at 500 nM was more informative as it revealed that analog 15 demonstrated higher levels of inhibition at 5(0) nM followed by analog 14 and then Nico-52. This result supported that analogs 14 and 15 also have higher potency in Ramos B lymphocytes for type I signaling as well in comparison to Nico-52.

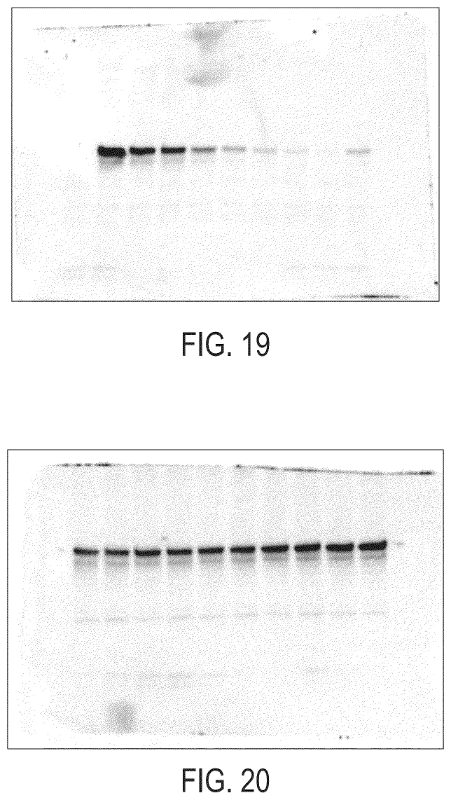

Finally, to evaluate if the compounds disclosed herein are also suitable for the inhibition of murine IL-4 signaling and potential future animal studies in mice, RAW 264.7 macrophages were incubated with murine IL-4 and Nico-52 or vehicle. Dose-dependent inhibition of STAT-6 phosphorylation was measured and quantified via western blotting (FIGS. 19 and 20). Nico-52 exhibits a dose-dependent reduction in pSTAT-6 levels with an EC₅₀ of 1.28 μM. STAT-6 levels were not affected by Nico-52.

### Biophysical Characterization of Analog 15

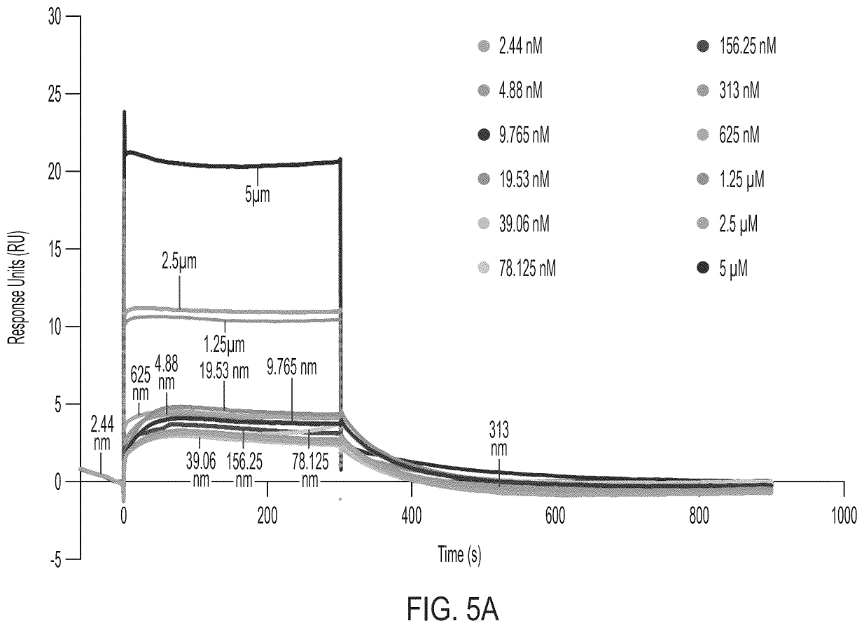

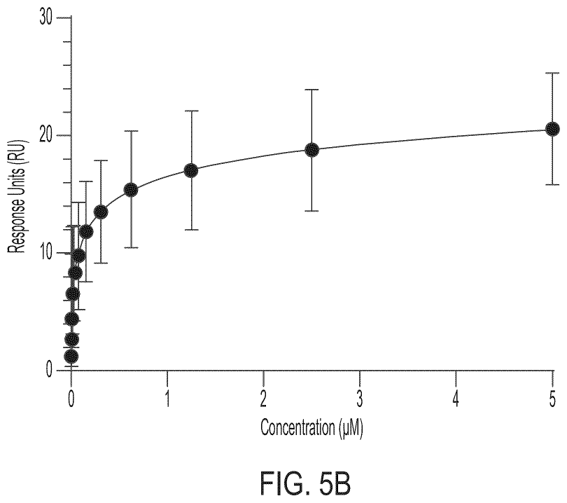

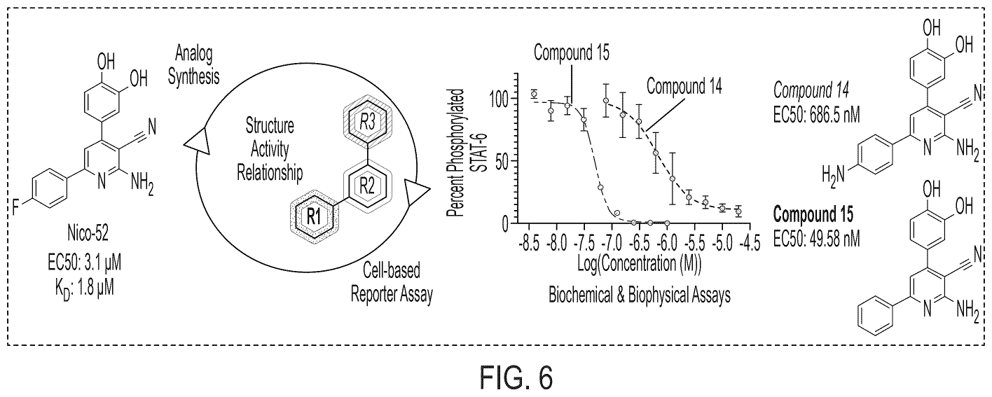

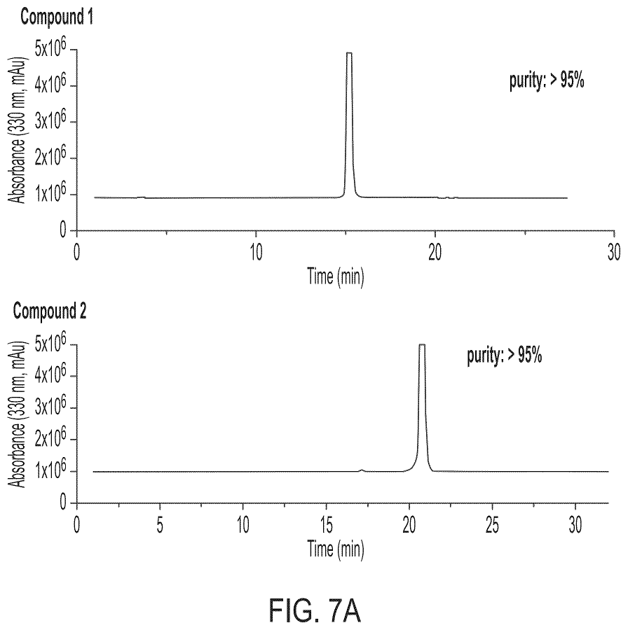

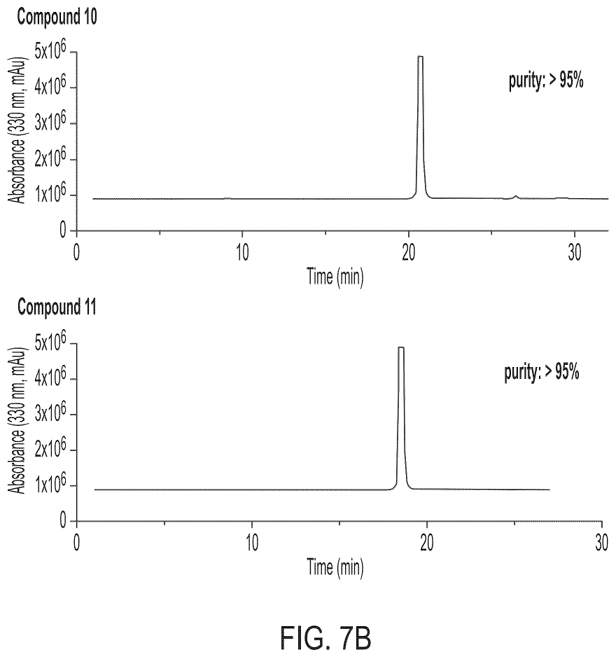

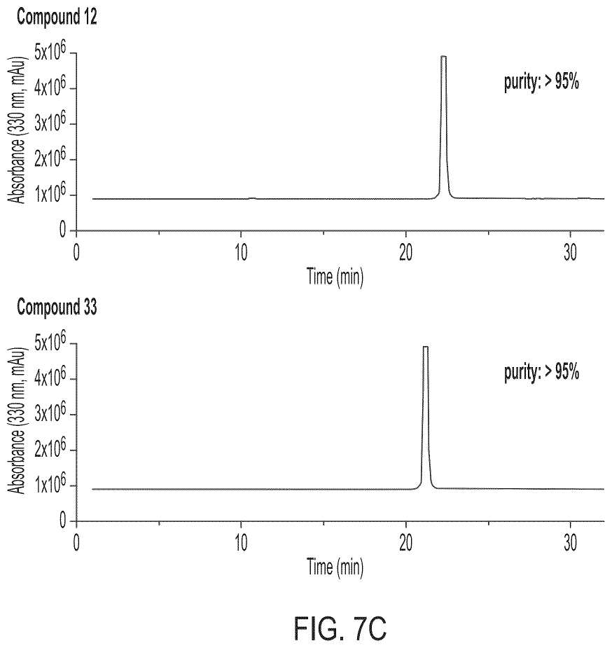

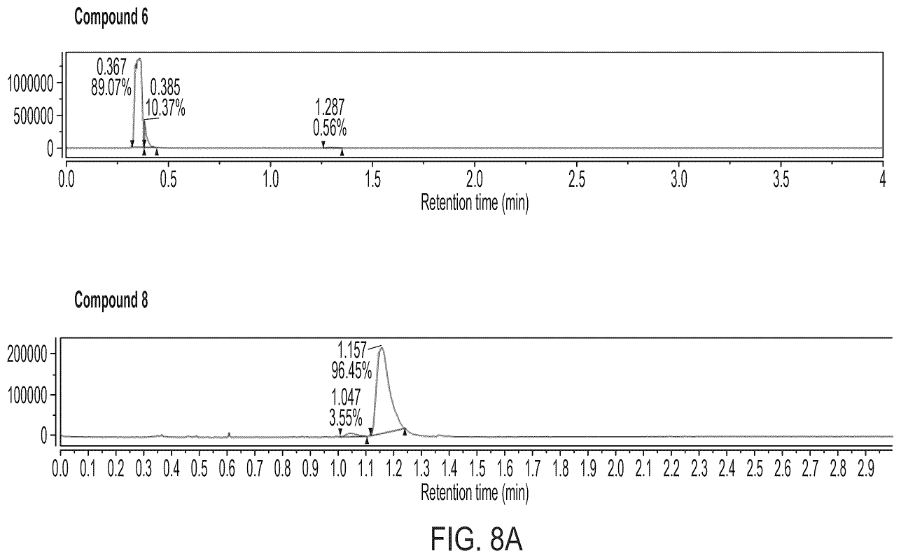

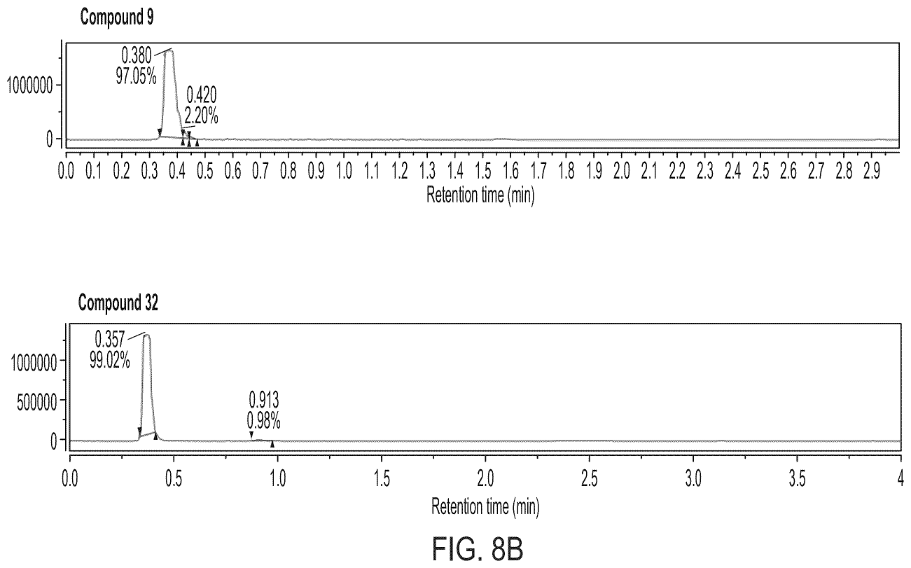

To further characterize the biophysical interactions of analog 15 and IL-4, Surface Plasmon Resonance (SPR) was employed. Sensorgrams generated from SPR binding assays were measured at twelve different concentrations of analog 15, consistent with the dose-dependent inhibition observed in the HEK Blue reporter assay and THP-1 assay (FIG. 5A). The binding profile is consistent with non-covalent, reversible small-molecule binding on the IL-4 surface with distinct association, steady-state, and dissociation phases. Steady-state affinity analysis of 15 exhibited a binding affinity K_{D} of 31.9 nM with kinetic binding constants kₐ=2.62 e+04 M⁻¹s⁻¹ and k_{d}=8.34 e−04 s⁻¹ (FIG. 5B), with steady-state being achieved within 300 seconds. This K_{D} is consistent with the EC₅₀ potency measured in THP-1 cells (49.6 nM), and shows a 57-fold improvement in IL-4 affinity compared to Nico 52 (1.8 μM, previously reported).⁴⁸

TABLE 1
Nico-52 analogs tested at a 10 μM concentration, with prioritized analogs showing
>80% inhibition being evaluated for dose-dependence in the HEK blue IL-4/IL-13 reporter
assay. For each analog, the structural change made at each position (R¹, R², or R³) is
indicated. Blank spaces indicate that structural position matches that of the parent compound
Nico52. For analogs that were comparably to Nico-52 at 10 μM, the EC50 was determined
and the coefficient of de-termination (r2) is reported in Table 5.

| | | | | % | EC50 |
|---|---|---|---|---|---|
| ID | R¹ | R² | R³ | Inhibition | (μM) |
| Nico-52 | 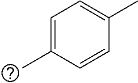 | 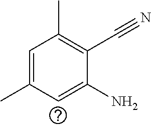 | 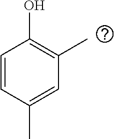 | 97.6 ± 1.57% | 1.32 ± 0.38 |
| 1 | 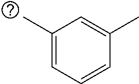 | | | No inhibition | |
| 2 | 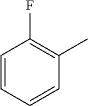 | | | 40.0 ± 5.87% | |
| 3 | 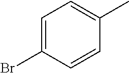 | | | 63.3 ± 2.54% | 1.30 ± 0.39 |
| 4 | 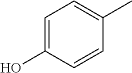 | | | 81.2 ± 18.9% | 1.14 ± 0.38 |
| 5 | 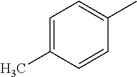 | | | 62.7 ± 4.30% | 1.29 ± 0.47 |
| 6 | 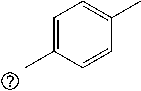 | | | 51.7 ± 5.05% | 3.02 ± 0.61 |
| 7 | 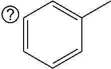 | | | 84.3 ± 1.11% | 2.02 ± 0.97 |
| 8 | 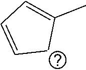 | | | No inhibition | |
| 9 | 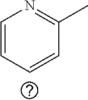 | | | 64.2 ± 13.4% | |
| 10 | 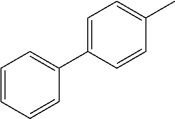 | | | 4.73 ± 4.58% | |
| 11 | 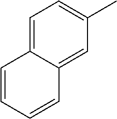 | | | 54.7 ± 8.42% | |
| 12 | 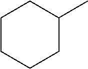 | | | 48.4 ± 5.80% | |
| 13 | 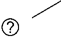 | | | 70.2 ± 7.84% | |
| 14 | 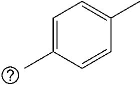 | | | 94.5 ± 1.58% | 0.67 ± 0.28 |
| 15 | 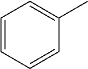 | | | 95.5 ± 0.36% | 0.85 ± 0.12 |
| 16 | 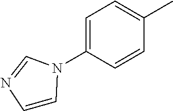 | | | No inhibition | |
| 17 | 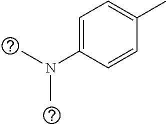 | | | 22.9 ± 1.48% | |
| 18 | 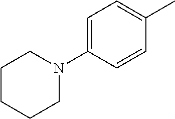 | | | 88.2 ± 13.9% | |
| 19 | | 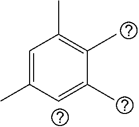 | | 71.2 ± 5.60% | |
| 20 | | 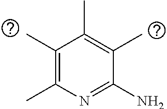 | | 97.7 ± 0.59% | 2.69 ± 0.57 |
| 21 | 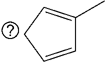 | | 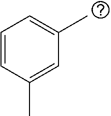 | 24.1 ± 8.09% | |
| 22 | 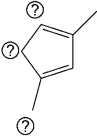 | | 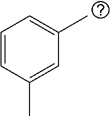 | 60.2 ± 10.2% | |
| 23 | 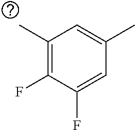 | | 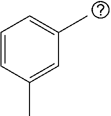 | 67.6 ± 5.85% | |
| 24 | 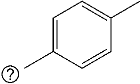 | 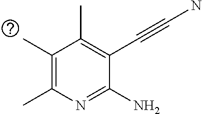 | | 95.8 ± 0.67% | 4.22 ± 0.71 |
| 25 | 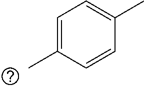 | 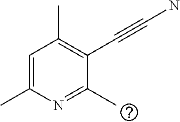 | 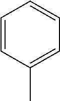 | 83.5 ± 14.7% | |
| 26 | 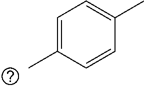 | 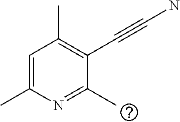 | 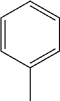 | 79.5 ± 10.8% | |
| 27 | 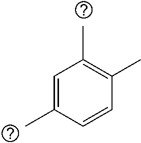 | | 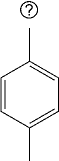 | 78.8 ± 16.6% | |
| Nico-52 | 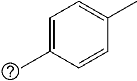 | 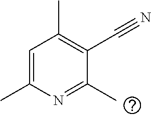 | 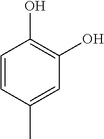 | 97.6 ± 1.57% | 1.32 ± 0.36 |
| 28 | 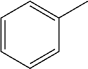 | 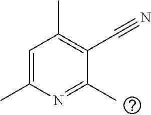 | 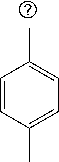 | 61.4 ± 8.46% | |
| 29 | 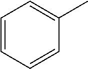 | 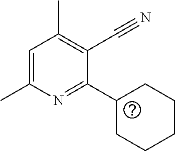 | 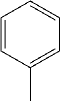 | 59.8 ± 20.1% | |
| 30 | 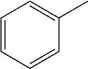 | 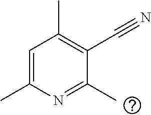 | 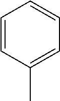 | 50.4 ± 2.82% | |
| 31 | 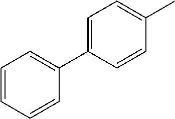 | 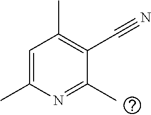 | | 68.7 ± 9.84% | |
| 32 | 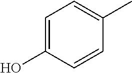 | 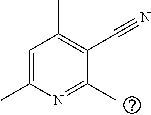 | | 94.1 ± 1.65% | |
| 33 | 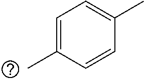 | | 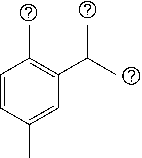 | 6.63 ± 0.00% | |
| 34 | | 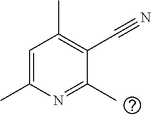 | 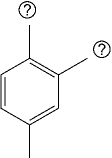 | 44.2 ± 11.1% | |
| 35 | | 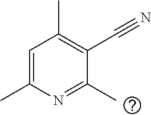 | 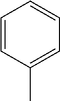 | 27.6 ± 25.1% | |
| 36 | | 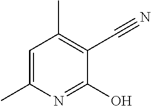 | 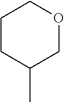 | 31.7 ± 21.6% | |
| 37 | | 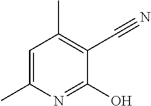 | 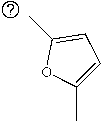 | 0.48 ± 17.1% | |
| 38 | | 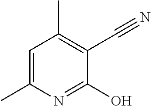 | 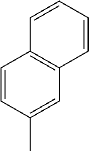 | 51.8 ± 17.6% | |
| 39 | | 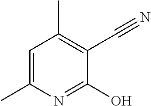 | 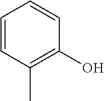 | 100 ± 1.87% | 1.75 ± 0.55 |
| 40 | | 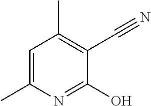 | 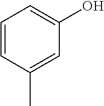 | 64.3 ± 12.3% | |
| 41 | | 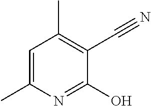 | 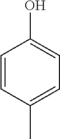 | 60.7 ± 10.3% | |
| 42 | | 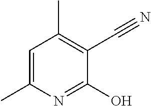 | 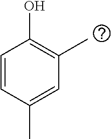 | 67.6 ± 4.88% | |
| 43 | | | 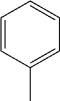 | 6.13 ± 9.13% | |
| 44 | | | 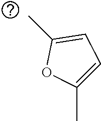 | 33.9 ± 21.4% | |
| 45 | | | 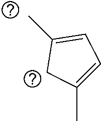 | 37.5 ± 19.9% | |
| 46 | | | 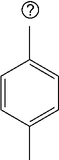 | 47.2 ± 12.1% | |
| 47 | | | 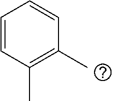 | 98.8 ± 1.54% | 3.45 ± 0.92 |
| 48 | | | 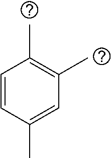 | 100 ± 0.49% | 8.40 ± 0.76 |
| 49 | | | 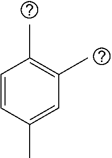 | 82.2 ± 7.20% | 4.02 ± 1.85 |
| 50 | | | 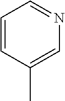 | 10.0 ± 17.2% | |
| 51 | | | 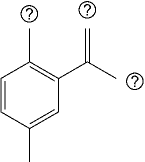 | 54.6 ± 11.6% | |
| 52 | | | 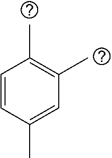 | 58.8 ± 16.5% | |
| 53 | | | 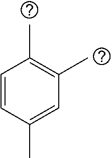 | 58.9 ± 19.9% | |

TABLE 5
Coefficient of determination (r²) values for the Nico-52 analogs which were further
evaluated for dose response in HEK Blue IL-4/IL-13 reporter cell line.

| ID | R¹ | R² | R³ | % Inhibition | EC50 (μM) | r² |
|---|---|---|---|---|---|---|
| Nico-52 | 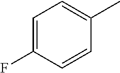 | 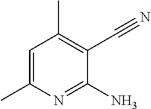 | 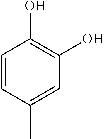 | 97.6 ± 1.57% | 1.32 ± 0.38 | 0.92 |
| 3 | 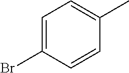 | | | 93.3 ± 2.54% | 1.30 ± 0.39 | 0.90 |
| 4 | 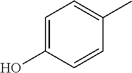 | | | 81.2 ± 18.9% | 1.14 ± 0.39 | 0.85 |
| 5 | 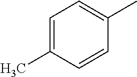 | | | 82.7 ± 4.30% | 1.29 ± 0.47 | 0.82 |
| 6 | 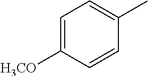 | | | 81.7 ± 8.05% | 3.02 ± 0.81 | 0.87 |
| 7 | 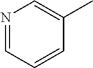 | | | 94.3 ± 1.11% | 2.02 ± 0.67 | 0.78 |
| 14 | 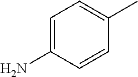 | | | 94.5 ± 1.68% | 0.67 ± 0.26 | 0.93 |
| 15 | 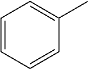 | | | 95.5 ± 0.36% | 0.55 ± 0.12 | 0.92 |
| 20 | | 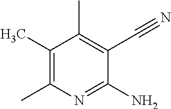 | | 97.7 ± 0.59% | 2.69 ± 0.57 | 0.93 |
| 24 | 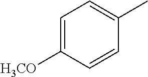 | 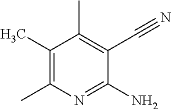 | | 96.8 ± 0.87% | 4.22 ± 0.71 | 0.94 |
| 39 | | 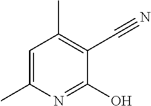 | 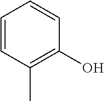 | 100 ± 1.87% | 1.75 ± 0.55 | 0.87 |
| 47 | | | 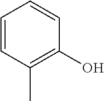 | 98.8 ± 1.54% | 3.45 ± 0.92 | 0.83 |
| 48 | | | 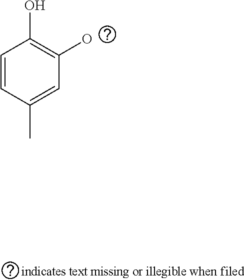 | 100 ± 0.49% | 6.40 ± 0.76 | 0.95 |
| 49 | | | 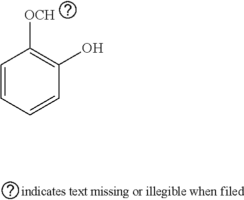 | 82.2 ± 7.20% | 4.02 ± 1.85 | 0.79 |

In vitro ADME/T of Certain Analogs

Nico-52, analogs 14 and 15 were evaluated for cytotoxicity in B16-F10 cells at 25 μM, revealing no toxicity after a 24-hour incubation period. Following this, metabolic stability assessments utilizing mouse hepatic microsomes indicated that after one hour, Nico-52 and 14 demonstrated retention of 86.5% and 75.4% of their initial concentrations, respectively.

All three compounds displayed stability in both mouse and human plasma. After three hours, 97% of Nico-52 was retained in mouse plasma and >99% in human plasma. Analog 14 displayed >99% retention in both mouse and human plasma. Similarly, analog 15 also exhibited significant retention with 94.3% in mouse plasma and 97.4% in human plasma. PAMPA assays unveiled distinct permeability coefficients comparable to other membrane-permeable, orally bioavailable compounds, further highlighting the potential of this scaffold (Table 2).

TABLE 2
In vitro ADME/T of Nico-52 and certain analogs. Nico-52, 14 and 15
cytotoxicity evaluation at 25 μM in B16-F10 cells for 24 hours.
All three compounds were also tested for mouse hepatic microsome
stability, mouse & human plasma stability, and PAMPA permeability

| ADME/T | Nico-52 | Analog 14 | Analog 15 |
|---|---|---|---|
| Cytotoxicity in B16-F10 cells (24 hours) | n.s. < 25 μM | n.s. < 25 μM | n.s. < 25 μM |
| Mouse hepatic microsome stability | 86.5% | 75.4% | 30.3% |
| (% remaining after 1 hour) | | | |
| Mouse/Human plasma stability | 97%/>99% | >99%/>99% | 94.3%/97.4% |
| (% remaining affer 3 hours) | | | |
| PAMPA Permeability | 16.4 | 6.2 | 12.7 |
| (×10^{-6 cm/s}) | | | |

Closer examination of the LC/MS profile from the microsomal stability assay supported oxidation (hydroxylation) of analog 15 (data not shown). Only the R¹ position varied between 15 and the parent compound, so it was hypothesized that implementing a bioisostere for the phenyl group at this position could improve metabolic stability. The phenyl ring at R¹ was substituted with three bioisosteres: bicyclopentanyl (1A/54), cyclopropyl (2A/55), and cyclobutyl (3A/56). It was then first verified if these new analogs retained some of the potency of 15. Analog 1A/54 with the bicyclopentane moiety exhibited inhibition of 86.1% at 10 μM in the HEK Blue IL-4/IL-13 reporter assay (Table 3), 1A/54 showed improved metabolic stability compared to 15 with 68.3% remaining after an hour upon treatment with mouse hepatic microsomes (Table 3).

TABLE 3
Preliminary optimization of analog 15 for metabolic stability. Analogs of 15 with
phenyl group bioisosteres at the R¹ position were first evaluated for IL-4 inhibition in the
HEK Blue IL-4/IL-13 reporter assay at 10 μM. Having retained activity, analog 54 was
further evaluated metabolic stability in the mouse hepatic microsomal stability assay.

| Compound | Structure | % Inhibition |
|---|---|---|
| Nico-52 | 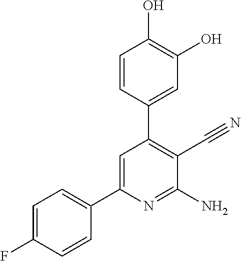 [SMILES: N#Cc1c(-c2ccc(O)c(O)c2)cc(-c2ccc(F)cc2)nc1N] | 97.6 ± 1.57% |
| 1A/54 | 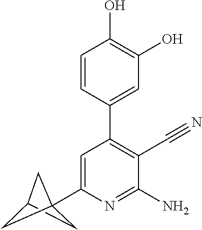 [SMILES: N#Cc1c(-c2ccc(O)c(O)c2)cc(C23CC(C2)C3)nc1N] | 86.1 ± 10.7% |
| 2A/55 | 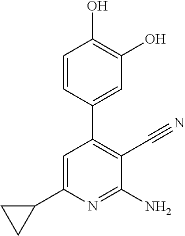 [SMILES: N#Cc1c(-c2ccc(O)c(O)c2)cc(C2CC2)nc1N] | 2.47 ± 14.2% |
| 3A/56 | 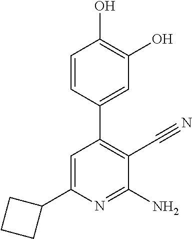 [SMILES: N#Cc1c(-c2ccc(O)c(O)c2)cc(C2CCC2)nc1N] | 28.5 ± 12.1% |
| 4A | 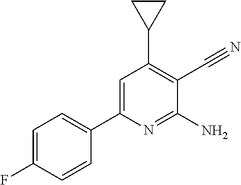 [SMILES: N#Cc1c(C2CC2)cc(-c2ccc(F)cc2)nc1N] | |
| 5A | 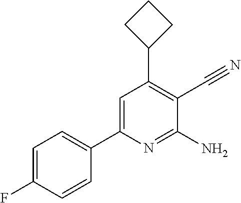 [SMILES: N#Cc1c(C2CCC2)cc(-c2ccc(F)cc2)nc1N] | |
| 6A | 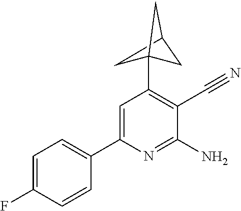 [SMILES: N#Cc1c(C23CC(C2)C3)cc(-c2ccc(F)cc2)nc1N] | |

Analogs of Nico-52 with phenyl group bioisosteres at the R¹ position were first evaluated for IL-4 inhibition in the HEK Blue IL-4/IL-13 reporter assay at 10 μM. Having retained activity, analog 1A was further evaluated metabolic stability in the mouse hepatic microsomal stability assay. To increase metabolic stability, the bioisosteres were installed at the R¹ and/or R² positions. Three bioisosteres for phenyl rings, bicyclopentanyl (1A/54), cyclopropyl (2A/55), and cyclobutyl (3A/56) were implemented. Notably, compound 1A/54 with the bicyclopentanyl moiety exhibited inhibition of 86.1% at 10 μM in the IL-4/IL-13 HEK Blue reporter cell line (Table 3). Compound 1A/54 showed metabolic stability with 68.3% remaining after an hour upon treatment with mouse hepatic microsomes.

In summary, the studies of the Nico-52 scaffold revealed insights into designing enhanced inhibitors of the soluble cytokine IL-4. As Nico-52 was the first reported inhibitor of IL-4, the analogs 14 and 15 are now the first reported nanomolar IL-4 inhibitors. Furthermore, these results contribute to further characterizing and understanding chemotypes capable of inhibiting protein-protein interaction with no defined binding grooves/pockets like the interaction between IL-4 and its receptors⁵⁸. Blocking IL-4-induced immune responses has improved lung function in mouse models of pulmonary inflammation^{59,60} and impacts macrophage polarization in tumor models⁶¹. Finally, the advent of a small molecule inhibitor of soluble IL-4 with nanomolar potency can complement other cytokine-targeted therapeutic modalities.

### Nico-52 Analog Design and Synthesis

Compounds of the present disclosure were synthesized using a procedure adapted from Serry et al⁴⁹ which used a one-pot, three-component reaction to combine an acetophenone, an aromatic aldehyde, and malononitrile using ammonium acetate and refluxed in ethanol for 10-14 hours.

### Example 2: Experimental

General Information for Nico-52 Analog Synthesis. The Perkin Elmer Signal notebook was used for experimental procedure planning. All reactions were performed in 1-dram vials unless otherwise noted. Analytical thin layer chromatography (TLC) was performed using 0.25 mm silica gel 60-F plates (Silicycle, Inc.). All compounds were purified using a CombiFlash automated column from Teledyne ISCO on RediSep Rf or Rf Gold silica Flash columns, 20-40 microns unless otherwise noted.

All ¹H NMR spectra were recorded at 400 or 500 MHz at ambient temperature with CDCl₃, CD₃OD, or DMSO (d₆) as the solvent. Chemical shifts are recorded in parts per million (ppm) relative to CDCl₃ (¹H, δ 7.26; ¹³C, δ 77.1), CD₃OD (¹H, δ 3.31; ¹³C, δ 49.0), or (CD₃)₃SO (¹H, δ 2.50; ¹³C, δ 39.5), unless otherwise stated. Data for ¹H NMR are reported as follows: chemical shift, multiplicity (s=singlet; d=doublet; t=triplet; q=quartet, m=multiplet; br=broad; ovrlp=overlapping), coupling constants (J values), and integration. Analytical LCMS was performed on a Waters Acquity UPLC (Ultra Performance Liquid Chromatography (Waters MassLynx Version 4.1) with a Binary solvent manager, SQ mass spectrometer, Water 2996 PDA (PhotoDiode Array) detector, and ELSD (Evaporative Light Scattering Detector).

Synthesis of Nico-52 and Nico-52 derivatives. In a 4-dram pressure seal vial, a solution of ammonium acetate (963.5 mg, 10 Eq, 12.5 mmol) in 15 mL ethanol was added with R¹ ketone derivative (1.0 Eq, 1.25 mmol) and stirred for 5 minutes. Then R³ aldehyde derivative (1.0 Eq, 1.25 mmol) was added and stirred for 5 minutes. Lastly, malononitrile (82.58 mg, 1.0 Eq, 1.25 mmol) or ethyl 2-cyanoacetate (141.4 mg, 133 μL, 1.0 Eq, 1.25 mmol) was added, and the reaction vial was sealed and heated at 90° C. for 10-14 hours. After completion, the reaction was cooled to room temperature, the vial seal was removed and the stirring stopped. The vial was allowed to slowly evaporate the solvent in the hood until a brown-yellow precipitate formed or until half the solvent remained. If a precipitate formed, filtration was performed and the solid was washed with either cold ethanol or toluene and air dried. If the precipitate was not pure by NMR, recrystallization from either MeOH or 1:10 DMF:EtOH.

If no precipitate formed, the reaction was loaded onto celite in a pre-column cartridge that contained silica gel and placed under vacuum for 48 hours prior to column chromatography. For all column chromatography, 100% DCM was run for 10 minutes, then a 35-minute ramp to 15 percent MeOH, and then a 10-minute flush at 40 percent MeOH was run. UPLC was performed to identify the product and the solvent was evaporated to afford Nico-52 derivative product. NMR in DMSO-d was used to determine purity and HPLC was performed for additional purification using an ACN: H₂O gradient. All compounds are >95% pure by HPLC.

Nico-52: 4-(3,4-dihydroxyphenyl)-6-(4fluorophenyl)-2-imino-1,2-dihydropyridine-3-carbonitrile, yellow-brown solid, Yield: 13% (150 mg), 1H NMR (500 MHz, DMSO-d) δ 9.47 (s, 1H), 9.27 (s, 1H), 8.20-8.14 (m, 2H), 7.74-7.64 (m, 1H), 7.35-7.27 (m, 2H), 7.18 (s, 1H), 7.09 (d, J=2.3 Hz, 1H), 7.00 (d, J=2.2 Hz, OH), 6.88 (d, J=8.2 Hz, 3H). MS (EI) m/z: 322 (M++H).

Compound 1: 2-amino-4-(3A-dihydroxyphenyl)-6-(3-fluorophenyl)nicotinonitrile. ¹H NMR (500 MHz, DMSO) δ 7.94 (m, 2H), 7.55-7.47 (m, 1H), 7.32-7.27 (t, 1H), 7.23 (s, 1H), 7.08 (s, 1H), 6.99 (d, J=5.9 Hz, 1H), 6.94 (s, 2H), 6.86 (d, J=8.2 Hz, 1H). UPC²-MS (ES+) m/z:322 (M++H).

Compound 2: 2-amino-4-(3A-dihydroxyphenyl)-6-(2-fluorophenyl)nicotinonitrile. ¹H NMR (500 MHz, DMSO) δ 7.92 (td. J=8.0, 1.9 Hz, 1H), 7.55-7.47 (m, 1H), 7.38-7.27 (m, 2H), 7.04 (d, J=2.2 Hz, 1H), 7.00-6.93 (m, 4H), 6.87 (d, J=8.2 Hz, 1H). UPC²-MS (ES+) m/z:322 (M++H).

Compound 3: 6-(4-bromophenyl)-4-(3,4-dihydroxyphenyl)-2-imino-2,3-dihydropyridine-3-carbonitrile, yellow-brown solid. ¹H NMR (500 MHz, CDCL3) δ 8.04 (d, J=8.6 Hz, 2H), 7.66 (d, J=8.6 Hz, 2H), 7.18 (s, 1H), 7.05 (d, J=2.2 Hz, 1H), 6.96 (dd, J=8.2, 2.3 Hz, 1H), 6.84 (d, J=8.2 Hz, 1H). MS (EI) m/z: 382 (M++H).

Compound 4: 4-(3,4-dihydroxyphenyl)-6-(4-hydroxyphenyl)-2-imino-1,2-dihydropyridine-3-carbonitrile, yellow-brown solid, Yield: 2% (8 mg). ¹H NMR (500 MHz, DMSO-d) δ 8.01-7.95 (m, 2H), 7.14-7.06 (m, 2H), 6.99 (dd. J=8.1, 2.3 Hz, 1H), 6.93-6.84 (m, 3H), 6.78 (s, 2H). MS (EI) m/z: 320 (M++H).

Compound 5: 4-(3,4-dihydroxyphenyl)-2-imino-6-(p-tolyl)-1,2-dihydropyridine-3-carbonitrile, yellow-brown solid, 6% (22 mg). ¹H NMR (500 MHz, DMSO-d) δ 9.48 (s, 3H), 8.16-8.11 (m. OH), 8.04-7.98 (m, 2H), 7.29 (d, J=8.0 Hz, 2H), 7.15 (s, 1H), 7.08 (d, J=2.2 Hz, 1H), 6.98 (dd, J=8.2, 2.2 Hz, 1H), 6.87 (d, J=8.5 Hz, 3H), 2.36 (s, 3H). MS (EI) m/z: 318 (M++H).

Compound 6: 4-(3,4-dihydroxyphenyl)-2-imino-6-(4-methoxyphenyl)-2,3-dihydropyridine-3-carbonitrile, yellow solid, ¹H NMR (500 MHz, CDCL3) δ 8.05 (d, J=8.9 Hz, 2H), 7.09 (s, 1H), 7.04 (d, J=2.2 Hz, 1H), 7.00 (d, J=8.9 Hz, 2H), 6.94 (dd, J=8.1, 2.2 Hz, 1H), 6.84 (d, J=8.2 Hz, 1H), 3.79 (s, 3H). MS (EI) m/z: 334 (M++H).

Compound 7: 4-(3,4-dihydroxyphenyl)-6-imino-1,6-dihydro-[2,3′-bipyridine]-5-carbonitrile, yellow-brown solid Yield: 3% (13 mg). ¹H NMR (500 MHz, DMSO-d) δ 9.41 (d, J=2.1 Hz, 1H), 8.86 (t, J=7.9 Hz, 2H), 7.89 (dd, J=8.1, 5.3 Hz, 1H), 7.41 (s, 1H), 7.30 (s, 1H), 7.22 (d, J=5.0 Hz, 1H), 7.12 (d, J=2.0 Hz, 2H), 7.06-7.00 (m, 2H), 6.90 (d, J=8.2 Hz, 1H), 6.78-6.61 (m, 2H). MS (EI) m/z: 305 (M++H).

Compound 8: 2-amino-4-(3,4-dihydroxyphenyl)-6-(furan-2-yl)nicotinonitrile. Light yellow solid. Yield: 7.09% (30.1 mg). ¹H NMR (500 MHz, dmso) δ 7.86 (s, 1H), 7.16 (d, J=3.7 Hz, 1H), 7.02 (d. J=2.1 Hz, 1H), 6.96 (s, 1H), 6.93 (dd, J=8.2, 2.2 Hz, 1H), 6.88 (s, 1H), 6.85 (d, =8.2 Hz, 1H). MS (EI) m/z: 294 (M++H).

Compound 9: 4-(3,4-dihydroxyphenyl)-2-imino-6-(pyrazin-2-yl)-2,3-dihydropyridine-3-carbonitrile. Light yellow solid, Yield: 0.26% (1.7 mg). ¹H NMR (500 MHz, CDCL₃) δ 7.91 (dd, J=8.6, 5.3 Hz, 2H), 7.34 (t, J=8.7 Hz, 2H), 7.29 (s, 1H), 7.07 (d. J=8.2 Hz, 1H), 6.78 (s, 1H). MS (EI) m/z: 306 (M++H).

Compound 10: 6-([1,1′-biphenyl]-4-yl)-4-(3,4-dihydroxyphenyl)-2-imino-1,2-dihydropyridine-3-carbonitrile, yellow-brown solid, Yield: 1% (5 mg). ¹H NMR (5(0) MHz, CD₃OD) δ 8.15-8.09 (m, 2H), 7.73-7.69 (m, 2H), 7.69-7.64 (m, 2H), 7.45 (dd, J=8.4, 6.9 Hz, 3H), 7.38-7.32 (m, 1H), 7.20 (s, 1H), 7.14 (d, J=2.3 Hz, 1H), 7.05 (dd, J=8.2, 2.2 Hz, 1H), 6.91 (d, J=8.2 Hz, 1H). MS (EI) m/z: 380 (M++H).

Compound 11: 4-(3,4-dihydroxyphenyl)-2-imino-6-(naphthalen-2-yl)-1,2-dihydropyridine-3-carbonitrile, yellow-brown solid, Yield: 1% (4 mg). ¹H NMR (500 MHz, CD3OD) δ 8.59-8.49 (m, 1H), 8.20-8.10 (m, 1H), 8.02-7.84 (m, 4H), 7.54-7.48 (m, 2H), 7.30 (s, 1H), 7.16 (d, J=2.2 Hz, 1H), 7.07 (dd. J=8.2, 2.2 Hz, 1H), 6.92 (d, J=8.2 Hz, 1H). MS (EI) m/z: 354 (M++H).

Compound 12: 2-amino-6-cyclohexyl-4-(3,4-dihydroxyphenyl)nicotinonitrile. ¹H NMR (500 MHz, DMSO) δ 9.32 (s, 2H), 6.96 (d, J=2.2 Hz, 1H), 6.89-6.78 (m, 2H), 6.66 (s, 2H), 6.47 (s, 1H), 2.48 (d, J=2.3 Hz, 1H), 1.81-1.71 (m, 4H), 1.67 (d, J=12.8 Hz 1H), 1.45 (qd. J=13.0, 3.7 Hz, 2H), 1.36-1.25 (m, 2H), 1.23-1.13 (m, 1H). UPC²-MS (ES+) m/z: 310 (M++H).

Compound 13: 2-amino-4-(3,4-dihydroxyphenyl)-6-methylnicotinonitrile. Light yellow solid, 1.65% (10.3 mg). ¹H NMR (500 MHz, DMSO) δ 6.94 (d, J=2.1 Hz, 1H), 6.86 (dd, J=8.4, 2.0 Hz, 1H), 6.82 (d, J=8.2 Hz, 1H), 6.53 (s, 1H), 2.25 (s, 3H). MS (EI) m/z: 242 (M++H).

Compound 14: 6-(4-aminophenyl)-4-(3,4-dihydroxyphenyl)-2-imino-1,2-dihydropyridine-3-carbonitrile, yellow-brown solid, Yield: 3% (11 mg). ¹H NMR (500 MHz, DMSO-d) δ 9.36 (s, 2H), 7.87-7.81 (m, 2H), 7.03 (d, J=2.2 Hz, 1H), 6.98 (s, 1H), 6.94 (dd, J=8.2, 2.2 Hz, 1H), 6.86 (d, J=8.2 Hz, 1H), 6.64 (s, 2H), 6.63-6.57 (m, 2H), 5.62 (s, 2H). MS (EI) m/z: 319 (M++H).

Compound 15: 4-(3,4-dihydroxyphenyl)-2-imino-6-phenyl-1,2-dihydropyridine-3-carbonitrile, yellow-brown solid, Yield: 6% (24 mg). ¹H NMR (500 MHz, DMSO-d) S 9.44 (s, 1H), 9.26 (s, 1H), 8.13-8.06 (m, 2H), 7.48 (dq, J=8.8, 3.0, 2.4 Hz, 4H), 7.18 (s, 1H), 7.08 (d, J=2.2 Hz, 1H), 6.99 (dd, J=8.2, 2.3 Hz, 1H), 6.89 (s, 2H). MS (EI) m/z: 304 (M++H).

Compound 16: 6-(4-(1H-imidazol-1-yl)phenyl)-2-amino-4-(3,4-dihydroxyphenyl)nicotinonitrile. Light yellow solid, Yield: 2.94% (15.7 mg). ¹H NMR (500 MHz, dmso) δ 8.37 (s, 1H), 8.25 (d, J=8.7 Hz, 2H), 7.86-7.83 (m, 1H), 7.78 (d, J=8.7 Hz, 2H), 7.25 (s, 1H), 7.13 (t, J=1.1 Hz, 1H), 7.09 (d, J=2.2 Hz, 1H), 6.99 (dd, J=8.2, 2.2 Hz, 1H), 6.87 (d. J=8.1 Hz, 1H). MS (EI) m/z: 370 (M++H).

Compound 17: 2-amino-4-(3,4-dihydroxyphenyl)-6-(4-(dimethylamino)phenyl)nicotinonitrile. Light yellow solid, Yield: 4.15% (20.8 mg). ¹H NMR (500 MHz, dmso) δ 7.96 (d, J=8.8 Hz, 2H), 6.93 (dd, J=8.2, 2.2 Hz, 1H), 6.85 (d, J=8.2 Hz, 1H), 6.74 (d, J=8.8 Hz, 2H), 6.72 (d, J=2.1 Hz, 1H), 6.69 (s, 1H), 2.96 (s, 6H). MS (EI) m/z: 347 (M++H).

Compound 19: 4-(3,4-dihydroxyphenyl)-6-(4-fluorophenyl)-2-oxo-1,2-dihydropyridine-3-carbonitrile, yellow-brown solid, Yield: 9% (37 mg). ¹H NMR (500 MHz, DMSO-d) δ 12.65 (s, 1H), 9.67 (s, 1H), 9.36 (s, 1H), 7.95 (dd, J=8.6, 5.3 Hz, 2H), 7.38-7.33 (m, 2H), 7.17 (d, J=2.3 Hz, 1H), 7.09 (dd, J=8.2, 2.3 Hz, 1H), 6.89 (d, J=8.3 Hz, 1H), 6.73 (s, 1H). MS (EI) m/z: 323 (M++H).

Compound 20: 4-(3,4-dihydroxyphenyl)-6-(4-fluorophenyl)-2-imino-5-methyl-1,2-dihydropyridine-3-carbonitrile, yellow-brown solid, Yield: 7% (29 mg). 1H NMR (500 MHz, DMSO-d) δ 7.56 (ddd, J=8.9, 5.6, 2.7 Hz, 2H), 7.33-7.22 (m, 2H), 6.84 (d, J=8.0 Hz, 1H), 6.71 (d, J=2.2 Hz, 1H), 6.67 (s, 2H), 6.61 (dd, J=8.1, 2.1 Hz, 1H), 1.85 (s, 3H). MS (EI) m/z: 336 (M++H).

Compound 31: 6-([1,1′-biphenyl]-4-yl)-4-(3,4-dihydroxyphenyl)-2-oxo-2,3-dihydropyridine-3-carbonitrile, yellow-brown solid, 1H NMR (500 MHz, CDCL₃) δ 7.97 (d, J=8.0 Hz, 2H), 7.82 (d, J=8.6 Hz, 1H), 7.75 (d, J=7.1 Hz, 1H), 7.49 (dd, J=8.4, 7.0 Hz, 2H), 7.44-7.37 (m, 1H), 7.17 (d, J=2.3 Hz, 1H), 7.09 (dd, J=8.2, 2.3 Hz, 1H), 6.88 (d, J=8.2 Hz, 1H), 6.76 (s, 1H). MS (EI) m/z: 381 (M++H).

Compound 32: 4-(3,4-dihydroxyphenyl)-6-(4-hydroxyphenyl)-2-oxo-2,3-dihydropyridine-3-carbonitrile, yellow-brown solid. ¹H NMR (500 MHz, CDCL₃) δ 7.93-7.87 (m, 2H), 6.92 (d, J=2.0 Hz, 1H), 6.81 (dd, J=8.7, 2.1 Hz, 2H), 6.76 (dd, =8.1, 2.0 Hz, 1H), 6.71 (s, 1H), 6.61 (d, J=8.1 Hz, 1H). MS (EI) m/z: 321 (M⁺+H).

Compound 33: 5-(2-amino-6-(4-aminophenyl)-3-cyanopyridin-4-yl)-2-hydroxybenzoic acid. ¹H NMR (500 MHz, DMSO) δ 7.95 (s, 1H), 7.84 (d. J=8.7 Hz, 2H), 7.45 (d, J=8.4 Hz, 1H), 6.98 (s, 1H), 6.72 (d, J=10.0 Hz, 1H), 6.63-6.57 (m, 4H), 5.61 (s, 2H). UPC²-MS (ES+) m/z:347 (M++H).

Compound 34: 4-(3,4-dichlorophenyl)-6-(4-fluorophenyl)-2-oxo-1,2-dihydropyridine-3-carbonitrile, yellow solid, Yield: 23% (101 mg). ¹H NMR (500 MHz, DMSO-d) δ 12.92 (s, 1H), 8.05 (d, J=2.1 Hz, 1H), 8.00 (t, J=6.9 Hz, 2H), 7.86 (d, J=8.4 Hz, 1H), 7.74 (dd, J=8.4, 2.2 Hz, 1H), 7.45-7.35 (m, 2H), 6.93 (s, 1H). MS (EI) m/z: 360 (M++H).

Compound 35: 6-(4-fluorophenyl)-2-oxo-4-phenyl-1,2-dihydropyridine-3-carbonitrile, yellow solid, Yield: 14% (50 mg). ¹H NMR (500 MHz, DMSO-d) δ 12.82 (s, 1H), 7.99 (ddd, J=8.5, 5.3, 2.4 Hz, 2H), 7.74 (dt, J=7.5, 2.6 Hz, 2H), 7.57 (q, J=2.8 Hz, 3H), 7.38 (td, J=8.9, 2.4 Hz, 2H), 6.95-6.75 (m, 1H). MS (EI) m/z: 291 (M⁺+H).

Compound 36: 6-(4-fluorophenyl)-2-imino-4-(tetrahydro-2H-pyran-3-yl)-1,2-dihydropyridine-3-carbonitrile, yellow-brown solid, Yield: 2% (8 mg). ¹H NMR (500 MHz, DMSO-d) δ 12.66 (s, 1H), 7.91 (s, 2H), 7.45-7.32 (m, 2H), 6.80 (s, 1H), 3.95-3.78 (m, 2H), 3.45 (td, J=10.9, 3.9 Hz, 1H), 3.09-2.96 (m, 1H), 2.05-1.85 (m, 2H), 1.74-1.57 (m, 2H). MS (EI) m/z: 298 (M⁺+H).

Compound 37: 6(4-fluorophenyl)-4-(5-methylfuran-2-yl)-2-oxo-1,2-dihydropyridine-3-carbonitrile, yellow solid, Yield: 10% (36 mg). ¹H NMR (500 MHz, DMSO-d) δ 12.51 (s, 1H), 7.99-7.83 (m, 2H), 7.61 (d, J=3.5 Hz, 1H), 7.47-7.30 (m, 2H), 6.96 (s, 1H), 6.48 (dd, J=3.6, 1.1 Hz, 1H), 2.42 (s, 3H). MS (EI) m/z: 295 (M⁺+H).

Compound 38: 6-(4-fluorophenyl)-2-imino-4-(naphthalen-2-yl)-1,2-dihydropyridine-3-carbonitrile, yellow-brown oil, Yield: 7% (31 mg). ¹H NMR (500 MHz, DMSO-d) δ 12.85 (s, 1H), 8.33 (d, J=1.8 Hz, 1H), 8.10 (d. J=8.6 Hz, 1H), 8.08-7.98 (m, 4H), 7.83 (dd, J=8.5, 1.9 Hz, 1H), 7.64 (tt, J=6.9, 5.2 Hz, 2H), 7.47-7.30 (m, 2H), 6.98 (s, 1H). MS (EI) m/z: 340 (M⁺+H).

Compound 39: 6-(4-fluorophenyl)-2-hydroxy-4-(2-hydroxyphenyl)nicotinonitrile yellow solid, Yield: 21% (79 mg). ¹H NMR (500 MHz, DMSO-d) δ 8.56 (dd, J=8.1, 1.5 Hz, 1H), 8.41-8.33 (m, 2H), 8.01 (s, 1H), 7.73-7.62 (m, 1H), 7.47-7.33 (m, 4H). MS (EI) m/z: 307 (M⁺+H).

Compound 40: 6-(4-fluorophenyl)-4-(3-hydroxyphenyl)-2-oxo-1,2-dihydropyridine-3-carbonitrile, yellow solid, Yield: 11% (43 mg). ¹H NMR (500 MHz, DMSO-d) δ 12.78 (s, 1H), 9.83 (s, 1H), 7.97 (dd. J=8.6, 5.3 Hz, 2H), 7.36 (q, J=8.3, 7.8 Hz, 3H), 7.12 (dt, J=7.7, 1.3 Hz, 1H), 7.08 (t, J=2.1 Hz, 1H), 6.95 (ddd, J=8.2, 2.5, 0.9 Hz, 1H), 6.82-6.78 (m, 1H). MS (EI) m/z: 307 (M++H).

Compound 41: 6(4-fluorophenyl)-4-(4-hydroxyphenyl)-2-oxo-1,2-dihydropyridine-3-carbonitrile, yellow solid, Yield: 9% (34 mg). ¹H NMR (500 MHz, DMSO-d) δ 12.65 (s, 1H), 10.12 (s, 1H), 7.99-7.92 (m, 2H), 7.67-7.60 (m, 2H), 7.41-7.33 (m, 2H), 6.96-6.89 (m, 2H), 6.77 (s, 1H). MS (EI) m/z: 307 (M++H).

Compound 42: 6(4-fluorophenyl)-4-(4-hydroxy-3-methoxyphenyl)-2-oxo-1,2-dihydropyridine-3-carbonitrile. yellow solid, Yield: 10% (42 mg). ¹H NMR (500 MHz, DMSO-d) δ 12.65 (s, 1H), 9.72 (s, 1H), 8.00-7.92 (m, 2H), 7.42-7.32 (m, 3H), 7.24 (dd.J=8.2, 2.2 Hz, 1H), 6.93 (d, J=8.2 Hz, 1H), 6.83 (s, 1H), 3.86 (s, 3H). MS (EI) m/z: 337 (M++H).

Compound 43: 6-(4-fluorophenyl)-2-imino-4-phenyl-1,2-dihydropyridine-3-carbonitrile. yellow-brown solid, Yield: 5% (19 mg). ¹H NMR (500 MHz, DMSO-d) δ 8.24-8.15 (m, 2H), 7.70-7.62 (m, 2H), 7.59-7.43 (m, 3H), 7.34-7.21 (m, 3H), 7.01 (s, 2H). MS (EI) m/z: 290 (M++H).

Compound 44: 6-(4-fluorophenyl)-2-imino-4-(5-methylfuran-2-yl)-1,2-dihydropyridine-3-carbonitrile. yellow-brown solid, Yield: 11% (39 mg). ¹H NMR (500 MHz, DMSO-d) δ 8.18-8.10 (m, 2H), 7.44 (s, 1H), 7.41 (d, J=3.4 Hz, 1H), 7.36-7.27 (m, 2H), 6.93 (s, 2H), 6.39 (dd, J=3.5, 1.1 Hz, 1H), 2.40 (s, 3H). MS (EI) m/z: 294(M++H).

Compound 45: 6-(4-fluorophenyl)-2-imino-4-(5-methylthiophen-2-yl)-1,2-dihydropyridine-3-carbonitrile. yellow-brown solid, Yield: 6% (22 mg). ¹H NMR (500 MHz, CD3OD) δ 7.74-7.44 (m, 3H), 7.36 (d, J=3.7 Hz, 1H), 7.20 (dt, J=41.7, 8.4 Hz, 2H), 6.88-6.62 (m, 2H), 2.42 (d, J=11.9 Hz, 3H). MS (EI) m/z: 310 (M++H).

Compound 46: 6-(4-fluorophenyl)-4-(4-hydroxyphenyl)-2-oxo-1,2-dihydropyridine-3-carbonitrile. yellow solid, Yield: 9% (34 mg). ¹H NMR (500 MHz, DMSO-d) δ 12.65 (s, 1H), 10.12 (s, 1H), 7.99-7.92 (m, 2H), 7.67-7.60 (m, 2H), 7.41-7.33 (m, 2H), 6.96-6.89 (m, 2H), 6.77 (s, 1H). MS (EI) m/z: 307 (M++H).

Compound 47: 2-amino-6-(4-fluorophenyl)-4-(2-hydroxyphenyl)nicotinonitrile. yellow-brown solid. Yield: 8% (31 mg). ¹H NMR (500 MHz, DMSO-d) δ 8.57 (dd, J=8.1, 1.5 Hz, 1H), 8.42-8.33 (m, 2H), 8.01 (s, 1H), 7.66 (ddd, J=8.4, 7.3, 1.5 Hz, 1H), 7.47-7.32 (m, 4H). MS (EI) m/z: 306 (M++H).

Compound 48: 6-(4-fluorophenyl)-4-(4-hydroxy-3-methoxyphenyl)-2-imino-1,2-dihydropyridine-3-carbonitrile. yellow-brown solid, Yield: 9% (37 mg). ¹H NMR (500 MHz, DMSO-d) δ 9.52 (s, 1H), 8.25-8.14 (m, 2H), 7.36-7.29 (m, 2H), 7.27 (d, J=1.9 Hz, 2H), 7.22-7.10 (m, 1H), 6.98-6.85 (m, 4H), 3.86 (s, 3H). MS (EI) m/z: 336 (M++H).

Compound 49: 6-(4-fluorophenyl)-4-(3-hydroxy-4-methoxyphenyl)-2-imino-1,2-dihydropyridine-3-carbonitrile. yellow-brown solid, Yield: 7% (28 mg). ¹H NMR (500 MHz, DMSO-d) δ 8.21-8.15 (m, 2H), 7.36-7.29 (m, 2H), 7.21 (s, 1H), 7.18-7.10 (m, 2H), 7.09-7.04 (m, 1H), 6.96 (s, 2H), 3.85 (s, 3H). MS (EI) W/z: 336 (M++H).

Compound 50: 6′-(4-fluorophenyl)-2′-imino-1′,2′-dihydro-[3,4′-bipyridine]-3′-carbonitrile. yellow-brown solid. Yield: 6% (21 mg). ¹H NMR (500 MHz, DMSO-d) δ 8.88 (s, 1H), 8.79-8.70 (m, 1H), 8.33-8.20 (m, 2H), 8.13 (dt, J=7.9, 2.0 Hz, 1H), 7.60 (dd. J=7.9, 4.8 Hz, 1H), 7.39 (d, J=3.8 Hz, 1H), 7.37-7.31 (m, 2H), 7.15 (s, 2H). MS (EI) m/z: 291 (M++H).

Compound 51: 5-(3-cyano-6-(4-fluorophenyl)-2-imino-1,2-dihydropyridin-4-yl)-2-hydroxybenzoic acid, yellow-brown solid, Yield: 2% (8.8 mg). ¹H NMR (500 MHz, DMSO-d) δ 8.43-8.35 (m, 2H), 8.14 (s, 1H), 8.22-8.03 (m, 1H), 7.39-7.30 (m, 3H), 6.99 (dd, J=122.1, 8.5 Hz, 1H). MS (EI) m/z: 350 (M++H).

Compound 52: 4-(3-bromo-4-hydroxyphenyl)-6(4-fluorophenyl)-2-imino-1,2-dihydropyridine-3-carbonitrile. yellow-brown solid, Yield: 10% (47 mg). ¹H NMR (500 MHz, DMSO-d) δ 10.82 (s, 1H), 8.24-8.16 (m, 2H), 7.85 (d, J=2.3 Hz, 1H), 7.55 (dd, J=8.4, 2.3 Hz, 1H), 7.31 (t, J=8.8 Hz, 2H), 7.10 (d, J=8.4 Hz, 1H), 7.00 (s, 2H). MS (EI) m/z: 385 (M++H).

Compound 53: 6(4-fluorophenyl)-4-(4-hydroxy-3-iodophenyl)-2-imino-1,2-dihydropyridine-3-carbonitrile. yellow-brown solid, Yield: 8% (43 mg). ¹H NMR (500 MHz, DMSO-d) δ 10.88 (s, 1H), 9.98 (d, J=4.4 Hz, 1H), 8.19 (ddd, J=10.9, 6.6, 3.7 Hz, 2H), 8.01 (d, J=2.3 Hz, 1H), 7.59-7.51 (m, 1H), 7.29 (t, J=8.8 Hz, 3H), 7.03 (d, J=8.4 Hz, 1H), 6.98 (s, 2H). MS (EI) m/z: 432 (M++H).

Compound 54/1A: 6-(bicyclo[1.1.1]pentan-1-yl)-4-(3,4-dihydroxyphenyl)-2-imino-2,3-dihydropyridine-3-carbonitrile. Light yellow solid, Yield: 7.4% (10.0 mg). ¹H NMR (500 MHz, CDCL₃) δ 7.91 (s, 2H), 7.54 (dd, J=8.6, 1.7 Hz, 2H) 7.31 (dd, J=2.0, 0.3 Hz, 1H), 7.02 (dd, J=8.84, 0.5 Hz, 1H), 7.05 (sept, J=3.2 Hz, 1H), 2.5-1.7 (m, 6H). MS (EI) m/z: 294 (M++H).

Compound 55/2A: 2-amino-6-cyclopropyl-4-(3,4-dihydroxyphenyl) nicotinonitrile. Light yellow solid, Yield: 33.1% (128 mg), 1H NMR (500 MHz, dmso) S 6.95 (d, J=2.1 Hz, 1H), 6.85 (dd, J=8.1, 2.1 Hz, 1H), 6.82 (d, J=8.2 Hz, 1H), 6.55 (s, 1H), 1.98 (p, J=6.4 Hz, 1H), 0.93 (s, 2H), 0.92 (s, 2H). MS (EI) m/z: 268 (M++H).

Compound 56/3A: 2-amino-6-cyclobutyl-4-(3,4-dihydroxyphenyl) nicotinonitrile. Light yellow solid, Yield: 7.44% (30.3 mg). ¹H NMR (500 MHz, dmso) δ 6.95 (d, J=2.1 Hz, 1H), 6.85 (d, J=2.2 Hz, 1H), 6.83 (s, 1H), 6.46 (s, 1H), 3.49 (p, J=8.7 Hz, 1H), 2.24-2.16 (m, 4H), 2.01-1.90 (m, 2H). MS (EI) m/z: 282 (M++H).

Reagents. Tag-free HEK-293 expressed IL-4 for HEK-Blue IL-4/IL-13, THP-1, and Ramos inhibition assays purchased from Acros Biosystems (IL4-H4218). Tag-free mouse IL-4 (574302) for Raw 264.7 macrophage Inhibition assay was purchased from BioLegend.

Avitag IL-4 for surface plasmon resonance was purchased from Acros Biosystems (IL4-H82 E0). Biotin-Streptavidin Sensors Kit for surface plasmon resonance was purchased from Cytiva (BR100531). Blasticidin, Zeocin, Normocin, and Quanti-Blue were purchased from Invivogen. pSTAT6 (690102). STAT6(690101) antibodies for THP-1 and Ramos Inhibition assays were purchased from Biolegend. pSTAT6 antibody (PI700247) for the RAW Macrophage Inhibition assay was purchased from Fisher Scientific. All other chemicals and reagents were purchased from Fisher Scientific and Sigma Aldrich.

HEK-Blue IL-4/IL-13 Inhibition assay. HEK-Blue IL-4/IL-13 reporter cell lines were purchased from Invivogen and cultured in DMEM with 10% Heat Inactivated Fetal Bovine Serum (FBS), 100 U/ml-100 μg/ml Pen-Strep, 10 μg/ml blasticidin, 100 μg/ml Normocin and 100 μg/ml Zeocin. Cells were tested for secreted embryonic alkaline phosphatase (SEAP) production induced by IL-4 alone prior to small molecule screening. It was determined to be 0.3 ng/ml, 0.1 ng/ml was used as the final well concentration of IL-4 (EC₂₅ for IL-4 was chosen for screening small molecules to determine their inhibitory activity)⁴⁸.

22.5 μl of small molecule in DMEM 4% DMSO was added to 22.5 μl of 1 ng/ml IL-4 in DMEM in a non-binding 96-well reaction plate for 30 minutes at room temperature. The reaction plate was further incubated for 30-60 min at 37° C. HEK-Blue IL-4/IL-13 reporter cell line was used between passages 11-17. Cells were washed with 1×PBS, trypsinized using 0.25% Trypsin-EDTA (1×) for 2 minutes, and centrifuged for 5 minutes at 250×g. Cells were resuspended in DMEM 10% FBS and 100 U/ml-100 μg·ml Pen-Strep at a concentration of 3.125 E5/ml, 160 μl of cell suspension was added to each well of a 96-well plate, avoiding edges, and incubated for 30 minutes in a 37° C./5% CO2 incubator. Lastly, 40 μl of IL-4 with small molecule/vehicle solution was added to the cells preincubated in a 96-well plate. Measurements were done in triplicate. The plate was incubated for ˜22-24 hours at 37° C./5% CO2. Quanti-Blue dye was prepared as per the manufacturer's recommendation and 160 μl was dispensed in each well of a 96-well plate (avoid edges to avoid evaporation), 40 μl of cell supernatant was added to the same plate and incubated for 3 hours at 37° C./5% CO2. Optical density was then measured at 650 nm using Molecular Devices SpectraMax M5 Microplate reader. Data was analyzed and plotted using GraphPad Prism 9.2.0.

HEK-Blue IL-4/IL-13 reporter cell lines were also used to screen Nico-52 analogs against IL-13 similarly. Prior to screening. IL-13 induced SEAP production was quantified with IL-13 concentrations ranging from 0.2-100 μg/ml with 0.04% DMSO in DMEM (final well concentration), 8 ng/ml of IL-13 was incubated with increasing concentrations of Nico-52.18 and Nico-52.19 with 4% DMSO in DMEM for 30 minutes at room temperature and 30-60 min at 37° C. The solution was added to preincubated cells in a 96-well culture plate. The supernatant was then incubated with Quanti-Blue dye for 3 hours and absorbance was measured at 650 nm. Data was analyzed and plotted using GraphPad Prism 9.2.0.

TABLE 4
Control data for HEK-Blue IL-4/IL-13 inhibition experiments.

| Condition | Abs650 |
|---|---|
| Cells alone | 0.05825 ± 0.00035 |
| Cells with vehicle | 0.05753 ± 0.000791 |
| Cells with IL-4 and vehicle | 0.23874 ± 0.037224 |
| Cells with 10 μM Compound 14 only | 0.05948 ± 0.000642 |
| Cells with 10 μM Compound 15 only | 0.06345 ± 0.001036 |

### Differential Scanning Fluorimetry

Certain Nico-52 analogs were tested for selectivity across cytokines IL-2, IL-7, IL-21 and IL-13, 1 μg of cytokine in PBS was added to each well in 384-well plates followed by the addition of 8× Spiro-Orange dye proceeded with PBS, DMSO. or analogs. Analogs were screened at a final concentration of 2 μM small molecule in 0.1% DMSO. DSF was performed using BioRad CFX384. Melting temperature shifts were calculated by subtracting the nadir of the melting curve of cytokine with the vehicle from the nadir of the melting curve of the cytokine with analogs. A cut-off of >0.5° C. was used for binding determination. Plots were made and analyzed using GraphPad Prism 9.2.0.

### THP-1 STAT-6 Phosphorylation Assay

THP-1 cells were received as a gift from the laboratory of Dr. Mark Grinstaff and maintained in RMPI-1640 with 100% heat-inactivated Fetal Bovine Serum and 50 μM beta-mercaptoethanol. Cells used for assays were between passages 6 and 10, 10 ng/mL IL-4 was preincubated with experimental sample (vehicle or analogs) for 30 minutes at 37° C. 1 mL of each solution was then added to a pellet of 2 E6 THP-1 cells and pipetted up and down. THP-1 cells were incubated for 30 minutes in the incubator at 37 C/5% CO₂. Cells were then spun down and lysed with 200 μL ice-cold RIPA buffer complete with phosphatase and protease inhibitors. Cell lysates were incubated on ice for 30 minutes, spun down and the supernatants were stored at −20° C. until western blot analysis. Undiluted cell lysates were run on SDS-PAGE and transferred to Immunoblot PVDF membrane using Biorad equipment. Membranes were incubated with 1:1000 primary antibody overnight at 4° C. Membranes were then incubated with 1:50,000 dilution secondary for 1 hour at room temperature. Membranes were then exposed to Femto ECL for 5 minutes and imaged using a Bio-Rad Gel Doc. The brightness/contrast was adjusted in FIJI and the area under the curve of each lane was quantified. The phosphorylated STAT-6 to STAT-6 ratio was calculated and normalized to the amount of total STAT-6. The data was plotted and analyzed using GraphPad Prism 9.2.0′.

### Ramos STAT-6 Phosphorylation Assay

Ramos cells were purchased from ATCC and maintained in RMPI-1640 with 10% heat-inactivated Fetal Bovine Serum. Cells used for assays were between passages 4 and 8. Cell lysate for IL-4 or IL-4 and Nico-52/analogs treated cells were generated similarly to the THP-1 Inhibition assay. Undiluted cell lysates were run SDS-PAGE and transferred to Immunoblot PVDF membrane using Biorad equipment. Membranes were incubated with 1:1000 primary antibody overnight at 4° C. Membranes were then incubated with 1:50,000 dilution secondary for 1 hour at room temperature. Membranes were then exposed to Femto ECL for 5 minutes and imaged using a Bio-Rad Gel Doc. The brightness/contrast was adjusted in FIJI and the area under the curve of each lane was quantified. The phosphorylated STAT-6 to STAT-6 ratio was calculated and normalized to the amount of total STAT-6. The data was plotted and analyzed using GraphPad Prism 9.2.0.

### Raw 264.7 STAT-6 Phosphorylation Assay

RAW 264.7 cells were purchased from ATCC and maintained in DMEM with 10% heat-inactivated Fetal Bovine Serum. Cells used for assays were between passages 3 and 5. Cell lysates for IL-4 or IL-4 and Nico-52 treated cells were generated similarly to the THP-1 Inhibition assay, but murine IL-4 was used at a concentration of 5 ng/ml. The undiluted cell lysates were run SDS-PAGE followed by transfer to Immunoblot PVDF membrane using Biorad equipment. Membranes were incubated with 1:1000 primary antibody overnight at 4° C. Membranes were then incubated with 1:50,000 dilution secondary for 1 hour at room temperature. Membranes were then exposed to Femto ECL for 5 minutes and imaged using a Bio-Rad Gel Doc. The brightness/contrast was adjusted in FIJI and the area under the curve of each lane was quantified. The phosphorylated STAT-6 to STAT-6 ratio was calculated and normalized to the amount of total STAT-6. The data was plotted and analyzed using GraphPad Prism 8.

THP-1 pSTAT-6 Immunofluorescence

IL-4 and analogs or vehicle (DMEM with 2% DMSO) were preincubated for 2 hours at 37° C. After the incubation, IL-4 or IL-4/analogs were incubated with THP-1 cells for 30 min at 37° C./5% CO₂. Incubated cells were centrifuged for 5 min at 250×g. After aspirating the supernatant, cells were resuspended in 1×PBS and plated into a MatTek dish coated with Cell Tak adhesive for 30 minutes at room temperature. Cells were fixed with 4% Paraformaldehyde for 1 hour at room temperature. Cells were rinsed twice with 1×PBS and then permeabilized with 0.5% Triton-X for 1 hour at room temperature. Cells were rinsed again with 1×PBS, and blocked for an hour in superblock with 0.05% Tween-20, rinsed twice, and then incubated with anti-pSTAT-6 1:100 diluted in superblock 0.05% Tween-20 overnight at 4° C. (parafilmed to avoid evaporation). Cells were rinsed thrice with 1×PBS and then incubated with Mouse anti-rabbit IgG(H+L) Secondary Antibody-Alexa Fluor 647 for 1 hour at room temperature in the dark. Cells were rinsed thrice with 1×PBS and then a dilution of 1:100 Hoechst 33342 dye was added to the cells for 15-20 minutes at room temperature in the dark. Cells were rinsed twice with 1×PBS and left in 1 ml of 1×PBS. Confocal Imaging was done using an Olympus FV10i and images were processed using FIJI.

### Surface Plasmon Resonance

Surface plasmon resonance was performed on a Biacore S200. Series S Sensor chips were purchased from Cytiva. The running buffer was 1×PBS pH 7.40 with 2% DMSO and 0.1% Tween-20 and the flow rate was kept at 50 μL/min. The SA chip was first activated with 120 μL of 1 M NaCl in 50 mM NaOH followed by a wash of 50% Isopropanol in 1 M NaCl and 50 mM NaOH, 200 μl of a 1 mg/ml solution of Avi-tagged IL-4 in DMSO-free buffer was used to immobilize 7073 RU on the chip surface using the BiacoreS200 software, 100 μl of 50 mM Biocytin was used to block all biotin slides on the sensor chip with manual setting. The system was then primed with running buffer for 7 minutes. The sensor's surface was verified by using Nico-52 at 1.25 μM dissolved in running buffer prior to doing further runs. Analog 15 was diluted in the running buffer and injected for 300 seconds cycles, in an ascending order. The dissociation phase was 600 seconds long. Sensorgram figures were created by exporting the raw data and plotting in Graphpad Prism 9.2.0⁴⁸.

Hepatic Microsomes, 0.5 mg/mL Mouse (CD-1) liver microsomes purchased from GIBCO MSMCPL were incubated with 50 μM of each compound at 37° C./5% CO₂ with 1 mM NADPH for 0 (control) or 60 minutes, and 60 minutes without NADPH (NADPH independent metabolism) before quenching with equal volume of acetonitrile and analyzed by UPLC-MS, quantified by AUC of total absorbance with stability reported as % remaining compared to control.

### Plasma Stability

Compounds at 50 μM were incubated for 0 (control) or 180 minutes in plasma from C57BL/6 mouse or pooled human both purchased from Valley Biomedical at 37° C./5% CO₂ before quenching with equal volume of acetonitrile and analyzed by UPLC-MS, quantified by AUC of total absorbance with plasma stability reported as % remaining compared to control.

### Pampa Membrane Permeability

PVDF membranes purchased from Bioassay Systems PAMPA-096 were coated with 5 μL of 4% Phosphatidylcholine (Lecithin, MP Biomedicals 0210214780) in dodecane. Donor chambers were filled with 300 μL of the compound at 100 μM, acceptor chambers filled with equal volume of PBS, and then the plate was incubated for 18 hours at 37° C./5% CO2 before quenching with equal volume of acetonitrile and analyzed by UPLC-MS. quantified by AUC of total absorbance. Permeability was calculated by Pᵥ=C×ln (1−OD_{A}/OD_{E}) cm/s, where OD_{A} is the absorbance of acceptor solution minus blank, and OD_{E} the absorbance of the equilibrium standard minus blank, and C=7.72×10⁻⁶ using an 18-hour incubation time.

In vitro Cytotoxicity and Growth Inhibition

CellTiter-Glo 2.0 purchased from Promega G9242) was used to measure cell viability with the Victor-3 plate reader (PerkinElmer) after 24 hours in concentrations of compound up to 25 μM. Vehicle was used as a negative control for both cytotoxicity and growth inhibition, methanol was used as a positive control for cytotoxicity, and 1% FBS was used as a positive control for growth inhibition.

### REFERENCES

(1) Iwaszko, M.; Bialy, S.; Bogunia-Kubik, K. Significance of Interleukin (IL)-4 and IL-13 in Inflammatory Arthritis. Cells 2021, 10 (11), 3000. https://doi.org/10.3390/cells10113000.
(2) Bagnasco, D., Ferrando, M.; Varricchi, G.; Passalacqua, G., Canonica, G. W. A Critical Evaluation of Anti-IL-13 and Anti-IL-4 Strategies in Severe Asthma. Int Arch Allergy immunol 2016, 170 (2), 122-131. https://doi.org/10.1159/000447692.
(3) Oh, C. K.; Geba, G. P.; Molfino, N. Investigational Thera-peutics Targeting the IL-4/IL-13/STAT-6 Pathway for the Treat-ment of Asthma. European Respiratory Review 2010, 19 (115), 46-54. https://doi.org/10.1183/09059180.00007609.
(4) DeNardo. D. G.: Barreto, J. B.; Andreu. P.; Vasquez. L.; Tawfik. D.; Kolhatkar, N.; Coussens, L. M. CD4+ T Cells Regulate Pulmonary Metastasis of Mammary Carcinomas by Enhancing Protumor Properties of Macrophages. Cancer Cell 2009, 16 (2), 91-102. https://doi.org/10.1016/j.ccr. 2009.06.018.
(5) Gocheva. V.; Wang, H. W.; Gadea, B. B.; Shree. T.; Hunter, K. E.; Garfall, A. L.; Berman, T.; Joyce, J. A. IL-4 Induces Cathepsin Protease Activity in Tumor-Associated Macrophages to Promote Cancer Growth and Invasion. Genes Dev. 2010, 24 (3), 241-255. https://doi.org/10.1101/gad. 1874010.
(6) Li, B. H.; Yang, X. Z.; Li, P. D.; Yuan, Q.; Liu, X. H.; Yuan, J.; Zhang, W. J. IL-4/Stat6 Activities Correlate with Apoptosis and Metastasis in Colon Cancer Cells. Biochemical and Biophysical Research Communications 2008, 369 (2), 554-560. https://doi.org/10.1016/j.bbrc. 2008.02.052.
(7) Suzuki, A.; Leland, P.; Joshi, B. H.; Puri, R. K. Targeting of IL-4 and IL-13 Receptors for Cancer Therapy. Cytokine 2015, 75 (1), 79-88. https://doi.org/10.1016/j.cyto. 2015.05.026.
(8) Wills-Karp, M.; Finkelman, F. D. Untangling the Complex Web of IL-4- and IL-13-Mediated Signaling Pathways. Sci. Signal. 2008, 1 (51). https://doi.org/10.1126/scisignal. 1.51.pe55.
(9) Castro, M.; Corren, J.; Pavord. I. D.; Maspero, J.; Wenzel, S.; Rabe, K. F.; Busse, W. W.; Ford, L.; Sher, L.; FitzGerald, J. M.; Katelaris, C.; Tohda, Y.; Zhang, B.; Staudinger, H.; Pirozzi, G.; Amin, N.; Ruddy, M.; Akinlade, B.; Khan, A.; Chao, J.; Martincova, R.; Gra-ham. N. M. H.; Hamilton, J. D.; Swanson, B. N.; Stahl, N.; Yancopou-los, G. D.; Teper, A. Dupilumab Efficacy and Safety in Moderate-to-Severe Uncontrolled Asthma. N Engl J Med 2018, 378 (26), 2486-2496. https://doi.org/10.1056/NEJMoa1804092.
(10) Galletti, C.; Barbieri, M. A.; Ciodaro, F.; Freni, F.; Galletti, F.; Spina. E.; Galletti, B. Effectiveness and Safety Profile of Dupi-lumab in Chronic Rhinosinusitis with Nasal Polyps. Real-Life Data in Tertiary Care. Pharmaceuticals 2023, 16 (4), 630. https://doi.org/10.3390/ph16040630.
(11) Grey, A.; Katelaris, C. H. Dupilumab in the Treatment of Asthma. Immunotherapy 2019, 11 (10), 859-872. https://doi.org/10.2217/imt-2019-0008.
(12) Maier, B.; Leader, A. M.; Chen, S. T.; Tung, N.; Chang, C.; LeBerichel, J.; Chudnovskiy, A.; Maskey, S.; Walker, L.; Finnigan, J. P.; Kirkling, M. E.; Reizis, B.; Ghosh, S.; D'Amore, N. R.; Bhardwaj, N.; Rothlin, C. V.; Wolf, A.; Flores, R., Marron, T.; Rahman, A. H.; Kenigsberg, E.; Brown, B. D.; Merad, M. A Conserved Dendritic-Cell Regulatory Program Limits Antitumour Immunity. Nature 2020, 580 (7802), 257-262. https://doi. org/10.1038/s41586-020-2134-y.
(13) Fitzgerald, B. G.: Marron, T. U.; Hall, N.; O'Grady, D.; La-Marche, N.; Hennequin, C.; Hegde, S.; Maier, B.; Le Berichel, J.; Chadda, U.; Beasley, M. B.; Yankelevitz, D. F.; Gomez, J. E.; Doroshow, D. B.; Veluswamy, R.; Rohs, N. C.; Rolfo, C. D.; Hirsch, F. R.; Merad, M. A Phase 1b/2 Trial of Dupilumab given in Conjunc-tion with PD-(L)1 Blockade in the Treatment of Re-lapsed/Refractory Metastatic NSCLC. JCO 2022, 40 (16_suppl), TPS9139-TPS9139. https://doi.org/10.1200/JCO. 2022.40.16_suppl.TPS9139.
(14) Castelli, M. S.; McGonigle, P.; Hornby, P. J. The Pharma-cology and Therapeutic Applications of Monoclonal Antibodies. Pharmacology Res & Perspec 2019, 7 (6), e00535. https://doi.org/10.1002/prp2.535.
(15) Silberstein, S.; Lenz, R.; Xu, C. Therapeutic Monoclonal Antibodies: What Headache Specialists Need to Know. Headache 2015, 55 (8), 1171-1182. https://doi.org/10.11/head. 12642.
(16) Deng, R; Jin, F.; Prabhu, S.; Iyer, S. Monoclonal Antibod-ies: What Are the Pharmacokinetic and Pharmacodynamic Con-siderations for Drug Development? Expert Opinion on Drug Me-tabolism & Toxicology 2012, 8 (2), 141-160. https://doi.org/10.1517/17425255.2012.643868.
(17) Singh, A. P.; Shin, Y. G.; Shah, D. K. Application of Phar-macokinetic-Pharmacodynamic Modeling and Simulation for An-tibody-Drug Conjugate Development. Pharm Res 2015, 32 (11), 3508-3525. https://doi.org/10.1007/s11095-015-1626-1.
(18) Dostalek, M.; Gardner. I.; Gurbaxani, B. M.; Rose, R. H.. Chetty, M. Pharmacokinetics, Pharmacodynamics and Physiologi-cally-Based Pharmacokinetic Modelling of Monoclonal Antibodies. Clin Pharmacokinet 2013, 52 (2), 83-124. https://doi.org/10.1007/s40262-012-0027-4.
(19) Mahalingaiah, P. K.; Ciurlionis, R.; Durbin, K. R.; Yeager, R. L.; Philip, B. K.; Bawa, B.; Mantena. S. R.; Enright, B. P.; Liguori, M. J.; Van Vleet, T. R. Potential Mechanisms of Target-Independent Uptake and Toxicity of Antibody-Drug Conjugates. Pharmacology & Therapeutics 2019, 200, 110-125. https://doi.org/10.1016/j.pharmthera. 2019.04.008.
(20) Catapano, A. L.; Papadopoulos, N. The Safety of Thera-peutic Monoclonal Antibodies: Implications for Cardiovascular Disease and Targeting the PCSK9 Pathway. Atherosclerosis 2013, 228 (1), 18-28. https://doi.org/10.1016/j.atherosclerosis. 2013.01.044.
(21) Giblin, K. A.; Basili, D.; Afzal, A. M.; Rosenbrier-Ribeiro, L.; Greene, N.; Barrett, I.; Hughes, S. J.; Bender, A. New Associations between Drug-Induced Adverse Events in Animal Models and Humans Reveal Novel Candidate Safety Targets. Chem. Res. Toxicol. 2021, 34 (2), 438-451. https://doi.org/10.1021/acs.chemrestox. 0c00311.
(22) Gómez-Mantilla, J. D.; Trocóniz., I. F.; Parra-Guillén, Z.; Garrido, M. J. Review on Modeling Anti-Antibody Responses to Monoclonal Antibodies. J Pharmacokinet Pharmacodyn 2014, 41 (5), 523-536. https://doi.org/10.1007/s10928-014-9367-z.
(23) Büttel, I. C.; Chamberlain, P.; Chowers, Y.; Ehmann, F.; Greinacher, A.; Jefferis, R.; Kramer, D.; Kropshofer, H.; Lloyd, P.; Lubiniecki, A.; Krause, R.; Mire-Sluis, A.; Platts-Mills, T.; Ragheb, J. A.; Reipert, B. M.; Schellekens, H.; Seitz, R.; Stas, P.; Subramanyam, M.; Thorpe, R.; Trouvin, J. H.; Weise, M.; Windisch, J.; Schneider, C. K. Taking Immunogenicity Assessment of Therapeutic Proteins to the next Level. Biologicals 2011, 39 (2), 100-109. https://doi.org/10.1016/j.biologicals. 2011.01.006.
(24) Nada, H.; Sivaraman, A.; Lu, Q.; Min, K.; Kim, S.; Goo, J. I.; Choi, Y.; Lee, K. Perspective for Discovery of Small Molecule IL-6 Inhibitors through Study of Structure-Activity Relationships and Molecular Docking. J. Med. Chem. 2023, 66 (7), 4417-4433. https://doi.org/10.1021/acs.jmedchem. 2c01957.
(25) Gilardi, D.; Gabbiadini, R.; Allocca, M.; Correale, C.; Fiori-no. G.; Furfaro, F.; Zilli, A.; Peyrin-Biroulet, L.; Danese, S. P K, P D, and Interactions: The New Scenario with JAK Inhibitors and SIP Receptor Modulators, Two Classes of Small Molecule Drugs, in IBD. Expert Review of Gastroenterology & Hepatology 2020, 14 (9), 797-806. https://doi.org/10.1080/17474124.2020.1785868.
(26) Diao, L.; Meibohm, B. Pharmacokinetics and Pharmaco-kinetic-Pharmacodynamic Correlations of Therapeutic Peptides. Clin Pharmacokinet 2013, 52 (10), 855-868. https://doi.org/10.1007/s40262-013-0079-0.
(27) Di, L. Strategic Approaches to Optimizing Peptide ADME Properties. AAPS J 2015, 17 (1), 134-143. https://doi.org/10.1208/s12248-014-%87-3.
(28) Makurvet, F. D. Biologics vs. Small Molecules: Drug Costs and Patient Access. Medicine in Drug Discovery 2021, 9, 100075. https://doi.org/10.1016/j.medidd. 2020.100075.
(29) Zheng. J.; Chen, D.; Xu, J.; Ding, X.; Wu. Y.; Shen, H. C.; Tan, X. Small Molecule Approaches to Treat Autoimmune and Inflammatory Diseases (Part III): Targeting Cytokines and Cyto-kine Receptor Complexes. Bioorganic & Medicinal Chemistry Let-ters 2021, 48, 128229. https://doi.org/10.1016/j.bmcl. 2021.128229.
(30) Sanford, M.; McKeage, K. Secukinumab; First Global Approval. Drugs 2015, 75 (3), 329-338. https://doi.org/10.1007/s40265-015-0359-0.
(31) Zhang, B.; Dömling, A. Small Molecule Modulators of IL-17A/IL-17R A: A Patent Review (2013-2021). Expert Opinion on Therapeutic Patents 2022, 32 (11), 1161-1173. https://doi.org/10.1080/13543776.2022.2143264.
(32) Liu, S.; Dakin, L. A.; Xing, L.; Withka, J. M.; Sahasrabudhe, P. V.; Li, W.; Banker, M. E.; Balbo, P.; Shanker, S.; Chrunvk, B. A.; Guo, Z.; Chen, J. M.; Young, J. A.; Bai. G.; Starr, J. T.; Wright, S. W.; Bussenius, J.; Tan, S.; Gopalsamy, A.; Lefler, B. A.; Vincent, F.; Jones, L. H.; Xu, H.; Hoth, L. R.; Geoghegan, K. F.; Qiu, X.; Bunnage, M. E.; Thorarensen, A. Binding Site Elucidation and Structure Guided Design of Macrocyclic IL-17A Antagonists. Sci Rep 2016, 6 (1), 30859. https://doi.org/10.1038/srep30859.
(33) Vanamee, E. S.; Faustman, D. L. Structural Principles of Tumor Necrosis Factor Superfamily Signaling. Sci. Signal. 2018, 11 (511), eaao4910. https://doi.org/10.1126/scisignal.aao4910.
(34) Atzeni, F.; Nucera, V.; Gerratana. E.; Cirillo, M.; Marino, F.; Miceli, G., Sangari, D.; Boccassini, L.; Masala, I. F. Concerns about the Safety of Anti-TNF Agents When Treating Rheumatic Diseases. Expert Opinion on Drug Safety 2020, 19 (6), 695-705. https://doi.org/10.1080/14740338.2020.1763299.
(35) He, M. M.; Smith, A. S.; Oslob, J. D.; Flanagan, W. M.; Braisted, A. C.; Whitty, A.; Cancilla. M. T.; Wang, J.; Lugovskoy, A. A.; Yobum, J. C.; Fung, A. D.; Farrington. G.; Eldredge, J. K.; Day, E. S.; Cruz, L. A.; Cachero, T. G.; Miller, S. K.; Friedman, J. E.; Choong, I. C.; Cunningham, B. C. Small-Molecule Inhibition of TNF-α. Science 2005, 310 (5750), 1022-1025. https://doi.org/10.1126/science. 1116304.
(36) Sun, W., Wu, Y.; Zheng, M.; Yang, Y.; Liu, Y.; Wu, C.; Zhou. Y.; Zhang, Y.; Chen, L.; Li, H. Discovery of an Orally Active Small-Molecule Tumor Necrosis Factor-α Inhibitor. J. Med. Chem. 2020, 63 (15), 8146-8156. https://doi.org/10.1021/acs.jmedchem. 0c00377.
(37) Connelly. C. M.; Abulwerdi, F. A.; Schneekloth, J. S. Dis-covery of RNA Binding Small Molecules Using Small Molecule Microarrays. Methods Mol Biol 2017, 1518, 157-175. https://doi.org/10.1007/978-1-4939-6584-7_11.
(38) Xiao, H. Y.; Li, N.; Duan, J. J. W.; Jiang, B.; Lu, Z.; Ngu, K.; Tino, J.; Kopcho, L. M., Lu, H.; Chen, J.; Tebben, A. J.; Sheriff, S.; Chang, C. Y.; Yanchunas, J.; Calambur, D.; Gao, M.; Shuster, D. J.; Susulic, V.; Xie, J. H.; Guarino, V. R.; Wu, D. R.; Gregor, K. R.; Gold-stine, C. B.; Hynes, J.; Macor, J. E.; Salter-Cid, L.; Burke, J. R.; Shaw, P. J.; Dhar, T. G. M. Biologic-like In Vivo Efficacy with Small Mole-cule Inhibitors of TNFα Identified Using Scaffold Hopping and Structure-Based Drug Design Approaches. J. Med. Chem. 2020, 63 (23), 15050-15071. https://doi.org/10.1021/acs.jmedchem.ocO1732.
(39) Dietrich, J. D.; Longenecker. K. L.; Wilson. N. S.; Goess, C.; Panchal, S. C.; Swann, S. L.; Petros, A. M.; Hobson, A. D.; Ihle, D.; Song, D.; Richardson. P.; Comess, K. M.; Cox, P. B.; Dombrowski, A.; Sarris, K.; Donnelly-Roberts, D. L.; Duignan, D. B.; Gomtsyan, A.; Jung, P.; Krueger, A. C.; Mathieu, S.; McClure, A.; Stoll, V. S.; Wetter, J.; Mankovich, J. A.; Hajduk, P. J.; Vasudevan, A.; Stoffel, R. H.; Sun, C. Development of Orally Efficacious Allosteric Inhibitors of TNFα via Fragment-Based Drug Design. J. Med. Chem. 2021, 64 (1), 417-429. https://doi.org/10.1021/acs.jmedchem.Oc01280.
(40) Nakao, S.; Ogata, Y.; Shimizu-Sasaki, E.; Yamazaki, M.; Furuyama, S.; Sugiya, H. Activation of NFκB Is Necessary for IL-1β-Induced Cyclooxygenase-2 (COX-2) Expression in Human Gingival Fibroblasts. Molecular and Cellular Biochemistry 2000, 209 (½), 113-118. https:/doi.org/10.1023/A:1007155525020.
(41) Ramadan, A. M.; Daguindau, E.; Rech, J. C.; Chinnaswa-mv. K.; Zhang, J.; Hura, G. L.; Griesenauer, B.; Bolten, Z.; Robida, A.; Larsen, M.; Stuckey, J. A.; Yang, C. Y.; Paczesny, S. From Proteomics to Discovery of First-in-Class ST2 Inhibitors Active in Vivo. JCI Insight 2018, 3 (14), e99208. https://doi.org/10.1172/jci.insight. 99208.
(42) Todorović, V.; Su, Z.; Putman, C. B.; Kakavas, S. J.; Salte, K. M.; McDonald, H. A.; Wetter, J. B.; Paulsboe, S. E.; Sun, Q.; Ger-stein, C. E.; Medina, L.; Sielaff, B.; Sadhukhan, R.; Stockmann, H.; Richardson, P. L.; Qiu. W.; Argiriadi, M. A.; Henry, R. F.; Herold, J. M.; Shotwell, J. B.; McGaraughty, S. P.; Honore, P.; Gopalakrishnan, S. M.; Sun, C. C.; Scott, V. E. Small Molecule IL-36γ Antagonist as a Novel Therapeutic Approach for Plaque Psoriasis. Sci Rep 2019, 9 (1), 9089. https://doi.org/10.1038/s41598-019-45626-w.
(43) Zyzynska-Granica, B.; Trzaskowski, B.; Niewieczerzal, S.; Filipek, S.; Zegrocka-Stendel, O.; Dutkiewicz, M.; Krzeczyntski, P.; Kowalewska, M.; Koziak, K. Pharmacophore Guided Discovery of Small-Molecule Interleukin 15 Inhibitors. European Journal of Medicinal Chemistry 2017, 136, 543-547. https://doi.org/10.1016/j.ejmech. 2017.05.034.
(44) Varghese, T. M.; Dudas, P. L.; Allen, S. J.; Schneeweis, J. E., Finley, M. F. A. Optimization of a High-Throughput Cell-Based Screening Strategy to Identify Small-Molecule Inhibitors of IL-23 Signaling. SLAS Discovery 2021, 26 (1), 122-129. https://doi.org/10.1177/2472555220923362.
(45) Braisted, A. C.; Oslob, J. D.; Delano, W. L.; Hyde, J.; McDowell, R. S.; Waal, N. Yu, C.; Arkin, M. R.; Raimundo, B. C. Dis-covery of a Potent Small Molecule IL-2 Inhibitor through Frag-ment Assembly. J. Am. Chem. Soc. 2003, 125 (13), 3714-3715. https://doi.org/10.1021/ja034247i.
(46) Thanos, C. D.; Randal, M.; Wells, J. A. Potent Small-Molecule Binding to a Dynamic Hot Spot on IL-2. J. Am. Chem. Soc. 2003, 125 (50), 15280-15281. https://doi.org/10.1021/ja0382617.
(47) Tilley, J. W.; Chen, L.; Fry, D. C.; Emerson, S. D.; Powers, G. D.; Biondi, D.; Varnell, T.; Trilles, R.; Guthrie, R.; Mennona, F.; Kaplan, G.; LeMahieu, R. A.; Carson, M.; Han, R. J.; Liu. C. M.; Pa-lermo, R.; Ju, G. Identification of a Small Molecule Inhibitor of the IL-2/IL-2Rα Receptor Interaction Which Binds to IL-2. J. Am. Chem. Soc. 1997, 119 (32), 7589-7590. https://doi.org/10.1021/ja970702x.
(48) Quinnell, S. P.; Leifer, B. S.; Nestor. S. T.; Tan, K.; Sheehy, D. F.; Ceo, L.; Doyle, S. K.; Koehler, A. N.; Vegas, A. J. A Small-Molecule Inhibitor to the Cytokine Interleukin-4. ACS Chem. Biol. 2020, 15 (10), 2649-2654. https://doi.org/10.1021/acschembio. 0c00615.
(49) Serry, A. M.; Luik, S.; Laufer, S.; Abadi, A. H. One-Pot Syn-thesis of 4,6-Diaryl-2-Oxo(Imino)-1,2-Dihydropyridine-3-Carbonitrile; a New Scaffold for P38a MAP Kinase Inhibition. J. Comb. Chem. 2010, 12 (4), 559-565. https://doi.org/10.1021/cc1000488.
(50) Kim. J. E.; Jung, K.; Kim, J. A.; Kim, S. H.; Park, H. S.; Kim, Y. S. Engineering of Anti-Human Interleukin-4 Receptor Alpha Antibodies with Potent Antagonistic Activity. Sci Rep 2019, 9 (1), 7772. https://doi.org/10.1038/s41598-019-44253-9.
(51) Obmolova, G.; Teplyakov, A.; Malia, T. J.; Keough. E.; Luo, J.; Sweet, R.; Jacobs, S. A.; Yi, F.; Hippensteel, R.; O'Neil, K. T.; Gilli-land, G. L. Induced Conformational Change in Human IL-4 upon Binding of a Signal-neutralizing DARP In. Proteins 2015, 83 (6), 1191-1197. https://doi.org/10.1002/prot. 24815.
(52) Hou. J.; Schindler, U.; Henzel, W. J.; Ho, T. C.; Brasseur, M.; McKnight, S. L. An Interleukin-4-Induced Transcription Factor: IL-4 Stat. Science 1994, 265 (5179), 1701-1706. https://doi.org/10.1126/science. 8085155.
(53) Heller, N. M.; Qi, X.; Junttila, I. S.; Shirey, K. A.; Vogel, S. N.; Paul, W. E.; Keegan, A. D. Type I IL-4Rs Selectively Activate IRS-2 to Induce Target Gene Expression in Macrophages. Sci. Signal. 2008, 1 (51). https://doi.org/10.1126/scisignal. 1164795.
(54) Wick, K. R.; Berton, M. T. IL-4 Induces Serine Phos-phorylation of the STAT6 Transactivation Domain in B Lympho-cytes. Molecular Immunology 2000, 37 (11), 641-652. https://doi.org/10.1016/S0161-5890(00)00088-2.
(55) Pesu, M.; Takaluoma, K.; Aittomaki, S.; Lagerstedt, A.; Saksela, K.; Kovanen, P. E.; Silvennoinen, O. Interleukin-4-Induced Transcriptional Activation by Stat6 Involves Multiple Ser-ine/Threonine Kinase Pathways and Serine Phosphorylation of Stat6. Blood 2000, 95 (2), 494-502. https://doi.org/10.1182/blood.V95.2.494.
(56) Leonard, W. J.; Lin, J. X.; O'Shea. J. J. The Fc Family of Cy-tokines: Basic Biology to Therapeutic Ramifications. Immunity 2019, 50 (4), 832-850. https://doi.org/10.1016/j.immuni. 2019.03.028.
(57) McCormick, S. M.; Heller, N. M. Commentary: IL-4 and IL-13 Receptors and Signaling. Cytokine 2015, 75 (1), 38-50. https://doi.org/10.1016/j.cyto. 2015.05.023.
(58) Hage, T.; Sebald, W.; Reinemer, P. Crystal Structure of the Interleukin-4/Receptor α Chain Complex Reveals a Mosaic Binding Interface. Cell 1999, 97 (2), 271-281. https://doi.org/10.1016/S0092-8674(00)80736-9.
(59) Scott, G.; Asrat, S.; Allinne. J.; Keat Lim, W.; Nagashima, K.; Birchard, D.; Srivatsan, S.; Ajithdoss, D. K.; Oyejide, A.; Ben, L. H.; Walls, J.; Le Floc'h, A.; Yancopoulos, G. D.; Murphy, A. J.; Sleeman, M. A.; Orengo, J. M. IL-4 and IL-13, Not Eosinophils, Drive Type 2 Airway Inflammation, Remodeling and Lung Function Decline. Cytokine 2023, 162, 156091. https://doi.org/10.1016/j.cyto. 2022.156091.
(60) Le Floc'h, A.; Allinne, J.; Nagashima, K.; Scott, G.; Birchard, D.; Asrat, S.; Bai, Y.; Lim, W. K.; Martin, J.; Huang, T.; Potocky, T. B.; Kim, J. H.; Rafique, A.; Papadopoulos, N. J., Stahl, N.; Yancopoulos. G. D.; Murphy, A. J.; Sleeman, M. A.; Orengo, J. M. Dual Blockade of IL-4 and IL-13 with Dupilumab, an IL-4Rα Anti-body, Is Required to Broadly Inhibit Type 2 Inflammation. Allergy 2020, 75 (5), 1188-1204. https://doi.org/10.1111/all. 14151.
(61) De Groot, A. E.; Myers, K. V.; Krueger, T. E. G.; Brennen, W. N.; Amend, S. R.; Pienta, K. J. Targeting Interleukin 4 Receptor Alpha on Tumor-Associated Macrophages Reduces the pro-Tumor Macrophage Phenotype. Neoplasia 2022, 32, 100830. https://doi.org/10.1016/j.neo. 2022.100830.

### EMBODIMENTS

### Embodiment 1

A compound of formula (I):

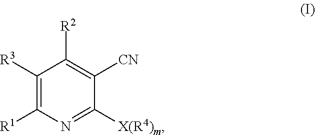

or a pharmaceutically acceptable salt, solvate, hydrate, polymorph, co-crystal, tautomer, stereoisomer, isotopically labeled compound, or prodrug thereof, wherein:
X is N, S, O, or halo;
each of R¹ and R² is independently optionally substituted alkyl, optionally substituted aryl, optionally substituted heteroaryl, optionally substituted heterocyclyl, optionally substituted amino, optionally substituted alkoxy, hydroxyl, or optionally substituted carbocyclyl, wherein at least one of R¹ and R² is optionally substituted cyclopropyl, optionally substituted cyclobutyl, or optionally substituted bicyclopentanyl;
R³ is hydrogen or optionally substituted alkyl,
each R⁴ is independently hydrogen, optionally substituted alkyl, a nitrogen protecting group when appended to a nitrogen, an oxygen protecting group when appended to an oxygen, a sulfur protecting group when appended to a sulfur, or, where X is N, two instances of R⁴, together with the nitrogen atom to which they are attached, form an optionally substituted heterocyclyl or heteroaryl; and
m is 0, 1, or 2.

### Embodiment 2

The compound of embodiment 1, or a pharmaceutically acceptable salt, solvate, hydrate, polymorph, co-crystal, tautomer, stereoisomer, isotopically labeled compound, or prodrug thereof, wherein the compound is of Formula (I-a), (I-b), or (1-c):

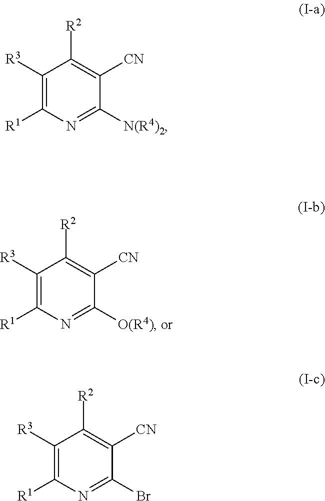

wherein each R⁴ is independently hydrogen, optionally substituted CI-alkyl, or two instances of R⁴, together with the atom to which they are attached, form an optionally substituted heterocyclyl or heteroaryl; and
R³ is hydrogen or optionally substituted C₁₋₆ alkyl.

### Embodiment 3

The compound of embodiment 1 or 2, or a pharmaceutically acceptable salt, solvate, hydrate, polymorph, co-crystal, tautomer, stereoisomer, isotopically labeled compound, or prodrug thereof, wherein one of R¹ and R² is optionally substituted aryl, optionally substituted heteroaryl, optionally substituted heterocyclyl, optionally substituted amino, optionally substituted alkoxy, hydroxyl, or optionally substituted carbocyclyl.

### Embodiment 4

The compound of embodiment 1 or 2, or a pharmaceutically acceptable salt, solvate, hydrate, polymorph, co-crystal, tautomer, stereoisomer, isotopically labeled compound, or prodrug thereof, wherein one of R¹ and R² is optionally substituted aryl, optionally substituted heteroaryl, optionally substituted heterocyclyl, or optionally substituted C₁₋₆ alkyl.

### Embodiment 5

The compound of any one of embodiments 1-4 or a pharmaceutically acceptable salt, solvate, hydrate, polymorph, co-crystal, tautomer, stereoisomer, isotopically labeled compound, or prodrug thereof, wherein one of R¹ and R² is optionally substituted aryl or optionally substituted heteroaryl.

### Embodiment 6

The compound of any one of embodiments 1-5, or a pharmaceutically acceptable salt, solvate, hydrate, polymorph, co-crystal, tautomer, stereoisomer, isotopically labeled compound, or prodrug thereof, wherein one of R¹ and R² is aryl or heteroaryl substituted with at least one of hydroxyl, alkoxyl, halogen, amino, alkyl, acyl, or heterocyclyl.

### Embodiment 7

The compound of any one of embodiments 1-6, or a pharmaceutically acceptable salt, solvate, hydrate, polymorph, co-crystal, tautomer, stereoisomer, isotopically labeled compound, or prodrug thereof, wherein one of R¹ and R² is optionally substituted phenyl, biphenyl, thiophenyl, naphthyl, furanyl, or pyridinyl.

### Embodiment 8

The compound of embodiments 1-6, or a pharmaceutically acceptable salt, solvate, hydrate, polymorph, co-crystal, tautomer, stereoisomer, isotopically labeled compound, or prodrug thereof, wherein one of R¹ and R² is aryl substituted with at least one of hydroxyl, alkoxyl, halogen, amino, alkyl, acyl, or heteroaryl.

### Embodiment 9

The compound of embodiment 8, or a pharmaceutically acceptable salt, solvate, hydrate, polymorph, co-crystal, tautomer, stereoisomer, isotopically labeled compound, or prodrug thereof, wherein one of R¹ and R² is phenyl substituted with hydroxyl, halogen, or alkyl.

### Embodiment 10

The compound of any one of embodiments 1-6, or a pharmaceutically acceptable salt, solvate, hydrate, polymorph, co-crystal, tautomer, stereoisomer, isotopically labeled compound, or prodrug thereof, wherein one of R¹ and R² is optionally substituted heteroaryl.

### Embodiment 11

The compound of embodiment 10, or a pharmaceutically acceptable salt, solvate, hydrate, polymorph, co-crystal, tautomer, stereoisomer, isotopically labeled compound, or prodrug thereof, wherein one of R¹ and R² is unsubstituted heteroaryl.

### Embodiment 12

The compound of embodiment 1, 2, or 4, or a pharmaceutically acceptable salt, solvate. hydrate, polymorph, co-crystal, tautomer, stereoisomer, isotopically labeled compound, or prodrug thereof, wherein one of R¹ and R² is optionally substituted C₁₋₆ alkyl.

### Embodiment 13

The compound of any one of embodiments 1-12 or a pharmaceutically acceptable salt, solvate, hydrate, polymorph, co-crystal, tautomer, stereoisomer, isotopically labeled compound, or prodrug thereof, wherein at least one of R¹ and R² is cyclopropyl, cyclobutyl, or bicyclopentanyl.

### Embodiment 14

The compound of embodiment 1 or 2, or a pharmaceutically acceptable salt, solvate, hydrate. polymorph, co-crystal, tautomer, stereoisomer, isotopically labeled compound, or prodrug thereof, wherein at least one of R¹ and R² is selected from the group consisting of:

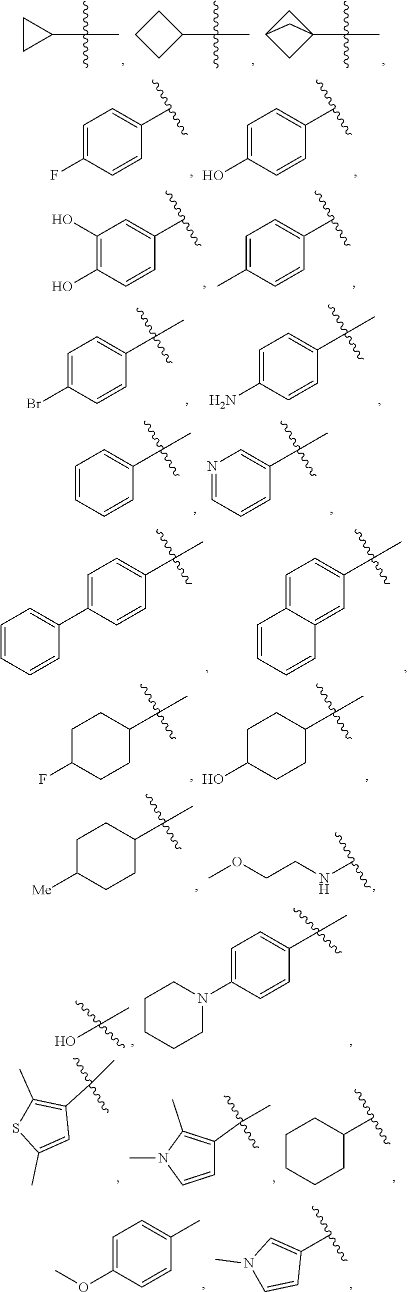

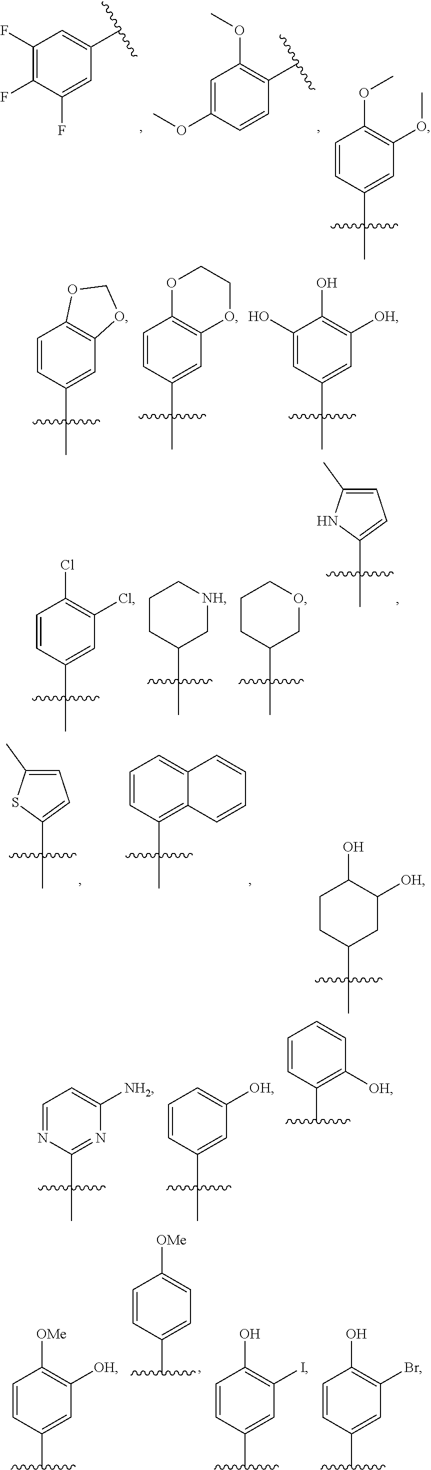

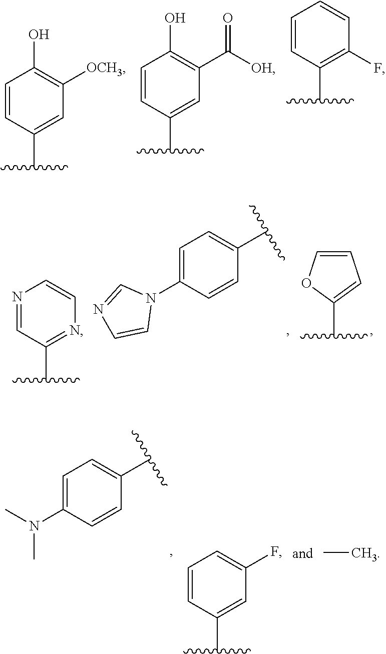

### Embodiment 15

The compound of embodiment 14, or a pharmaceutically acceptable salt, solvate, hydrate, polymorph, co-crystal, tautomer, stereoisomer, isotopically labeled compound, or prodrug thereof, wherein each of R¹ and R² is independently selected from the group consisting of:

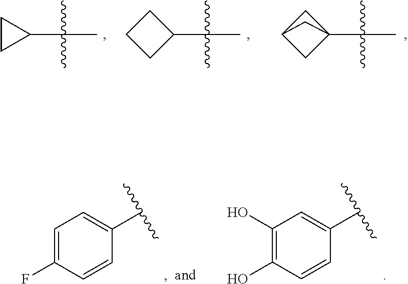

### Embodiment 16

The compound of embodiment 15. or a pharmaceutically acceptable salt, solvate, hydrate, polymorph, co-crystal, tautomer, stereoisomer, isotopically labeled compound, or prodrug thereof, wherein R¹ is

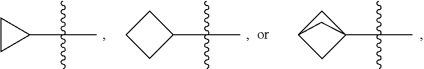

### and R² is

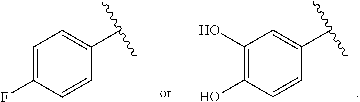 [SMILES: *c1ccc(F)cc1 ; *c1ccc(O)c(O)c1]

### Embodiment 17

The compound of embodiment 16, or a pharmaceutically acceptable salt, solvate, hydrate. polymorph, co-crystal, tautomer, stereoisomer, isotopically labeled compound, or prodrug thereof, wherein R¹ is

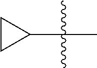 [SMILES: *C1CC1]

### and R² is

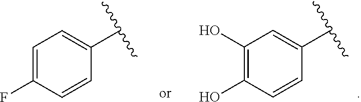 [SMILES: *c1ccc(F)cc1 ; *c1ccc(O)c(O)c1]

### Embodiment 18

The compound of embodiment 16, or a pharmaceutically acceptable salt, solvate. hydrate, polymorph, co-crystal, tautomer, stereoisomer, isotopically labeled compound, or prodrug thereof, wherein R¹ is

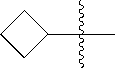 [SMILES: *C1CCC1]

### and R² is

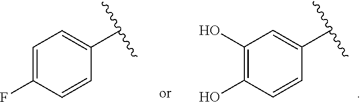 [SMILES: *c1ccc(F)cc1 ; *c1ccc(O)c(O)c1]

### Embodiment 19

The compound of embodiment 16, or a pharmaceutically acceptable salt, solvate, hydrate, polymorph, co-crystal, tautomer, stereoisomer, isotopically labeled compound, or prodrug thereof, wherein R¹ is

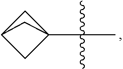 [SMILES: *C12CC(C1)C2]

### and R² is

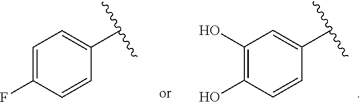 [SMILES: *c1ccc(F)cc1 ; *c1ccc(O)c(O)c1]

### Embodiment 20

The compound of embodiment any one of embodiments 1-19, or a pharmaceutically acceptable salt, solvate, hydrate, polymorph, co-crystal, tautomer, stereoisomer, isotopically labeled compound, or prodrug thereof, wherein R² is

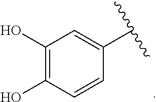 [SMILES: *c1ccc(O)c(O)c1]

### Embodiment 21

The compound of embodiment 15, or a pharmaceutically acceptable salt, solvate, hydrate, polymorph, co-crystal, tautomer, stereoisomer, isotopically labeled compound, or prodrug thereof, wherein R² is

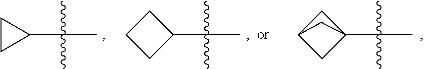

### and R¹ is

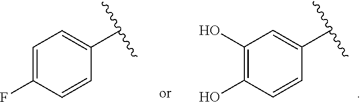 [SMILES: *c1ccc(F)cc1 ; *c1ccc(O)c(O)c1]

### Embodiment 22

The compound of embodiment 21, or a pharmaceutically acceptable salt, solvate, hydrate, polymorph, co-crystal, tautomer, stereoisomer, isotopically labeled compound, or prodrug thereof, wherein R² is

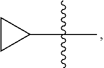 [SMILES: *C1CC1]

### and R¹ is

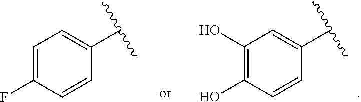 [SMILES: *c1ccc(F)cc1 ; *c1ccc(O)c(O)c1]

### Embodiment 23

The compound of embodiment 21, or a pharmaceutically acceptable salt, solvate, hydrate, polymorph, co-crystal, tautomer, stereoisomer, isotopically labeled compound, or prodrug thereof, wherein R² is

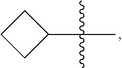 [SMILES: *C1CCC1]

### and R¹ is

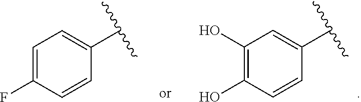 [SMILES: *c1ccc(F)cc1 ; *c1ccc(O)c(O)c1]

### Embodiment 24

The compound of embodiment 21, or a pharmaceutically acceptable salt, solvate, hydrate, polymorph, co-crystal, tautomer, stereoisomer, isotopically labeled compound, or prodrug thereof, wherein R² is

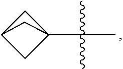 [SMILES: *C12CC(C1)C2]

### and R¹ is

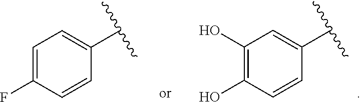 [SMILES: *c1ccc(F)cc1 ; *c1ccc(O)c(O)c1]

### Embodiment 25

The compound of any one of embodiments 1-24, or a pharmaceutically acceptable salt, solvate, hydrate, polymorph, co-crystal, tautomer, stereoisomer, isotopically labeled compound, or prodrug thereof, wherein R¹ is

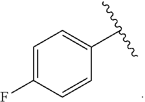 [SMILES: *c1ccc(F)cc1]

### Embodiment 26

The compound of embodiment 1, wherein the compound is of the formula:

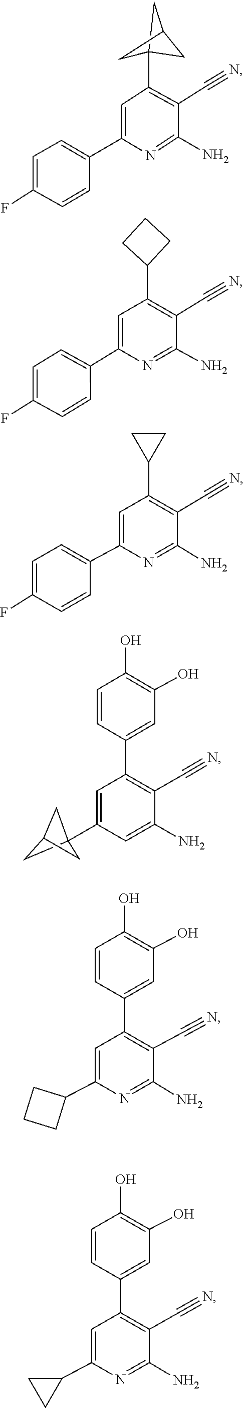 [SMILES: N#Cc1c(C23CC(C2)C3)cc(-c2ccc(F)cc2)nc1N ; N#Cc1c(C2CCC2)cc(-c2ccc(F)cc2)nc1N ; N#Cc1c(C2CC2)cc(-c2ccc(F)cc2)nc1N ; N#Cc1c(N)cc(C23CC(C2)C3)cc1-c1ccc(O)c(O)c1 ; N#Cc1c(-c2ccc(O)c(O)c2)cc(C2CCC2)nc1N ; N#Cc1c(-c2ccc(O)c(O)c2)cc(C2CC2)nc1N]

or a pharmaceutically acceptable salt, solvate, hydrate, polymorph, co-crystal, tautomer, stereoisomer, isotopically labeled compound, or prodrug thereof.

### Embodiment 27

A pharmaceutical composition comprising a compound of embodiments 1-26, or a pharmaceutically acceptable salt, solvate, hydrate, polymorph, co-crystal, tautomer, stereoisomer, isotopically labeled compound, or prodrug thereof, and a pharmaceutically acceptable carrier.

### Embodiment 28

The pharmaceutical composition of embodiment 27, further comprising an additional pharmaceutical agent.

### Embodiment 29

The pharmaceutical composition of embodiment 28, wherein the additional pharmaceutical agent is selected from the group consisting of [LIST].

### Embodiment 30

A therapeutic composition comprising the compound of any one of embodiments 1-26 or the pharmaceutical composition of any one of embodiments 27-0, and an at least one second therapeutic molecule.

### Embodiment 31

The therapeutic composition of embodiment 29, wherein the compound and the at least one second therapeutic molecule are conjugated or ligated to one another.

### Embodiment 32

The therapeutic composition of embodiment 30, wherein the compound and the at least one second therapeutic molecule are both present in or on a scaffold material or molecule.

### Embodiment 33

Use of the compound of any one of embodiments 1-26, or a pharmaceutically acceptable salt, solvate, hydrate, polymorph, co-crystal, tautomer, stereoisomer, isotopically labeled compound, or prodrug thereof, or the pharmaceutical composition of any one of embodiments 27-0, or the therapeutic composition of any one of embodiments 29-31, in the manufacture of a medicament for treating a disease.

### Embodiment 34

A method of treating a disease in a subject in need thereof, the method comprising administering to the subject in need thereof a therapeutically effective amount of a compound of any one of embodiments 1-26, or a pharmaceutically acceptable salt, solvate, hydrate, polymorph, co-crystal, tautomer, stereoisomer, isotopically labeled derivative, or prodrug thereof, or the pharmaceutical composition of any one of embodiments 27-0, or the therapeutic composition of any one of embodiments 29-31.

### Embodiment 35

The method of embodiment 33, wherein the disease is a proliferative disease, immune disease, autoimmune disorder, inflammatory disorder, allergies, infection, fibrosis or [LIST].

### Embodiment 36

The method of embodiment 34, wherein the disease is selected from the group consisting of asthma, rheumatoid arthritis, allergies, cancer, atopic dermatitis, eosinophilic esophagitis, prurigo nodularis, chronic rhinosinusitis with nasal polyposis, chronic obstructive pulmonary disease (COPD), tissue/organ fibrosis, implant fibrosis, systemic lupus erythematosus, bullous pemphigoid, chronic spontaneous urticaria, Graves' disease, multiple sclerosis, pemphigus, uveitis [LIST].

### Embodiment 37

The method of any one of embodiments 33-35, wherein the disease is a disease associated with overexpression and/or aberrant activity of IL-4

### Embodiment 38

The method of any one of embodiments 33-36, wherein the disease is a proliferative disease.

### Embodiment 39

The method of embodiment 37, wherein the proliferative disease is cancer.

### Embodiment 40

The method of embodiment 38, wherein the cancer is carcinoma, sarcoma, lymphoma, germinoma[LIST].

### Embodiment 41

The method of embodiment 33-36, wherein the disease is an immune disorder.

### Embodiment 42

The method of embodiment 41, wherein the immune disorder is asthma, rheumatoid arthritis, allergies, atopic dermatitis, eosinophilic esophagitis, prurigo nodularis, chronic rhinosinusitis with nasal polyposis, chronic obstructive pulmonary disease (COPD), tissue/organ fibrosis, implant fibrosis, systemic lupus erythematosus, bullous pemphigoid, chronic spontaneous urticaria, Graves' disease, multiple sclerosis, pemphigus, uveitis [LIST].

### Embodiment 43

The method of embodiment 33-36, wherein the disease is an inflammatory disorder.

### Embodiment 44

The method of embodiment 43, wherein the inflammatory disorder is asthma, allergies, atopic dermatitis, eosinophilic esophagitis, prurigo nodularis, chronic rhinosinusitis with nasal polyposis, chronic obstructive pulmonary disease (COPD), tissue/organ fibrosis, implant fibrosis, uveitis [LIST].

### Embodiment 45

The method of embodiment 33-36, wherein the disease is an autoimmune disease.

### Embodiment 46

The method of embodiment 45, wherein the autoimmune disease is rheumatoid arthritis, systemic lupus erythematosus, bullous pemphigoid, chronic spontaneous urticaria, Graves' disease, multiple sclerosis, pemphigus [LIST].

### Embodiment 47

A method of reducing an inflammatory response in a subject in need thereof, the method comprising administering to the subject a composition of any one of embodiments 1-26 or a pharmaceutically acceptable salt, solvate, hydrate, polymorph, co-crystal, tautomer, stereoisomer, isotopically labeled compound, or prodrug thereof, or the pharmaceutical composition of any one of embodiments 27-0, or the therapeutic composition of any one of embodiments 29-31.

### Embodiment 48

A method of inhibiting the activity of a cytokine in a subject in need thereof, the method comprising administering to the subject an effective amount of a compound of any one of 1-26 or a pharmaceutically acceptable salt, solvate, hydrate, polymorph, co-crystal, tautomer, stereoisomer, isotopically labeled compound, or prodrug thereof, or the pharmaceutical composition of any one of embodiments 27-0, or the therapeutic composition of any one of embodiments 29-31.

### Embodiment 49

The method of embodiment 48, wherein the cytokine is IL-4.

### Embodiment 50

The method of embodiment 48 or 49, wherein inhibition is selective for IL-4 over IL-13.

### Embodiment 51

The method of any one of embodiments 48-50, wherein the method further comprises inhibition of IL-4 induced STAT-6 phosphorylation.

### Embodiment 52

The method of any one of embodiments 33-51, further comprising administering to the subject in need thereof an additional therapy.

### Embodiment 53

The method of embodiment 52, wherein the additional therapy is a cytotoxic chemotherapy, epigenetic modifier, glucocorticoid, immunotherapy, cell-based therapies, nucleic acid therapies, vaccines, radiotherapy, protein degraders, antibody-drug conjugates, gene therapy or [LIST].

### Embodiment 54

The method of any one of embodiments 33-53, wherein the subject is a human.

### Embodiment 55

The method of any one of embodiments 33-53, wherein the subject is a non-human mammal.

### Embodiment 56

A method of inhibiting the activity of IL-4 in a biological sample or cell, the method comprising administering to the biological sample or cell an effective amount of a compound of any one of embodiments 1-26 or a pharmaceutically acceptable salt, solvate, hydrate, polymorph, co-crystal, tautomer, stereoisomer, isotopically labeled compound, or prodrug thereof, or the pharmaceutical composition of any one of embodiments 27-0, or the therapeutic composition of any one of embodiments 29-31.

### Embodiment 57

The method of embodiment 56, wherein the biological sample or cell is in vivo.

### Embodiment 58

The method of embodiment 56, wherein the biological sample or cell is ex vivo.

### Embodiment 59

The method of any one of embodiments 56-58, wherein the cell is a malignant cell or premalignant cell.

### Embodiment 60

The method of any one of embodiments 36-59, wherein IL-4 is a wild-type cytokine or mutant cytokine.

### Embodiment 61

A kit comprising:
a compound of any one of embodiments 1-26 or a pharmaceutically acceptable salt, solvate, hydrate, polymorph, co-crystal, tautomer, stereoisomer, isotopically labeled compound, or prodrug thereof, or the pharmaceutical composition of any one of embodiments 27-0, or the therapeutic composition of any one of embodiments 29-31; and
instructions for using the compound, or a pharmaceutically acceptable salt, solvate, hydrate, polymorph, co-crystal, tautomer, stereoisomer, isotopically labeled compound, or prodrug thereof, or the pharmaceutical composition or the therapeutic composition.

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1-61. (canceled)

62. A compound of formula (I): 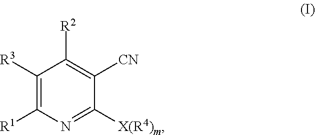
or a pharmaceutically acceptable salt, solvate, hydrate, polymorph, co-crystal, tautomer, stereoisomer, isotopically labeled compound, or prodrug thereof, wherein:
X is N, S, O, or halo;
each of R¹ and R² is independently optionally substituted alkyl, optionally substituted aryl, optionally substituted heteroaryl, optionally substituted heterocyclyl, optionally substituted amino, optionally substituted alkoxy, hydroxyl, or optionally substituted carbocyclyl, wherein at least one of R¹ and R² is optionally substituted cyclopropyl, optionally substituted cyclobutyl, or optionally substituted bicyclopentanyl;
R³ is hydrogen or optionally substituted alkyl,
each R⁴ is independently hydrogen, optionally substituted alkyl, a nitrogen protecting group when appended to a nitrogen, an oxygen protecting group when appended to an oxygen, a sulfur protecting group when appended to a sulfur, or, where X is N, two instances of R⁴, together with the nitrogen atom to which they are attached, form an optionally substituted heterocyclyl or heteroaryl; and
m is 0, 1, or 2.

63. The compound of claim 62, or a pharmaceutically acceptable salt, solvate, hydrate, polymorph, co-crystal, tautomer, stereoisomer, isotopically labeled compound, or prodrug thereof, wherein the compound is of Formula (I-a), (I-b), (I-c), or (I-d): 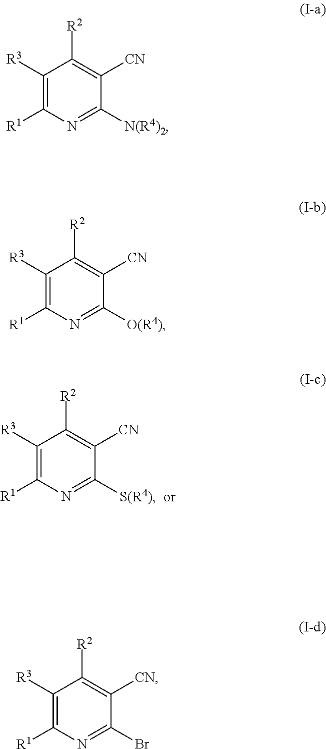
wherein each R⁴ is independently hydrogen, optionally substituted C₁₋₆ alkyl, or two instances of R⁴, together with the atom to which they are attached, form an optionally substituted heterocyclyl or heteroaryl; and
R³ is hydrogen or optionally substituted C₁₋₆ alkyl.

64. The compound of claim 63, or a pharmaceutically acceptable salt, solvate, hydrate, polymorph, co-crystal, tautomer, stereoisomer, isotopically labeled compound, or prodrug thereof, wherein the compound is of Formula (I-a)

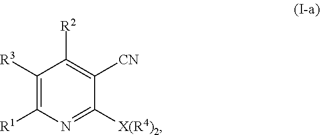

65. The compound of claim 62, or a pharmaceutically acceptable salt, solvate, hydrate, polymorph, co-crystal, tautomer, stereoisomer, isotopically labeled compound, or prodrug thereof, wherein one of R¹ and R² is optionally substituted aryl, optionally substituted heteroaryl, optionally substituted heterocyclyl, or optionally substituted C₁₋₆ alkyl.

66. The compound of claim 65, or a pharmaceutically acceptable salt, solvate, hydrate, polymorph, co-crystal, tautomer, stereoisomer, isotopically labeled compound, or prodrug thereof, wherein one of R¹ and R² is aryl or heteroaryl substituted with at least one of hydroxyl, alkoxyl, halogen, amino, alkyl, acyl, or heterocyclyl.

67. The compound of claim 66, or a pharmaceutically acceptable salt, solvate, hydrate, polymorph, co-crystal, tautomer, stereoisomer, isotopically labeled compound, or prodrug thereof, wherein one of R¹ and R² is optionally substituted phenyl, biphenyl, thiophenyl, naphthyl, furanyl, or pyridinyl.

68. The compound of claim 62, or a pharmaceutically acceptable salt, solvate, hydrate, polymorph, co-crystal, tautomer, stereoisomer, isotopically labeled compound, or prodrug thereof, wherein at least one of R¹ and R² is selected from the group consisting of:

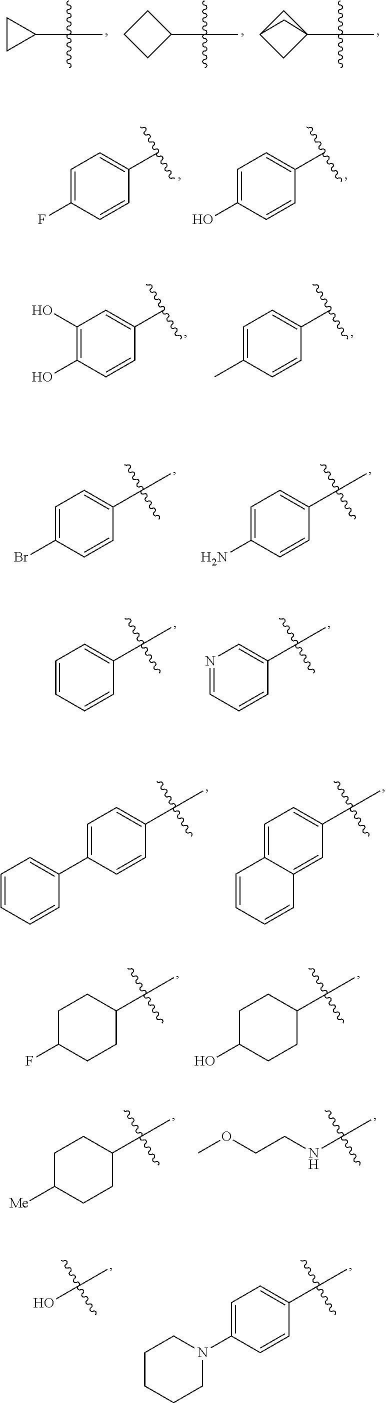

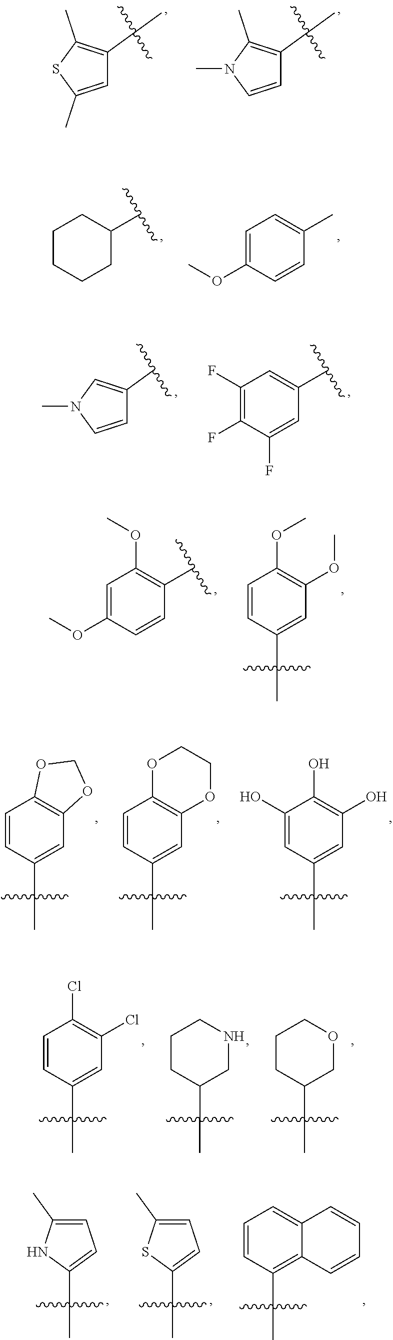

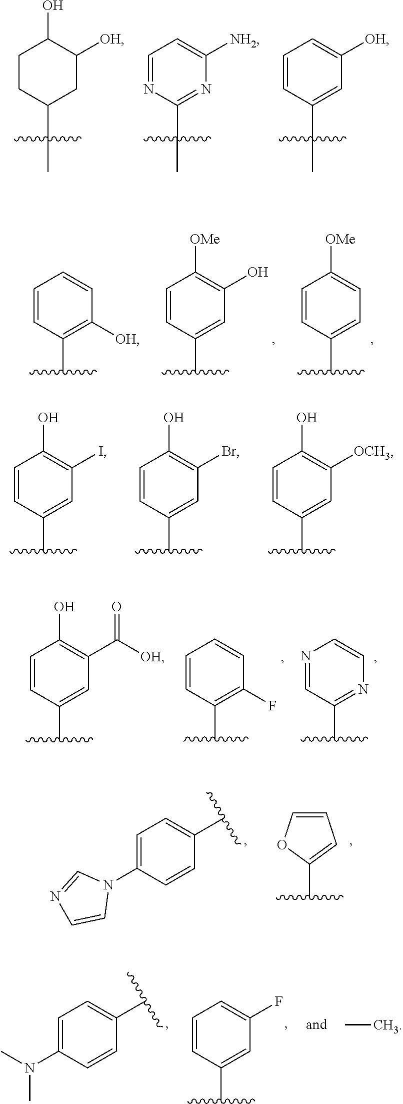

69. The compound of claim 62, or a pharmaceutically acceptable salt, solvate, hydrate, polymorph, co-crystal, tautomer, stereoisomer, isotopically labeled compound, or prodrug thereof, wherein each of R¹ and R² is independently selected from the group consisting of:

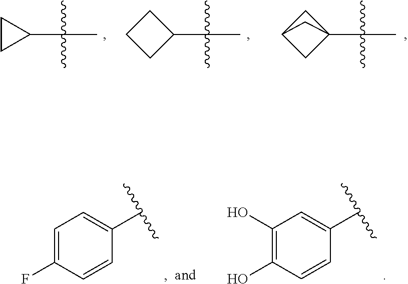

70. The compound of claim 69, or a pharmaceutically acceptable salt, solvate, hydrate, polymorph, co-crystal, tautomer, stereoisomer, isotopically labeled compound, or prodrug thereof, wherein R¹ is 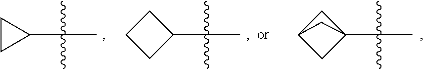
and R² is

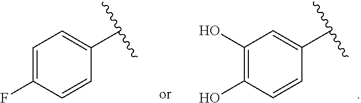 [SMILES: *c1ccc(F)cc1 ; *c1ccc(O)c(O)c1]

71. The compound of claim 70, or a pharmaceutically acceptable salt, solvate, hydrate, polymorph, co-crystal, tautomer, stereoisomer, isotopically labeled compound, or prodrug thereof, wherein R¹ is 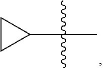 [SMILES: *C1CC1]
and R² is

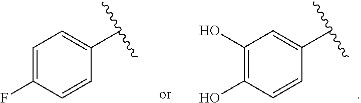 [SMILES: *c1ccc(F)cc1 ; *c1ccc(O)c(O)c1]

72. The compound of claim 70, or a pharmaceutically acceptable salt, solvate, hydrate, polymorph, co-crystal, tautomer, stereoisomer, isotopically labeled compound, or prodrug thereof, wherein R¹ is 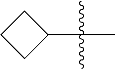 [SMILES: *C1CCC1]
and R² is

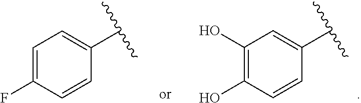 [SMILES: *c1ccc(F)cc1 ; *c1ccc(O)c(O)c1]

73. The compound of claim 70, or a pharmaceutically acceptable salt, solvate, hydrate, polymorph, co-crystal, tautomer, stereoisomer, isotopically labeled compound, or prodrug thereof, wherein R¹ is 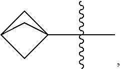 [SMILES: *C12CC(C1)C2]
and R² is

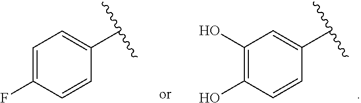 [SMILES: *c1ccc(F)cc1 ; *c1ccc(O)c(O)c1]

74. The compound of claim 69, or a pharmaceutically acceptable salt, solvate, hydrate, polymorph, co-crystal, tautomer, stereoisomer, isotopically labeled compound, or prodrug thereof, wherein R² is 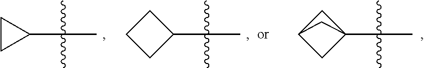
and R¹ is

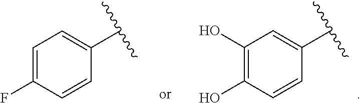 [SMILES: *c1ccc(F)cc1 ; *c1ccc(O)c(O)c1]

75. The compound of claim 74, or a pharmaceutically acceptable salt, solvate, hydrate, polymorph, co-crystal, tautomer, stereoisomer, isotopically labeled compound, or prodrug thereof, wherein R² is 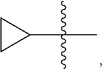 [SMILES: *C1CC1]
and R¹ is

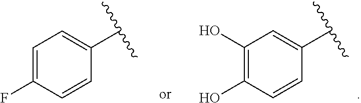 [SMILES: *c1ccc(F)cc1 ; *c1ccc(O)c(O)c1]

76. The compound of claim 74, or a pharmaceutically acceptable salt, solvate, hydrate, polymorph, co-crystal, tautomer, stereoisomer, isotopically labeled compound, or prodrug thereof, wherein R² is 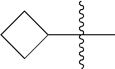 [SMILES: *C1CCC1]
and R¹ is

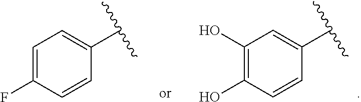 [SMILES: *c1ccc(F)cc1 ; *c1ccc(O)c(O)c1]

77. The compound of claim 74, or a pharmaceutically acceptable salt, solvate, hydrate, polymorph, co-crystal, tautomer, stereoisomer, isotopically labeled compound, or prodrug thereof, wherein R² is 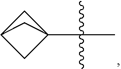 [SMILES: *C12CC(C1)C2]
and R¹ is

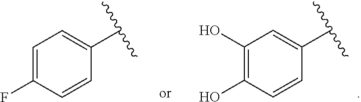 [SMILES: *c1ccc(F)cc1 ; *c1ccc(O)c(O)c1]

78. The compound of claim 62, wherein the compound is of the formula: 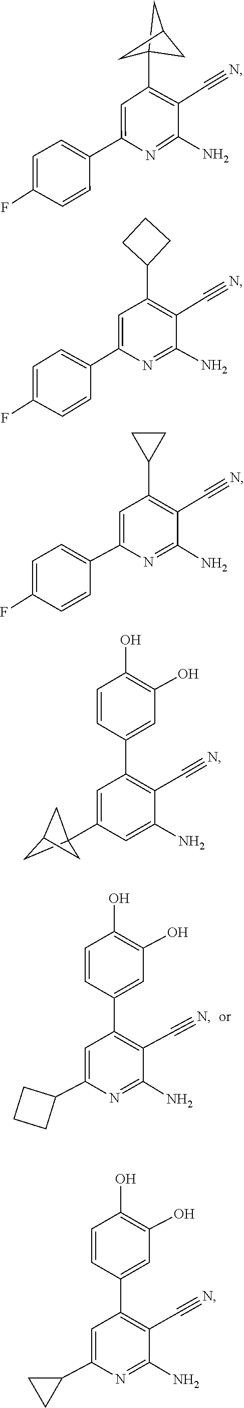 [SMILES: N#Cc1c(C23CC(C2)C3)cc(-c2ccc(F)cc2)nc1N ; N#Cc1c(C2CCC2)cc(-c2ccc(F)cc2)nc1N ; N#Cc1c(C2CC2)cc(-c2ccc(F)cc2)nc1N ; N#Cc1c(N)cc(C23CC(C2)C3)cc1-c1ccc(O)c(O)c1 ; N#Cc1c(-c2ccc(O)c(O)c2)cc(C2CCC2)nc1N ; N#Cc1c(-c2ccc(O)c(O)c2)cc(C2CC2)nc1N]
or a pharmaceutically acceptable salt, solvate, hydrate, polymorph, co-crystal, tautomer, stereoisomer, isotopically labeled compound, or prodrug thereof.

79. A pharmaceutical composition comprising a compound of claim 62, or a pharmaceutically acceptable salt, solvate, hydrate, polymorph, co-crystal, tautomer, stereoisomer, isotopically labeled compound, or prodrug thereof, and a pharmaceutically acceptable carrier.

80. The pharmaceutical composition of claim 79, further comprising an additional pharmaceutical agent.

81. A method of treating a disease in a subject in need thereof, the method comprising administering to the subject in need thereof a therapeutically effective amount of a compound of claim 62, or a pharmaceutically acceptable salt, solvate, hydrate, polymorph, co-crystal, tautomer, stereoisomer, isotopically labeled derivative, or prodrug thereof.

82. The method of claim 81, wherein the disease is a proliferative disease, immune disease, autoimmune disorder, inflammatory disorder, allergies, infection, or fibrosis.

83. The method of claim 81, wherein the disease is a disease associated with overexpression and/or aberrant activity of IL-4.

84. The method of claim 82, wherein the proliferative disease is cancer.

85. The method of claim 84, wherein the cancer is carcinoma, sarcoma, lymphoma, or germinoma.

86. The method of claim 84, wherein the cancer is not leukemia.

87. The method of claim 82, wherein the disease is an inflammatory disorder or autoimmune disorder.

88. A method of reducing an inflammatory response in a subject in need thereof, the method comprising administering to the subject a compound of claim 62, or a pharmaceutically acceptable salt, solvate, hydrate, polymorph, co-crystal, tautomer, stereoisomer, isotopically labeled compound, or prodrug thereof.

89. A method of inhibiting the activity of a cytokine in a subject in need thereof, the method comprising administering to the subject an effective amount of a compound of claim 62, or a pharmaceutically acceptable salt, solvate, hydrate, polymorph, co-crystal, tautomer, stereoisomer, isotopically labeled compound, or prodrug thereof.

90. A method of inhibiting the activity of IL-4 in a biological sample or cell, the method comprising administering to the biological sample or cell an effective amount of a compound of claim 62, or a pharmaceutically acceptable salt, solvate, hydrate, polymorph, co-crystal, tautomer, stereoisomer, isotopically labeled compound, or prodrug thereof.

91. A kit comprising:
a compound of claim 62 or a pharmaceutically acceptable salt, solvate, hydrate, polymorph, co-crystal, tautomer, stereoisomer, isotopically labeled compound, or prodrug thereof; and
instructions for using the compound, or a pharmaceutically acceptable salt, solvate, hydrate, polymorph, co-crystal, tautomer, stereoisomer, isotopically labeled compound, or prodrug thereof.
